(19) United States
(12) Patent Application Publication
(10) Pub. No.: US 2011/0053923 A1
(43) Pub. Date: Mar. 3, 2011

(54) CHEMICAL COMPOUNDS 610

(75) Inventors: Kevin Michael Foote, Macclesfield (GB); Johannes Wilhelmus Maria Nissink, Macclesfield (GB)

(73) Assignee: AstraZeneca, Sodertalje (SE)

(21) Appl. No.: 12/642,952

(22) Filed: Dec. 21, 2009

Related U.S. Application Data
(60) Provisional application No. 61/139,681, filed on Dec. 22, 2008.

(51) Int. Cl. A61K 31/537; C07D 413/14; A61K 31/5377; C07D 498/08; A61P 35/00

(52) U.S. Cl. 514/230.5; 544/122; 514/235.8; 544/105

(57) ABSTRACT

There is provided pyrimidinyl indole compounds of Formula (I),

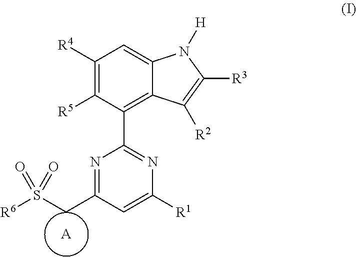

or pharmaceutically acceptable salts thereof, processes for their preparation, pharmaceutical compositions containing them and their use in therapy.

This application claims the benefit under 35 U.S.C. §119(e) of Application No U.S. 61/139,681 filed on 22 Dec. 2008.

The present invention relates to pyrimidinyl indole compounds, processes for their preparation, pharmaceutical compositions containing them and their use in therapy, for example in the treatment of proliferative disease such as cancer and particularly in disease mediated by Ataxia-telangiectasia mutated and RAD-3 related protein kinase inhibitors, commonly referred to as ATR.

ATR (also known as FRAP-Related Protein 1; FRP1; MEC1; SCKL; SECKL1) protein kinase is a member of the PI3-Kinase like kinase (PIKK) family of proteins that are involved in repair and maintenance of the genome and its stability (reviewed in Cimprich K. A. and Cortez D. 2008, Nature Rev. Mol. Cell Biol. 9:616-627). These proteins co-ordinate responses to DNA damage, stress and cell-cycle perturbation. Indeed ATM and ATR, two members of the family of proteins, share a number of downstream substrates that are themselves recognised components of the cell cycle and DNA-repair machinery e.g. Chk1, BRCA1, p53 (Lakin N D et al, 1999, Oncogene; Tibbets R S et al, 2000, Genes & Dev.). Whilst the substrates of ATM and ATR are to an extent shared, the trigger to activate the signalling cascade is not shared and ATR primarily responds to stalled replication forks (Nyberg K. A. et al., 2002, Ann. Rev. Genet. 36:617-656; Shechter D. et al. 2004, DNA Repair 3:901-908) and bulky DNA damage lesions such as those formed by ultraviolet (UV) radiation (Wright J. A. et al, 1998, Proc. Natl. Acad. Sci. USA, 23:7445-7450) or the UV mimetic agent, 4-nitroquinoline-1-oxide, 4NQO (Ikenaga M. et al. 1975, Basic Life Sci. 5b, 763-771).

Mutations of the ATR gene are rare and viability may only result under heterozygous or hypomorphic conditions. The only clear link between ATR gene mutations and disease exists in a few patients with Seckel syndrome which is characterized by growth retardation and microcephaly (O'Driscoll M et al, 2003 Nature Genet. Vol 3, 497-501). Disruptions of the ATR pathway leads to genomic instability, while ATR is activated by most cancer chemotherapies (Wilsker D et al, 2007, Mol. Cancer Ther. 6(4) 1406-1413). Moreover, duplication of the ATR gene has been described as a risk factor in rhabdomyosarcomas (Smith L et al, 1998, Nature Genetics 19, 39-46).

ATR is essential to the viability of replicating cells and is activated during S-phase to regulate firing of replication origins and to repair damaged replication forks (Shechter D et al, 2004, Nature cell Biology Vol 6 (7) 648-655). Damage to replication forks may arise due to exposure of cells to clinically relevant cytotoxic agents such as hydroxyurea (HU) and platinums (O'Connell and Cimprich 2005; 118, 1-6).

Conversely, sensitisation to chemotherapeutic agents can be achieved by modulation of ATR activity. It has thus been proposed that inhibition of ATR may prove to be an efficacious approach to future cancer therapy (Collins I. and Garret M. D., 2005, Curr. Opin. Pharmacol., 5:366-373; Kaelin W. G. 2005, Nature Rev. Cancer, 5:689-698). There is currently no clinical precedent for agents targeting ATR, although agents targeting the downstream signalling axis i.e. Chk1 are currently undergoing clinical evaluation (reviewed in Janetka J. W. et al. Curr Opin Drug Discov Devel, 2007, 10:473-486). However, DNA damage induced by exposure of tumour cells to cytotoxic chemotherapeutic agents such as hydroxyurea and platinum products gives rise to damaged replication forks, a trigger for ATR activation and its signalling to a number of cell-critical processes.

Biological assessment of the ability of ATR inhibitors to sensitise to a wide range of chemotherapies have been evaluated. Sensitisation of tumour cells to chemotherapeutic agents in cell growth assays has been noted and used to assess how well weak ATR inhibitors (such as Caffeine) will sensitise tumour cell lines to cytotoxic agents. (Wilsker D. et al, 2007, Mol Cancer Ther. 6 (4)1406-1413; Sarkaria J. N. et al, 1999, Cancer Res. 59, 4375-4382). Moreover, a reduction of ATR activity by siRNA or ATR knock-in using a dominant negative form of ATR in cancer cells has resulted in the sensitisation of tumour cells to the effects of a number of therapeutic or experimental agents such as antimetabolites (5-FU, Gemcitabine, Hydroxyurea, Metotrexate, Tomudex), alkylating agents (Cisplatin, Mitomycin C, Cyclophosphamide, MMS) or double-strand break inducers (Doxorubicin, Ionizing radiation) (Cortez D. et al. 2001, Science, 294:1713-1716; Collis S. J. et al, 2003, Cancer Res. 63:1550-1554; Cliby W. A. et al, 1998, EMBO J. 2:159-169).

An additional phenotypic assay has been described to define the activity of specific ATR inhibitory compounds is the cell cycle profile (P J Hurley, D Wilsker and F Bunz, Oncogene, 2007, 26, 2535-2542). Cells deficient in ATR have been shown to have defective cell cycle regulation and distinct characteristic profiles, particularly following a cytotoxic cellular insult. Furthermore, there are proposed to be differential responses between tumour and normal tissues in response to modulation of the ATR axis and this provides further potential for therapeutic intervention by ATR inhibitor molecules (Rodriguez-Bravo V et al, Cancer Res., 2007, 67, 11648-11656).

Another attractive utility of ATR-specific phenotypes is aligned with the concept of synthetic lethality and the observation that tumour cells that are deficient in G1 checkpoint controls, in particular p53 deficiency, are susceptible to inhibition of ATR activity resulting in premature chromatin condensation (PCC) and cell death (Ngheim et al, PNAS, 98, 9092-9097). In this situation, S-phase replication of DNA occurs but is not completed prior to M-phase initiation due to failure in the intervening checkpoints resulting in cell death from a lack of ATR signalling. The G2/M checkpoint is a key regulatory control involving ATR (Brown E. J. and Baltimore D., 2003, Genes Dev. 17, 615-628) and it is the compromise of this checkpoint and the prevention of ATR signalling to its downstream partners which results in PCC. Consequently, the genome of the daughter cells is compromised and viability of the cells is lost (Ngheim et al, PNAS, 98, 9092-9097).

In summary ATR inhibitors have the potential to sensitise tumour cells to ionising radiation or DNA-damage inducing chemotherapeutic agents, have the potential to induce selective tumour cell killing as well as to induce synthetic lethality in subsets of tumour cells with defects in DNA damage response.

In accordance with a first aspect of the present invention, there is provided a compound of formula (I):

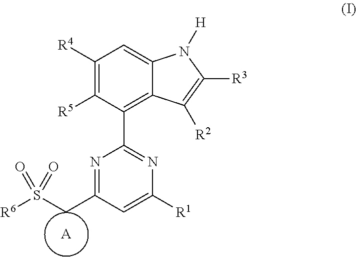

wherein:

Ring A is a C₃₋₆cycloalkyl or a saturated 4-6 membered heterocyclic ring containing one heteroatom selected from O, N and S;

R¹ is selected from morpholin-4-yl, 3-methylmorpholin-4-yl, 3,5-dimethylmorpholin-4-yl and a 8-oxa-3-azabicyclo[3.2.1]octan-3-yl group;

R² and R⁵ are hydrogen;

R³ is hydrogen or methyl;

R⁴ is selected from hydrogen, methyl, fluoro, chloro, cyano and methoxy; and

R⁶ is a group selected from methyl, ethyl, i-propyl and cyclopropyl;

or a pharmaceutically acceptable salt thereof.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention. Solvates and mixtures thereof also form an aspect of the present invention. For example, a suitable solvate of a compound of formula (I) is, for example, a hydrate such as a hemi-hydrate, a mono-hydrate, a di-hydrate or a tri-hydrate or an alternative quantity thereof. In another aspect of the present invention there is included compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

The present invention relates to the compounds of formula (I) as herein defined as well as to salts thereof. Salts for use in pharmaceutical compositions will be pharmaceutically acceptable salts, but other salts may be useful in the production of the compounds of formula (I) and their pharmaceutically acceptable salts. Pharmaceutically acceptable salts of the invention may, for example, include acid addition salts of compounds of formula (I) as herein defined which are sufficiently basic to form such salts. Such acid addition salts include but are not limited to furmarate, methanesulfonate, hydrochloride, hydrobromide, citrate and maleate salts and salts formed with phosphoric and sulfuric acid. In addition where compounds of formula (I) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of formula (I) may also be provided as in vivo hydrolysable esters. An in vivo hydrolysable ester of a compound of formula (I) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl, 1,3-dioxolen-2-onylmethyl esters for example 5-methyl-1,3-dioxolen-2-onylmethyl, and C₁₋₆alkoxycarbonyloxyethyl esters for example 1-methoxycarbonyloxyethyl; and may be formed at any carboxy group in the compounds of this invention.

Suitable pharmaceutically acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl, benzoyl, phenylacetyl, substituted benzoyl and phenylacetyl; C₁₋₁₀alkoxycarbonyl (to give alkyl carbonate esters), for example ethoxycarbonyl; di-C₁₋₄alkylcarbamoyl and N-(di-C₁₋₄alkylaminoethyl)-N—C₁₋₄alkylcarbamoyl (to give carbamates); di-C₁₋₄alkylaminoacetyl and carboxyacetyl. Examples of ring substituents on phenylacetyl and benzoyl include aminomethyl, C₁₋₄alkylaminomethyl and di-(C₁₋₄alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)OC₁₋₆alkyl-CO—, wherein R^{A} is for example, benzyloxy-C₁₋₄alkyl, or phenyl. Suitable substituents on a phenyl group in such esters include, for example, 4-C₁₋₄piperazino-C₁₋₄alkyl, piperazino-C₁₋₄alkyl and morpholino-C₁₋₄alkyl.

The compounds of the formula (I) may be also be administered in the form of a prodrug which is broken down in the human or animal body to give a compound of the formula (I). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);

### c) H. Bundgaard, Advanced Drug Delivery Reviews, 8, 1-38 (1992);

### d) H. Bundgaard, et al., Journal of Pharmaceutical Sciences, 77, 285 (1988); and

### e) N. Kakeya, et al., Chem Pharm Bull, 32, 692 (1984).

In this specification the generic term “C_{p-q}alkyl” includes both straight-chain and branched-chain alkyl groups. However references to individual alkyl groups such as “propyl” are specific for the straight chain version only (i.e. n-propyl and isopropyl) and references to individual branched-chain alkyl groups such as “tert-butyl” are specific for the branched chain version only.

The prefix C_{p-q} in C_{p-q}alkyl and other terms (where p and q are integers) indicates the range of carbon atoms that are present in the group, for example C₁₋₄alkyl includes C₁alkyl (methyl), C₂alkyl (ethyl), C₃alkyl (propyl as n-propyl and isopropyl) and C₄alkyl (n-butyl, sec-butyl, isobutyl and tert-butyl).

The term C_{p-q}alkoxy comprises —O—C_{p-q}alkyl groups.

The term C_{p-q}alkanoyl comprises —C(O)alkyl groups.

The term halo includes fluoro, chloro, bromo and iodo.

“Carbocyclyl” is a saturated, unsaturated or partially saturated monocyclic ring system containing from 3 to 6 ring atoms, wherein a ring CH₂ group may be replaced with a C═O group. “Carbocyclyl” includes “aryl”, “C_{p-q}cycloalkyl” and “C_{p-q}cycloalkenyl”.

“aryl” is an aromatic monocyclic carbocyclyl ring system.

“C_{p-q}cycloalkenyl” is an unsaturated or partially saturated monocyclic carbocyclyl ring system containing at least 1C═C bond and wherein a ring CH₂ group may be replaced with a C═O group.

“C_{p-q}cycloalkyl” is a saturated monocyclic carbocyclyl ring system and wherein a ring CH₂ group may be replaced with a C═O group.

“Heterocyclyl” is a saturated, unsaturated or partially saturated monocyclic ring system containing from 3 to 6 ring atoms of which 1, 2 or 3 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group. “Heterocyclyl” includes “heteroaryl”, “cycloheteroalkyl” and “cycloheteroalkenyl”.

“Heteroaryl” is an aromatic monocyclic heterocyclyl, particularly having 5 or 6 ring atoms, of which 1, 2 or 3 ring atoms are chosen from nitrogen, sulfur or oxygen where a ring nitrogen or sulfur may be oxidised.

“Cycloheteroalkenyl” is an unsaturated or partially saturated monocyclic heterocyclyl ring system, particularly having 5 or 6 ring atoms, of which 1, 2 or 3 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

“Cycloheteroalkyl” is a saturated monocyclic heterocyclic ring system, particularly having 5 or 6 ring atoms, of which 1, 2 or 3 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

This specification may make use of composite terms to describe groups comprising more than one functionality. Unless otherwise described herein, such terms are to be interpreted as is understood in the art. For example carbocyclylC_{p-q}alkyl comprises C_{p-q}alkyl substituted by carbocyclyl, heterocyclylC_{p-q}alkyl comprises C_{p-q}alkyl substituted by heterocyclyl, and bis(C_{p-q}alkyl)amino comprises amino substituted by 2 C_{p-q}alkyl groups which may be the same or different.

HaloC_{p-q}alkyl is a C_{p-q}alkyl group that is substituted by 1 or more halo substituents and particularly 1, 2 or 3 halo substituents. Similarly, other generic terms containing halo such as haloC_{p-q}alkoxy may contain 1 or more halo substituents and particularly 1, 2 or 3 halo substituents.

HydroxyC_{p-q}alkyl is a C_{p-q}alkyl group that is substituted by 1 or more hydroxyl substituents and particularly by 1, 2 or 3 hydroxy substituents. Similarly other generic terms containing hydroxy such as hydroxyC_{p-q}alkoxy may contain 1 or more and particularly 1, 2 or 3 hydroxy substituents.

C_{p-q}alkoxyC_{p-q}alkyl is a C_{p-q}alkyl group that is substituted by 1 or more C_{p-q}alkoxy substituents and particularly 1, 2 or 3 C_{p-q}alkoxy substituents. Similarly other generic terms containing C_{p-q}alkoxy such as C_{p-q}alkoxyC_{p-q}alkoxy may contain 1 or more C_{p-q}alkoxy substituents and particularly 1, 2 or 3 C_{p-q}alkoxy substituents.

Where optional substituents are chosen from “1 or 2”, from “1, 2, or 3” or from “1, 2, 3 or 4” groups or substituents it is to be understood that this definition includes all substituents being chosen from one of the specified groups i.e. all substitutents being the same or the substituents being chosen from two or more of the specified groups i.e. the substitutents not being the same.

Compounds of the present invention have been named with the aid of computer software (ACD/Name version 10.0).

“Proliferative disease(s)” includes malignant disease(s) such as cancer as well as non-malignant disease(s) such as inflammatory diseases, obstracutive airways diseases, immune diseases or cardiovascular diseases.

Suitable values for any R group or any part or substitutent for such groups include:
for C₁₋₃alkyl: methyl, ethyl, propyl and iso-propyl;
for C₁₋₆alkyl: C₁₋₃alkyl, butyl, 2-methylpropyl, tert-butyl, pentyl, 2,2-dimethylpropyl, 3-methylbutyl and hexyl;
for C₃₋₆cycloalkyl: cyclopropyl, cyclobutyl, cyclopentyl and cyclohexyl;
for C₃₋₆cycloalkylC₁₋₃ alkyl: cyclopropylmethyl, cyclopropylethyl, cyclobutylmethyl, cyclopentylmethyl and cyclohexylmethyl;
for aryl: phenyl;
for arylC₁₋₃ alkyl: benzyl and phenethyl;
for carbocylyl: aryl, cyclohexenyl and C₃₋₆cycloalkyl;
for halo: fluoro, chloro, bromo and iodo;
for C₁₋₃alkoxy: methoxy, ethoxy, propoxy and isopropoxy;
for C₁₋₆alkoxy: C₁₋₃alkoxy, butoxy, tert-butoxy, pentyloxy, 1-ethylpropoxy and hexyloxy;
for C₁₋₃alkanoyl: acetyl and propanoyl;
for C₁₋₆alkanoyl: acetyl, propanoyl and 2-methylpropanoyl;
for heteroaryl: pyridinyl, imidazolyl, pyrimidinyl, thienyl, pyrrolyl, pyrazolyl, thiazolyl, thiazolyl, triazolyl, oxazolyl, isoxazolyl, furanyl, pyridazinyl and pyrazinyl;
for heteroarylC₁₋₃ alkyl: pyrrolylmethyl, pyrrolylethyl, imidazolylmethyl, imidazolylethyl, pyrazolylmethyl, pyrazolylethyl, furanylmethyl, furanylethyl, thienylmethyl, theinylethyl, pyridinylmethyl, pyridinylethyl, pyrazinylmethyl, pyrazinylethyl, pyrimidinylmethyl, pyrimidinylethyl, pyrimidinylpropyl, pyrimidinylbutyl, imidazolylpropyl, imidazolylbutyl, 1,3,4-triazolylpropyl and oxazolylmethyl;
for heterocyclyl: heteroaryl, pyrrolidinyl, piperidinyl, piperazinyl, azetidinyl, morpholinyl, dihydro-2H-pyranyl, tetrahydropyridine and tetrahydrofuranyl;
for saturated heterocyclyl: oxetanyl, pyrrolidinyl, piperidinyl, piperazinyl, azetidinyl, morpholinyl, tetrahydropyranyl and tetrahydrofuranyl.

It should be noted that examples given for terms used in the description are not limiting.

Particular values of Ring A, n, R′, R², R³, R⁴, R⁵ and R⁶ are as follows. Such values may be used individually or in combination where appropriate, in connection with any aspect of the invention, or part thereof, and with any of the definitions, claims or embodiments defined herein.

Ring A

In one aspect of the invention Ring A is a unsubstituted C₃₋₆cycloalkyl or a saturated 4-6 heterocyclic ring containing one heteroatom selected from O and N

In another aspect Ring A is a cyclopropyl, cyclobutyl, cyclopentyl, oxetanyl, tetrahydrofuryl, tetrahydropyranyl, azetidinyl, pyrrolidinyl or piperidinyl ring.

In another aspect Ring A is a cyclopropyl, cyclobutyl, cylopentyl, tetrahydropyranyl or piperidinyl ring.

In another aspect Ring A is a cyclopropyl, cylopentyl, tetrahydropyranyl or piperidinyl ring.

In another aspect Ring A is a cyclopropyl, tetrahydropyranyl or piperidinyl ring.

In another aspect Ring A is a piperidinyl ring.

In another aspect Ring A is a tetrahydropyranyl ring.

In another aspect Ring A is a cyclopropyl ring.

R¹

In another aspect of the invention R¹ is selected from morpholin-4-yl, 3-methylmorpholin-4-yl, 3,5-dimethylmorpholin-4-yl and a 8-oxa-3-azabicyclo[3.2.1]octan-3-yl.

In a further aspect, R¹ is selected from morpholin-4-yl, 3-methylmorpholin-4-yl.

In a further aspect, R¹ is 3-methylmorpholin-4-yl.

R²

R² is hydrogen.

R³

In one aspect of the invention R³ is hydrogen.

R⁴

In another aspect R⁴ is selected from hydrogen, fluoro, chloro, cyano, methyl and methoxy.

In another aspect R⁴ is hydrogen or methyl.

In another aspect R⁴ is hydrogen.

R⁵

In one aspect of the invention R⁵ is hydrogen.

R⁶

In one aspect of the invention R⁶ is a group selected from methyl, ethyl, i-propyl and cyclopropyl.

In another aspect of the invention R⁶ is methyl.

In one aspect of the invention there is provided a subset of compounds of formula (I), or a pharmaceutically acceptable salt thereof;

Ring A is a unsubstituted C₃₋₆cycloalkyl or a saturated 4-6 heterocyclic ring containing one heteroatom selected from O and N;

R¹ is selected from morpholin-4-yl, 3-methylmorpholin-4-yl, 3,5-dimethylmorpholin-4-yl and a 8-oxa-3-azabicyclo[3.2.1]octan-3-yl group;

R² is hydrogen;

R⁵ is hydrogen;

R⁶ is a group selected from methyl, ethyl, i-propyl and cyclopropyl; and either

R³ is hydrogen; and R⁴ is selected from hydrogen, methyl, fluoro, chloro, cyano and methoxy;

or

R⁴ is hydrogen, and R³ is hydrogen or methyl.

In another aspect of the invention there is provided a subset of compounds of formula (I), or a pharmaceutically acceptable salt thereof;

Ring A is a cyclopropyl, cyclobutyl, cyclopentyl, oxetanyl, tetrahydrofuryl, tetrahydropyranyl, azetidinyl, pyrrolidinyl or piperidinyl ring;

R¹ is selected from morpholin-4-yl, 3-methylmorpholin-4-yl, 3,5-dimethylmorpholin-4-yl and a 8-oxa-3-azabicyclo[3.2.1]octan-3-yl group;

R² is hydrogen;

R⁵ is hydrogen; and

R⁶ is a group selected from methyl, ethyl, i-propyl and cyclopropyl; and either

R³ is hydrogen; and R⁴ is selected from hydrogen, methyl, fluoro, chloro, cyano and methoxy;

or

R⁴ is hydrogen, and R³ is hydrogen or methyl.

In another aspect of the invention there is provided a subset of compounds of formula (I), or a pharmaceutically acceptable salt thereof;

Ring A is a cyclopropyl, cylopentyl, tetrahydropyranyl or piperidinyl ring;

R¹ is selected from morpholin-4-yl, 3-methylmorpholin-4-yl, 3,5-dimethylmorpholin-4-yl and a 8-oxa-3-azabicyclo[3.2.1]octan-3-yl group;

R² is hydrogen;

R⁵ is hydrogen; and

R⁶ is a group selected from methyl, ethyl, i-propyl and cyclopropyl; and

either

R³ is hydrogen; and R⁴ is selected from hydrogen, methyl, fluoro, chloro, cyano and methoxy;

or

R⁴ is hydrogen, and R³ is hydrogen or methyl.

In another aspect of the invention there is provided a subset of compounds of formula (I), or a pharmaceutically acceptable salt thereof;

Ring A is a cyclopropyl, cyclobutyl, cyclopentyl, oxetanyl, tetrahydrofuryl, tetrahydropyranyl, azetidinyl, pyrrolidinyl or piperidinyl ring;

R¹ is selected from morpholin-4-yl, 3-methylmorpholin-4-yl, 3,5-dimethylmorpholin-4-yl and a 8-oxa-3-azabicyclo[3.2.1]octan-3-yl group;

R² is hydrogen;

R³ is hydrogen;

R⁴ is hydrogen;

R⁵ is hydrogen; and

R⁶ is a group selected from methyl, ethyl, i-propyl and cyclopropyl.

In another aspect of the invention there is provided a subset of compounds of formula (I), or a pharmaceutically acceptable salt thereof;

Ring A is a cyclopropyl, cyclobutyl, cyclopentyl, tetrahydropyranyl or piperidinyl ring;

R¹ is selected from morpholin-4-yl, 3-methylmorpholin-4-yl, 3,5-dimethylmorpholin-4-yl and a 8-oxa-3-azabicyclo[3.2.1]octan-3-yl group;

R² is hydrogen;

R³ is hydrogen;

R⁴ is hydrogen;

R⁵ is hydrogen; and

R⁶ is a group selected from methyl, ethyl, i-propyl and cyclopropyl.

In another aspect of the invention there is provided a subset of compounds of formula (I), or a pharmaceutically acceptable salt thereof;

n is 0 or 1;

Ring A is a cyclopropyl, cylopentyl, tetrahydropyranyl or piperidinyl ring;

R¹ is selected from morpholin-4-yl, 3-methylmorpholin-4-yl;

R² is hydrogen;

R³ is hydrogen;

R⁴ is methyl;

R⁵ is hydrogen; and

R⁶ is a group selected from methyl and cyclopropyl.

In another aspect of the invention there is provided a subset of compounds of formula (I), or a pharmaceutically acceptable salt thereof;

Ring A is a cyclopropyl, cyclobutyl, cyclopentyl, tetrahydropyranyl or piperidinyl ring;

R¹ is 3-methylmorpholin-4-yl;

R² is hydrogen;

R³ is hydrogen;

R⁴ is hydrogen;

R⁵ is hydrogen; and

R⁶ is a group selected from methyl, ethyl, i-propyl and cyclopropyl.

In another aspect of the invention there is provided a subset of compounds of formula (I), or a pharmaceutically acceptable salt thereof;

Ring A is a cyclopropyl, tetrahydropyranyl or piperidinyl ring;

R¹ is 3-methylmorpholin-4-yl;

R² is hydrogen;

R³ is hydrogen;

R⁴ is hydrogen;

R⁵ is hydrogen; and

R⁶ is a methyl group.

In another aspect of the invention there is provided a subset of compounds of formula (Ia),

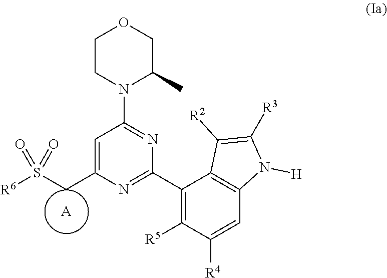

or a pharmaceutically acceptable salt thereof;

Ring A is a cyclopropyl, tetrahydropyranyl or piperidinyl ring;

R² is hydrogen;

R³ is hydrogen;

R⁴ is hydrogen;

R⁵ is hydrogen; and

R⁶ is a methyl group.

In another aspect of the invention provides a compound, or a combination of compounds, selected from any one of the Examples or a pharmaceutically acceptable salt thereof.

In another aspect of the invention there is provided a compound, or a combination of compounds, selected from any one of
4-{4-[1-(Methylsulfonyl)cyclopropyl]-6-morpholin-4-ylpyrimidin-2-yl}-1H-indole;
6-Methyl-4-{4-[1-(methylsulfonyl)cyclopropyl]-6-morpholin-4-ylpyrimidin-2-yl}-1H-indole;
2-Methyl-4-{4-[1-(methylsulfonyl)cyclopropyl]-6-morpholin-4-ylpyrimidin-2-yl}-1H-indole;
6-Methoxy-4-{4-[1-(methylsulfonyl)cyclopropyl]-6-morpholin-4-ylpyrimidin-2-yl}-1H-indole;
4-{4-[1-(methylsulfonyl)cyclopropyl]-6-morpholin-4-ylpyrimidin-2-yl}-1H-indole-6-carbonitrile;
6-chloro-4-{4-[1-(methylsulfonyl)cyclopropyl]-6-morpholin-4-ylpyrimidin-2-yl}-1H-indole;
6-fluoro-4-{4-[1-(methylsulfonyl)cyclopropyl]-6-morpholin-4-ylpyrimidin-2-yl}-1H-indole;
4-{4-[4-(Methylsulfonyl)piperidin-4-yl]-6-morpholin-4-ylpyrimidin-2-yl}-1H-indole;
4-{4-[4-(Methylsulfonyl)tetrahydro-2H-pyran-4-yl]-6-morpholin-4-ylpyrimidin-2-yl}-1H-indole;
4-{4-[1-(ethylsulfonyl)cyclopropyl]-6-morpholin-4-ylpyrimidin-2-yl}-1H-indole;
4-(4-{1-[(1-methylethyl)sulfonyl]cyclopropyl}-6-morpholin-4-ylpyrimidin-2-yl)-1H-indole;
4-{4-[1-(cyclopropylsulfonyl)cyclopropyl]-6-morpholin-4-ylpyrimidin-2-yl}-1H-indole;
4-{4-[4-(cyclopropylsulfonyl)tetrahydro-2H-pyran-4-yl]-6-morpholin-4-ylpyrimidin-2-yl}-1H-indole;
4-{4-[4-(cyclopropylsulfonyl)piperidin-4-yl]-6-morpholin-4-ylpyrimidin-2-yl}-1H-indole;
4-{4-[4-(Cyclopropylsulfonyl)piperidin-4-yl]-6-[(3S)-3-methylmorpholin-4-yl]pyrimidin-2-yl}-1H-indole;
4-{4-[4-(Cyclopropylsulfonyl)tetrahydro-2H-pyran-4-yl]-6-[(3S)-3-methylmorpholin-4-yl]pyrimidin-2-yl}-1H-indole;
4-{4-[(3S)-3-Methylmorpholin-4-yl]-6-[1-(methylsulfonyl)cyclopentyl]pyrimidin-2-yl}-1H-indole;
4-{4-[(3S)-3-Methylmorpholin-4-yl]-6-[4-(methylsulfonyl)tetrahydro-2H-pyran-4-yl]pyrimidin-2-yl}-1H-indole;
4-{4-[(3S)-3-Methylmorpholin-4-yl]-6-[1-(methylsulfonyl)-cyclopropyl]pyrimidin-2-yl}-1H-indole;
4-{4-[(3R)-3-Methylmorpholin-4-yl]-6-[1-(methylsulfonyl)cyclopropyl]pyrimidin-2-yl}-1H-indole;
6-Methyl-4-{4-[(3R)-3-methylmorpholin-4-yl]-6-[1-(methylsulfonyl)cyclopropyl]pyrimidin-2-yl}-1H-indole;
2-Methyl-4-{4-[(3R)-3-methylmorpholin-4-yl]-6-[1-(methylsulfonyl)cyclopropyl]pyrimidin-2-yl}-1H-indole;
4-{4-[(3R)-3-Methylmorpholin-4-yl]-6-[1-(methylsulfonyl)cyclopropyl]pyrimidin-2-yl}-1H-indole-6-carbonitrile;
6-chloro-4-{4-[(3R)-3-methylmorpholin-4-yl]-6-[1-(methylsulfonyl)cyclopropyl]pyrimidin-2-yl}-1H-indole;
6-fluoro-4-{4-[(3R)-3-methylmorpholin-4-yl]-6-[1-(methylsulfonyl)cyclopropyl]pyrimidin-2-yl}-1H-indole;
6-methoxy-4-{4-[(3R)-3-methylmorpholin-4-yl]-6-[1-(methylsulfonyl)cyclopropyl]pyrimidin-2-yl}-1H-indole;
4-{4-[(3R)-3-Methylmorpholin-4-yl]-6-[4-(methylsulfonyl)tetrahydro-2H-pyran-4-yl]pyrimidin-2-yl}-1H-indole;
6-Methyl-4-{4-[(3R)-3-methylmorpholin-4-yl]-6-[4-(methylsulfonyl)tetrahydro-2H-pyran-4-yl]pyrimidin-2-yl}-1H-indole;
2-Methyl-4-{4-[(3R)-3-methylmorpholin-4-yl]-6-[4-(methylsulfonyl)tetrahydro-2H-pyran-4-yl]pyrimidin-2-yl}-1H-indole;
6-Methoxy-4-{4-[(3R)-3-methylmorpholin-4-yl]-6-[4-(methylsulfonyl)tetrahydro-2H-pyran-4-yl]pyrimidin-2-yl}-1H-indole;
6-chloro-4-{4-[(3R)-3-methylmorpholin-4-yl]-6-[4-(methylsulfonyl)tetrahydro-2H-pyran-4-yl]pyrimidin-2-yl}-1H-indole;
6-fluoro-4-{4-[(3R)-3-methylmorpholin-4-yl]-6-[4-(methylsulfonyl)tetrahydro-2H-pyran-4-yl]pyrimidin-2-yl}-1H-indole;
4-{4-[(3R)-3-methylmorpholin-4-yl]-6-[4-(methylsulfonyl)tetrahydro-2H-pyran-4-yl]pyrimidin-2-yl}-1H-indole-6-carbonitrile;
4-{4-[(3R)-3-Methylmorpholin-4-yl]-6-[4-(methylsulfonyl)piperidin-4-yl]pyrimidin-2-yl}-1H-indole;
4-{4-[(3R)-3-Methylmorpholin-4-yl]-6-[1-(methylsulfonyl)cyclobutyl]pyrimidin-2-yl}-1H-indole;
4-{4-[1-(Cyclopropylsulfonyl)cyclopropyl]-6-[(3R)-3-methylmorpholin-4-yl]pyrimidin-2-yl}-1H-indole;
4-{4-[4-(Cyclopropylsulfonyl)piperidin-4-yl]-6-[(3R)-3-methylmorpholin-4-yl]pyrimidin-2-yl}-1H-indole;
4-{4-[4-(Cyclopropylsulfonyl)tetrahydro-2H-pyran-4-yl]-6-[(3R)-3-methylmorpholin-4-yl]pyrimidin-2-yl}-1H-indole;
4-{4-[1-(Ethylsulfonyl)cyclopropyl]-6-[(3R)-3-methylmorpholin-4-yl]pyrimidin-2-yl}-1H-indole;
4-{4-[4-(Ethylsulfonyl)tetrahydro-2H-pyran-4-yl]-6-[(3R)-3-methylmorpholin-4-yl]pyrimidin-2-yl}-1H-indole;
4-{4-[4-(Ethylsulfonyl)piperidin-4-yl]-6-[(3R)-3-methylmorpholin-4-yl]pyrimidin-2-yl}-1H-indole;
4-(4-{1-[(1-Methylethyl)sulfonyl]cyclopropyl}-6-[(3R)-3-methylmorpholin-4-yl]pyrimidin-2-yl)-1H-indole;
4-(4-{4-[(1-Methylethyl)sulfonyl]tetrahydro-2H-pyran-4-yl}-6-[(3R)-3-methylmorpholin-4-yl]pyrimidin-2-yl)-1H-indole;
4-(4-{4-[(1-Methylethyl)sulfonyl]piperidin-4-yl]-6-[(3R)-3-methylmorpholin-4-yl]pyrimidin-2-yl}-1H-indole;
4-{4-[(3S,5R)-3,5-Dimethylmorpholin-4-yl]-6-[1-(methylsulfonyl)cyclopropyl]pyrimidin-2-yl}-1H-indole;
4-[4-[(3R,5S)-3,5-dimethylmorpholin-4-yl]-6-(1-methylsulfonylcyclopropyl)pyrimidin-2-yl]-1H-indole); and
4-{4-[1-(Methylsulfonyl)cyclopropyl]-6-(8-oxa-3-azabicyclo[3.2.1]oct-3-yl)pyrimidin-2-yl}-1H-indole, or a pharmaceutically acceptable salt thereof.

A compound of formula (I) may be prepared from a compound of formula (II), wherein L² is a leaving group (such as halo or —SMe, etc.), with a compound of formula (III), wherein X is a suitable group (such as boronic acid or ester) in the presence of a suitable Pd catalyst and phosphine ligand in a suitable solvent such as a mixture of dimethylformamide, dimethoxyethane, water and ethanol, under suitable conditions such as heating in a microwave reactor.

Compounds of formula (III) are either commercially available or well known in the art.

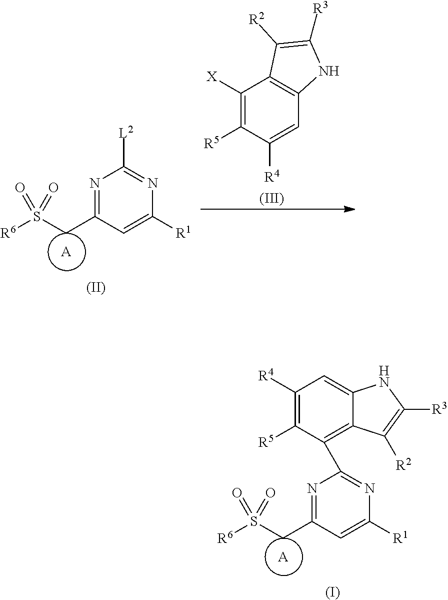

It will be appreciated that a compound of formula (II) may be transformed into another compound of formula (II) by techniques such as oxidation, alkylation, reductive amination etc., either listed above or otherwise known in the literature.

A compound of formula (II), may be prepared by the reaction of a compound of formula (IV), with a compound of formula (V), wherein A is a 2 to 6 membered, optionally substituted, alkylene chain in which 1 carbon may be optionally replaced with O, N or S, and wherein L¹ is a leaving group (such as halo, tosyl, mesyl etc.), in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

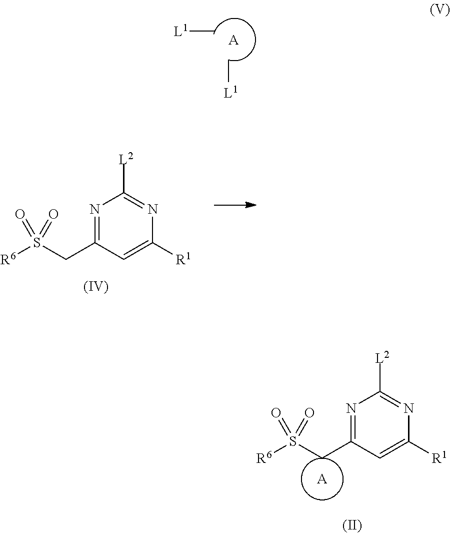

A compound of formula (II), may be prepared by the reaction of a compound of formula (IV), with a compound of formula (V), wherein A is a 2 to 6 membered, optionally substituted, alkylene chain in which 1 carbon may be optionally replaced with O, N or S, and wherein L¹ is a leaving group (such as halo, tosyl, mesyl etc.), and L³ is a group which can be transformed to a suitable leaving group (such as halo, tosyl, mesyl) at a later stage, to give a compound of formula (VI) in the presence of a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide, and subsequently converting L³ to an appropriate leaving group (such as halo, tosyl, mesyl etc.) and then exposing to a suitable base such as sodium hydride or potassium tert-butoxide in a suitable solvent such as tetrahydrofuran or N,N-dimethylformamide, or by using aqueous sodium hydroxide solution and DCM as a solvent with a suitable phase transfer agent such as tetrabutylammonium bromide.

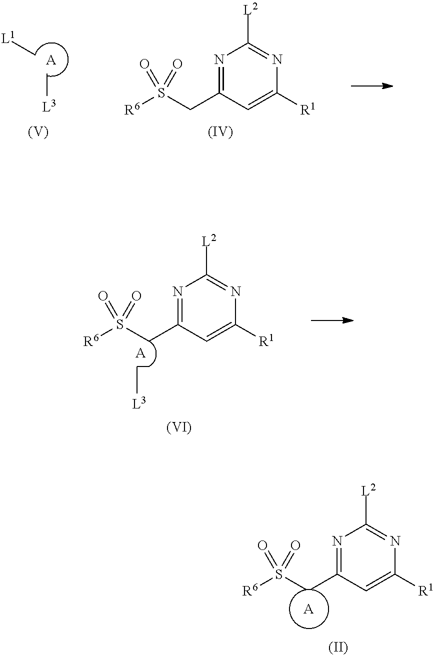

A compound of formula (IV), may be prepared by the reaction of a compound of formula (VII) where L⁴ is a suitable leaving group such as halo, with a compound of formula (VIII) in a suitable solvent such as DCM.

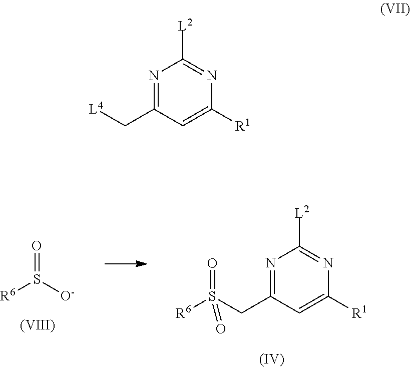

A compound of formula (VII), may be prepared by the reaction of a compound of formula (IX) using conditions well known in the art.

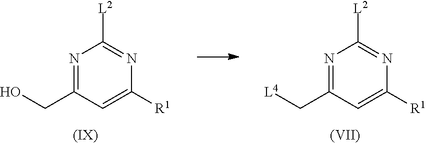

A compound of formula (IX), may be prepared by the reaction of a compound of formula (X) using conditions well known in the art.

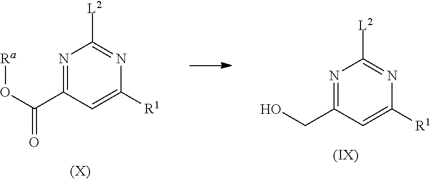

A compound of formula (X), where R¹ is a N-linked heterocycle such as morpholine, may be prepared by the reaction of a compound of formula (XI) with a cyclic amine such as morpholine optionally in the presence of a suitable base such as triethylamine in a suitable solvent such as N,N-dimethylformamide. A compound of formula (X), where R¹ is a C-linked heterocycle such as dihydropyran, may be prepared by the reaction of a compound of formula (XI) with a suitable organometallic reagent (such as the boronic acid R′B(OH)₂ or the boronic ester R′B(OR)₂ etc.) in the presence of a suitable metal catalyst (such as palladium or copper) in a suitable solvent such as 1,4-dioxane.

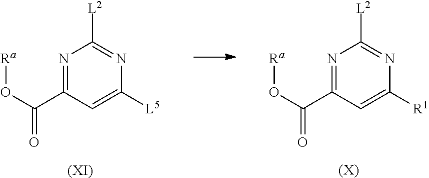

Compounds of formula (XI), cyclic amines, boronic acids {R¹B(OH)₂}and boronic esters {R¹B(OR)₂}are either commercially available or well known in the art.

A compound of formula (IV) where R¹ is a N-linked heterocycle such as morpholine, may be prepared by the reaction of a compound of formula (XII) with a cyclic amine such as morpholine optionally in the presence of a suitable base such as triethylamine in a suitable solvent such as N,N-dimethylformamide. A compound of formula (VI), where R¹ is a C-linked heterocycle such as dihydropyran, may be prepared by the reaction of a compound of formula (XII) with a suitable organometallic reagent (such as the boronic acid R′B(OH)₂ or the boronic ester R′B(OR)₂ etc.) in the presence of a suitable metal catalyst (such as palladium or copper) in a suitable solvent such as 1,4-dioxane.

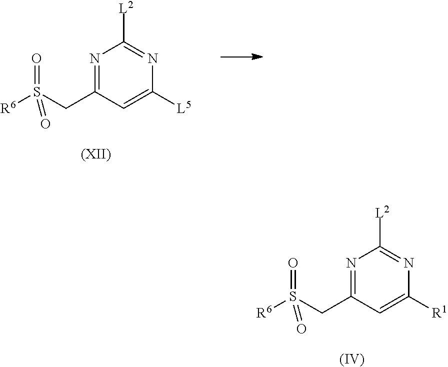

A compound of formula (XII), may be prepared by the reaction of a compound of formula (XIII) under conditions known in the art.

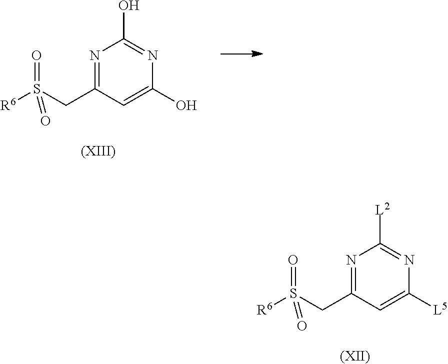

A compound of formula (XIII), may be prepared by the reaction of a compound of formula (XIV) with a compound of formula (VIII).

Alternatively, a compound of formula (XII) where R⁶ is Me, L² is —SMe and L⁵ is chloro, may be prepared by the reaction of a compound of formula (XIV) with a compound of formula (XV), followed by decarboxylation under conditions known in the art.

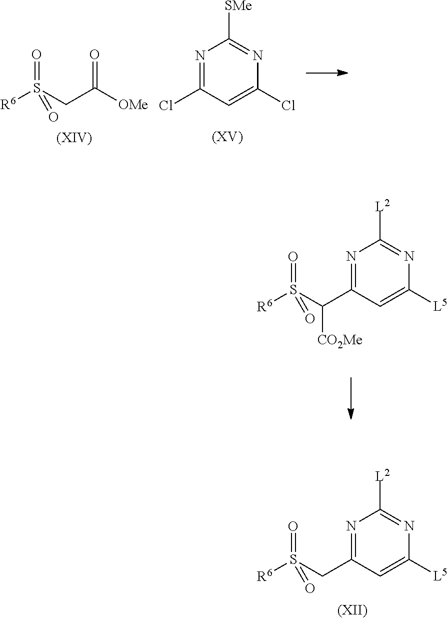

A compound of formula (IV), may be prepared by the reaction of a compound of formula (XVI) under suitable oxidation conditions such as aqueous hydrogen peroxide in the presence of sodium tungstate dihydrate in a suitable solvent mixture such as methanol and 1,4-dioxane in the presence of water and sulfuric acid.

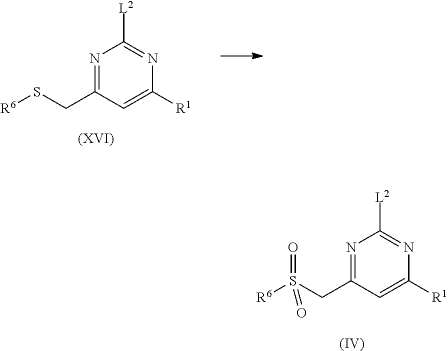

It will be appreciated that a compound of formula (IV) may be transformed into another compound of formula (IV) by techniques such as oxidation, alkylation, reductive amination etc., either listed above or otherwise known in the literature.

A compound of formula (XVI), may be prepared by the reaction of a compound of formula (VII), wherein L⁴ is a leaving group (such as halo, tosyl, mesyl etc), with a compound of formula (XV) optionally in the presence of a suitable base such as triethylamine and a solvent such as acetonitrile.

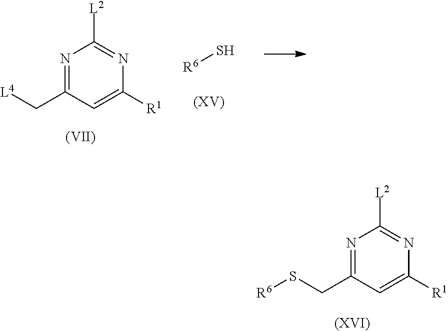

Compounds of formula (VII) are either commercially available or well known in the art.

It will be appreciated that a compound of formula (XVI) may be transformed into another compound of formula (XVI) by techniques such as oxidation, alkylation, reductive amination etc., either listed above or otherwise known in the literature.

It will be appreciated that where Ring A, is a heterocyclic ring containing a nitrogen atom that the nitrogen atom may be suitably protected (for example a t-butoxycarbamate or benzyl group) and that the protecting group may be removed and if necessary a further reaction performed on the nitrogen (for example an alkylation, reductive amination or amidation) at any stage in the synthesis.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. For example compounds of formula (I) may be converted into further compounds of formula (I) by standard aromatic substitution reactions or by conventional functional group modifications. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Many of the intermediates defined herein are novel and these are provided as a further feature of the invention.

### Biological Assays

The following assays can be used to measure the effects of the compounds of the present invention as ATR kinase inhibitors.

(a) Enzyme Assay—ATR

ATR for use in the in vitro enzyme assay was obtained from HeLa nuclear extract (CIL Biotech, Mons, Belgium) by immunoprecipitation with rabbit polyclonal antiserum raised to amino acids 400-480 of ATR (Tibbetts R S et al, 1999, Genes Dev. 13:152-157) contained in the following buffer (25 mM HEPES (pH7.4), 2 mM MgCl₂, 250 mM NaCl, 0.5 mM EDTA, 0.1 mM Na₃VO₄, 10% v/v glycerol, and 0.01% v/v Tween 20). ATR-antibody complexes were isolated from nuclear extract by incubating with protein A-Sepharose beads (Sigma, #P3476) for 2 hours and then through centrifugation to recover the beads. In the well of a 96-well plate, 10 μL ATR-containing Sepharose beads were incubated with 1 μg of substrate glutathione S-transferase-p53N66 (NH₂-terminal 66 amino acids of p53 fused to glutathione S-transferase was expressed in E. coli) in ATR assay buffer (50 mM HEPES (pH 7.4), 150 mM NaCl, 6 mM MgCl₂, 4 mM MnCl₂, 0.1 mM Na₃VO₄, 0.1 mM DTT, and 10% (v/v) glycerol) at 37° C. in the presence or absence of inhibitor. After 5 minutes with gentle shaking, ATP was added to a final concentration of 3 μM and the reaction continued at 37° C. for an additional 1 hour. The reaction was stopped by addition of 100 μL PBS and the reaction was transferred to a white opaque glutathione coated 96-well plate (NUNC #436033) and incubated overnight at 4° C. This plate was then washed with PBS/0.05% (v/v) Tween 20, blotted dry, and analyzed by a standard ELISA (Enzyme-Linked ImmunoSorbent Assay) technique with a phospho-serine 15 p53 (16G8) antibody (Cell Signaling Technology, #9286). The detection of phosphorylated glutathione S-transferase-p53N66 substrate was performed in combination with a goat anti-mouse horseradish peroxidase-conjugated secondary antibody (Pierce, #31430). Enhanced chemiluminescence solution (NEN, Boston, Mass.) was used to produce a signal and chemiluminescent detection was carried out via a TopCount (Packard, Meriden, Conn.) plate reader.

The resulting calculated % enzyme activity (Activity Base, IDBS) was then used to determine the IC₅₀ values for the compounds (IC₅₀ taken as the concentration at which 50% of the enzyme activity is inhibited).

(b) Cellular Assays—ATR (Western Blot)

The ATR western blot assay was used to determine the ability of the ATR inhibitor to prevent the phosphorylation of Chk1 at serine 345 in response to treatment with the UV mimetic 4-nitroquiniline N-oxide (4NQO).

HT29 colorectal adenocarcinoma cells (ATCC) were routinely maintained in RPMI (Invitrogen) supplemented with 10% fetal bovine serum and penicillin/streptomycin/glutamine at 37° C. and 5% CO₂ in a humidified atmosphere. The cells were seeded at 5×10⁵ cells per well in 6-well tissue culture treated dishes. Cells were left overnight before being treated with a range of concentrations of ATR inhibitor (typically 10, 3, 1, 0.3, 0.1 0.03 μM) for 1 hour at 37° C. Cells were then treated for a further hour with 3 μM 4NQO (Sigma #N8141), controls containing 4NQO alone and equivalent DMSO were also included. Whole cell lysates were prepared by scraping cells into 50 ul lysis buffer (1% NP-40, 5 mM NaF, 1 mM NaVO₄, in PBS plus EDTA-free protease inhibitor tablet (Roche)). Lysates were spun to remove insoluable material, SDS PAGE loading buffer added and boiled. The proteins were separated on 10% Bis-Tris SDS-PAGE gels (Invitrogen) and transferred to nitrocelluloseusing Biorad Mini Protean II apparatus. Chk1 (ser 345) was detected using anti phospho Chk1 (ser 345) 133D3 Rabbit Mab (Cell Signalling Technology #2345) at 1:1000 dilution in 5% BSA, and goat anti rabbit IgG HRP conjugated secondary antibody (Pierce #31460) in 10% Marvel followed by detection using Enhanced Chemilumiescence Solution (Perkin Elmer).

(c) Cellular Assays—ATR

ATM and ATR have distinct and overlapping responses to DNA damage. They must participate together and responses must be co-ordinated. Both pathways may be activated by ionising radiation, however only ATR is activated by UV. Since UV treatment is not practical for use in a high throughput cell assay, the UV mimetic 4NQ0 (Sigma) was chosen to activate the ATR DNA damage response pathway.

Chk1, a downstream protein kinase of ATR, plays a key role in DNA damage checkpoint control. Activation of Chk1 involves phosphorylation of Ser317 and Ser345 (regarded as the preferential target for phosphorylation/activation by ATR). This assay measures a decrease in phosphorylation of Chk1 (Ser 345) in HT29 colon adenocarcinoma cells following treatment with compound and the UV mimetic 4NQ0. Compounds dose ranges were created by diluting in 100% DMSO and then further into assay media (EMEM, 10% FCS, 1% glutamine) using a Labcyte Echo Acoustic dispensing instrument. Cells were plated in 384 well Costar plates at 1×10⁵ cells per ml in 40 μL EMEM, 10% FCS, 1% glutamine and grown for 24 hrs. Following addition of compound the cells were incubated for 60 minutes. A final concentration of 3 μM 4NQ0 (prepared in 100% DMSO) was then added using the Labcyte Echo and the cells incubated for a further 60 mins. The cells are then fixed by adding 40 μL 3.7% v/v formaldehyde solution for 20 minutes. After removal of fix, cells were washed 3 times with PBS and permeabilised in 40 μL of PBS containing 0.1% Triton™ X-100. Cells are then washed three times and 15 μl primary antibody solution (pChk1 Ser345) (Cell Signalling Technology #2345) added and the plates incubated at 4° C. overnight. The primary antibody is then washed off, and 20 μl secondary antibody solution (goat anti-rabbit Alexa Fluor 488, Invitrogen) and 1 μM Hoechst 33258 (Invitrogen) is added for 90 mins at room temperature. The plates are washed twice and left in 40 μl PBS. Plates were then read on an ArrayScan Vti instrument to determine staining intensities, and dose responses were obtained and used to determine the IC₅₀ values for the compounds. Specifically, the ratio of nuclear to cytoplasmic staining was measured and used to monitor the reduction in strong nuclear phospho-Chk1 (S345) staining observed upon ATR inhibition.

(d) Cellular—SRB Assay

The potentiation factor (PF₅₀) for compounds is a measure of the fold increase in effect of a chemotherapeutic agent, when used in combination with an ATR inhibitor. Specifically, this is calculated as a ratio of the IC₅₀ of control cell growth in the presence of a chemotherapeutic agent, typically carboplatin, divided by the IC₅₀ of cell growth in the presence of this agent and the ATR inhibitor of interest. For this purpose, HT29 cells were seeded at the appropriate density to ensure exponential growth throughout the time of the assay (typically 1000-1500 cells) in each well of a 96-well plate, in a volume of 80 μl and incubated overnight at 37° C. Subsequently, cells were dosed with either DMSO vehicle, or treated with test compounds at fixed concentrations (typically 1, 0.3 & 0.1 μM). Following a one hour incubation at 37° C., the cells were further treated with a 10 point dose response of the chemotherapeutic agent, based on it's known sensitivity (typically 30-0.001 ug/ml for carboplatin). Cells were left to grow for 5 days at 37° C., after which time cell growth was assessed using the sulforhodamine B (SRB) assay (Skehan, P et al, 1990 New colorimetric cytotoxic assay for anticancer-drug screening. J. Natl. Cancer Inst. 82, 1107-1112.). Specifically, the media was removed and cells fixed with 100 μl of ice cold 10% (w/v) trichloroacetic acid. The plates were then incubated at 4° C. for 20 minutes prior to washing 4 times with water. Each well was then stained with 100 μL of 0.4% (w/v) SRB in 1% acetic acid for 20 minutes before a further 4 washes with 1% acetic acid. Plates were then dried for 2 hours at room temperature and the dye was solubilized by the addition of 100 μL Tris Base pH 8.5 into each well. Plates were shaken before measuring optical density at 564 nm (OD₅₆₄). In order to calculate the PF50, the OD₅₆₄ values obtained for the dose-response curve of chemotherapeutic agent were expressed as a percentage of the value obtained from cells treated with vehicle alone. Similarly, to act as a control for inclusion of the ATR inhibitor, values from the chemotherapeutic agent tested in combination with a fixed ATR inhibitor concentration were expressed as a percentage of the value obtained from cells treated with the corresponding concentration of ATR inhibitor alone. From these internally-controlled curves, IC50 values were calculated and the PF50 was determined as the ratio of these values, as described above. Compounds are compared using the PF50 value at concentrations of ATR inhibitor that show minimal growth inhibition on their own.

The following assays can be used to measure the effects of the compounds of the present invention as mTOR kinase inhibitors.

Enzyme—mTOR Kinase Assay (1)

mTOR protein was immunopurified from HeLa cytoplasmic extract (Cilbiotech, #CC-01-40-50) using an anti-mTOR rabbit serum. Immunopurified mTOR was then used in a kinase assay to phosphorylate the mTOR substrate PHAS-1 in the presence of ATP and the addition of test compounds. Wild type and mutant His-PHAS-1 (T37A/T46A) proteins were expressed in E. coli (BL21-RIL) from a pET15b expression vector. Proteins were affinity purified on Ni-agarose (Qiagen, #30210). Varying concentrations of compounds or DMSO were added to a 96 well v-bottomed assay plate (Greiner, #651201), then 32.5 μL of kinase buffer (10 mM Hepes pH7.5; 50 mM NaCl; 10 mM MgCl₂; 10 mM MnCl₂; 1 mM DTT; 0.1 mM Na-orthovanadate) containing 1.2 μg/μL of PHAS-1 protein was added to the compounds. Finally 10 μL of immunoprecipitated mTOR enzyme in kinase buffer was added to the plate. Enzyme and substrate were incubated for 10 minutes at 37° C. before addition of 50 μM ATP and reactions were allowed to proceed for one hour at 30° C.

An ELISA (Enzyme-Linked ImmunoSorbent Assay) was employed to quantify the phosphorylated Thr-37/46 on PHAS-1 substrate using an HRP conjugated antibody to produce a chemiluminescent end-point which can be measured. Enhanced chemiluminescence solution (NEN, Boston, Mass.) was used to produce a signal and chemiluminescent detection was carried out via a TopCount (Packard, Meriden, Conn.) plate reader.

Enzyme—mTOR Kinase Assay (2)

The assay used AlphaScreen technology (Gray et al., Analytical Biochemistry, 2003, 313: 234-245) to determine the ability of test compounds to inhibit phosphorylation by recombinant mTOR.

A C-terminal truncation of mTOR encompassing amino acid residues 1362 to 2549 of mTOR (EMBL Accession No. L34075) was stably expressed as a FLAG-tagged fusion in HEK293 cells as described by Vilella-Bach et al., Journal of Biochemistry, 1999, 274, 4266-4272. The HEK293 FLAG-tagged mTOR (1362-2549) stable cell line was routinely maintained at 37° C. with 5% CO₂ up to a confluency of 70-90% in Dulbecco's modified Eagle's growth medium (DMEM; Invitrogen Limited, Paisley, UK Catalogue No. 41966-029) containing 10% heat-inactivated foetal calf serum (FCS; Sigma, Poole, Dorset, UK, Catalogue No. F0392), 1% L-glutamine (Gibco, Catalogue No. 25030-024) and 2 mg/ml Geneticin (G418 sulfate; Invitrogen Limited, UK Catalogue No. 10131-027). Following expression in the mammalian HEK293 cell line, expressed protein was purified using the FLAG epitope tag using standard purification techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one). A 30 μl mixture of recombinant purified mTOR enzyme, 1 μM biotinylated peptide substrate (Biotin-Ahx-Lys-Lys-Ala-Asn-Gln-Val-Phe-Leu-Gly-Phe-Thr-Tyr-Val-Ala-Pro-Ser-Val-Leu-Glu-Ser-Val-Lys-Glu-NH₂; Bachem UK Ltd), ATP (20 μM) and a buffer solution [comprising Tris-HCl pH7.4 buffer (50 mM), EGTA (0.1 mM), bovine serum albumin (0.5 mg/mL), DTT (1.25 mM) and manganese chloride (10 mM)] was agitated at room temperature for 90 minutes.

Control wells that produced a maximum signal corresponding to maximum enzyme activity were created by using 5% DMSO instead of test compound. Control wells that produced a minimum signal corresponding to fully inhibited enzyme were created by adding EDTA (83 mM) instead of test compound. These assay solutions were incubated for 2 hours at room temperature.

Each reaction was stopped by the addition of 10 μl of a mixture of EDTA (50 mM), bovine serum albumin (BSA; 0.5 mg/mL) and Tris-HCl pH7.4 buffer (50 mM) containing p70 S6 Kinase (T389) 1A5 Monoclonal Antibody (Cell Signalling Technology, Catalogue No. 9206B) and AlphaScreen Streptavidin donor and Protein A acceptor beads (200 ng; Perkin Elmer, Catalogue No. 6760002B and 6760137R respectively) were added and the assay plates were left for about 20 hours at room temperature in the dark. The resultant signals arising from laser light excitation at 680 nm were read using a Packard Envision instrument.

Phosphorylated biotinylated peptide is formed in situ as a result of mTOR mediated phosphorylation. The phosphorylated biotinylated peptide that is associated with AlphaScreen Streptavidin donor beads forms a complex with the p70 S6 Kinase (T389) 1A5 Monoclonal Antibody that is associated with Alphascreen Protein A acceptor beads. Upon laser light excitation at 680 nm, the donor bead: acceptor bead complex produces a signal that can be measured. Accordingly, the presence of mTOR kinase activity results in an assay signal. In the presence of an mTOR kinase inhibitor, signal strength is reduced.

mTOR enzyme inhibition for a given test compound was expressed as an IC₅₀ value.

Enzyme—mTOR Kinase Assay (Echo)

The assay used AlphaScreen technology (Gray et al., Analytical Biochemistry, 2003, 313: 234-245) to determine the ability of test compounds to inhibit phosphorylation by recombinant mTOR.

A C-terminal truncation of mTOR encompassing amino acid residues 1362 to 2549 of mTOR (EMBL Accession No. L34075) was stably expressed as a FLAG-tagged fusion in HEK293 cells as described by Vilella-Bach et al., Journal of Biochemistry, 1999, 274, 4266-4272. The HEK293 FLAG-tagged mTOR (1362-2549) stable cell line was routinely maintained at 37° C. with 5% CO₂ up to a confluency of 70-90% in Dulbecco's modified Eagle's growth medium (DMEM; Invitrogen Limited, Paisley, UK Catalogue No. 41966-029) containing 10% heat-inactivated foetal calf serum (FCS; Sigma, Poole, Dorset, UK, Catalogue No. F0392), 1% L-glutamine (Gibco, Catalogue No. 25030-024) and 2 mg/ml Geneticin (G418 sulfate; Invitrogen Limited, UK Catalogue No. 10131-027). Following expression in the mammalian HEK293 cell line, expressed protein was purified using the FLAG epitope tag using standard purification techniques.

Test compounds were prepared as 10 mM stock solutions in DMSO and diluted in into waterDMSO as required to give a range of final assay concentrations. Aliquots (120 nl 2 μl) of each compound dilution were acoustically dispensed using a Labcyte Echo 550 into a well of a Greiner 384-well low volume (LV) white polystyrene plate (Greiner Bio-one). A 1230 μl mixture of recombinant purified mTOR enzyme, 1 μM biotinylated peptide substrate (Biotin-Ahx-Lys-Lys-Ala-Asn-Gln-Val-Phe-Leu-Gly-Phe-Thr-Tyr-Val-Ala-Pro-Ser-Val-Leu-Glu-Ser-Val-Lys-Glu-NH₂; Bachem UK Ltd), ATP (20 μM) and a buffer solution [comprising Tris-HCl pH7.4 buffer (50 mM), EGTA (0.1 mM), bovine serum albumin (0.5 mg/mL), DTT (1.25 mM) and manganese chloride (10 mM)] was incubated at room temperature for 20 minutes.

Control wells that produced a maximum signal corresponding to maximum enzyme activity were created by using 1005% DMSO instead of test compound. Control wells that produced a minimum signal corresponding to fully inhibited enzyme were created by adding LY294002EDTA (100 uL 83 mM) compound. These assay solutions were incubated for 2 hours at room temperature.

Each reaction was stopped by the addition of 10 μl of a mixture of EDTA (50 mM), bovine serum albumin (BSA; 0.5 mg/mL) and Tris-HCl pH7.4 buffer (50 mM) containing p70 S6 Kinase (T389) 1A5 Monoclonal Antibody (Cell Signalling Technology, Catalogue No. 9206B) and AlphaScreen Streptavidin donor and Protein A acceptor beads (200 ng; Perkin Elmer, Catalogue No. 6760002B and 6760137R respectively) were added and the assay plates were left overnight at room temperature in the dark. The resultant signals arising from laser light excitation at 680 nm were read using a Packard Envision instrument.

Phosphorylated biotinylated peptide is formed in situ as a result of mTOR mediated phosphorylation. The phosphorylated biotinylated peptide that is associated with AlphaScreen Streptavidin donor beads forms a complex with the p70 S6 Kinase (T389) 1A5 Monoclonal Antibody that is associated with Alphascreen Protein A acceptor beads. Upon laser light excitation at 680 nm, the donor bead: acceptor bead complex produces a signal that can be measured. Accordingly, the presence of mTOR kinase activity results in an assay signal. In the presence of an mTOR kinase inhibitor, signal strength is reduced.

mTOR enzyme inhibition for a given test compound was expressed as an IC₅₀ value.

Cellular—Phospho-Ser473 Akt Assay

This assay determines the ability of test compounds to inhibit phosphorylation of Serine 473 in Akt as assessed using Acumen Explorer technology (Acumen Bioscience Limited), a plate reader that can be used to rapidly quantitate features of images generated by laser-scanning.

A MDA-MB-468 human breast adenocarcinoma cell line (LGC Promochem, Teddington, Middlesex, UK, Catalogue No. HTB-132) was routinely maintained at 37° C. with 5% CO₂ up to a confluency of 70-90% in DMEM containing 10% heat-inactivated FCS and 1% L-glutamine.

For the assay, the cells were detached from the culture flask using ‘Accutase’ (Innovative Cell Technologies Inc., San Diego, Calif., USA; Catalogue No. AT104) using standard tissue culture methods and resuspended in media to give 1.7×10⁵ cells per ml. Aliquots (90 μl) were seeded into each of the inner 60 wells of a black Packard 96 well plate (PerkinElmer, Boston, Mass., USA; Catalogue No. 6005182) to give a density of ˜15000 cells per well. Aliquots (90 μl) of culture media were placed in the outer wells to prevent edge effects. The cells were incubated overnight at 37° C. with 5% CO₂ to allow them to adhere.

On day 2, the cells were treated with test compounds and incubated for 2 hours at 37° C. with 5% CO₂. Test compounds were prepared as 10 mM stock solutions in DMSO and serially diluted as required with growth media to give a range of concentrations that were 10-fold the required final test concentrations. Aliquots (10 μl) of each compound dilution were placed in a well (in triplicate) to give the final required concentrations. As a minimum reponse control, each plate contained wells having a final concentration of 100 μM LY294002 (Calbiochem, Beeston, UK, Catalogue No. 440202). As a maximum response control, wells contained 1% DMSO instead of test compound. Following incubation, the contents of the plates were fixed by treatment with a 1.6% aqueous formaldehyde solution (Sigma, Poole, Dorset, UK, Catalogue No. F1635) at room temperature for 1 hour.

All subsequent aspiration and wash steps were carried out using a Tecan 96 well plate washer (aspiration speed 10 mm/sec). The fixing solution was removed and the contents of the plates were washed with phosphate-buffered saline (PBS; 504 Gibco, Catalogue No. 10010015). The contents of the plates were treated for 10 minutes at room temperature with an aliquot (50 μl) of a cell permeabilisation buffer consisting of a mixture of PBS and 0.5% Tween-20. The ‘permeabilisation’ buffer was removed and non-specific binding sites were blocked by treatment for 1 hour at room temperature of an aliquot (50 μl) of a blocking buffer consisting of 5% dried skimmed milk [‘Marvel’ (registered trade mark); Premier Beverages, Stafford, GB] in a mixture of PBS and 0.05% Tween-20. The ‘blocking’ buffer was removed and the cells were incubated for 1 hour at room temperature with rabbit anti phospho-Akt (Ser473) antibody solution (50 μl per well; Cell Signalling, Hitchin, Herts, U.K., Catalogue No 9277) that had been diluted 1:500 in ‘blocking’ buffer. Cells were washed three times in a mixture of PBS and 0.05% Tween-20. Subsequently, cells were incubated for 1 hour at room temperature with Alexafluor488 labelled goat anti-rabbit IgG (50 μl per well; Molecular Probes, Invitrogen Limited, Paisley, UK, Catalogue No. A11008) that had been diluted 1:500 in ‘blocking’ buffer. Cells were washed 3 times with a mixture of PBS and 0.05% Tween-20. An aliquot of PBS (50 μl) was added to each well and the plates were sealed with black plate sealers and the fluorescence signal was detected and analysed.

Fluorescence dose response data obtained with each compound were analysed and the degree of inhibition of Serine 473 in Akt was expressed as an IC₅₀ value.

Compounds that show reduced activity against mTOR may ameliorate off target effects.

Although the pharmacological properties of the compounds of formula (I) vary with structural change as expected, in general, it is believed that activity possessed by compounds of formula (I) may be demonstrated at the following concentrations or doses in one or more of the above tests (a) to (d): —
Test (a): —IC₅₀ versus ATR kinase at less than 10 μM, in particular 0.001-1 μM for many compounds.

The following examples were tested in enzyme assay Test (a) and Enzyme—mTOR Kinase Assay (Echo):

| | | ATR | Enzyme - | mTOR |
|---|---|---|---|---|
| | | number of | mTOR Kinase | number of |
| | ATR average | individual | Assay (Echo) | individual |
| Example | IC50 uM | tests | IC50 uM | tests |
| 1.01 | 0.008093 | 6 | 0.1792 | 4 |
| 1.02 | 0.007666 | 3 | 0.1099 | 2 |
| 1.03 | 0.0275 | 3 | 0.1657 | 2 |
| 1.04 | 0.02264 | 4 | 0.05516 | 2 |
| 1.05 | 0.06586 | 5 | 0.326 | 2 |
| 1.06 | 0.01579 | 2 | 0.07119 | 2 |
| 1.07 | 0.01507 | 2 | | 1 |
| 1.08 | 0.009438 | 4 | 0.2307 | 3 |
| 1.09 | 0.003736 | 5 | | 2 |
| 1.10 | 0.015 | 4 | 0.09575 | 2 |
| 1.11 | 0.01319 | 2 | 0.1222 | 2 |
| 1.12 | 0.03214 | 2 | >90 | 2 |
| 1.13 | 0.05826 | 5 | 1.235 | 2 |
| 1.14 | 0.02239 | 2 | 1.461 | 2 |
| 2.01 | 0.02676 | 2 | 0.4969 | 2 |
| 2.02 | 0.3758 | 2 | 0.4665 | 2 |
| 2.03 | 0.006209 | 3 | 0.09897 | 2 |
| 2.04 | 0.004554 | 2 | 0.2997 | 2 |
| 2.05 | 0.0343 | 2 | 0.04365 | 2 |
| 3.01 | 0.003973 | 5 | 0.02673 | 2 |
| 3.02 | 0.006665 | 2 | 0.09715 | 2 |
| 3.03 | 0.005456 | 2 | 0.04628 | 2 |
| 3.04 | 0.03553 | 2 | 0.2072 | 2 |
| 3.05 | 0.004229 | 3 | 0.05464 | 2 |
| 3.06 | 0.003446 | 2 | 0.03798 | 2 |
| 3.07 | 0.01238 | 2 | 0.02406 | 2 |
| 3.08 | 0.002316 | 3 | 0.2253 | 2 |
| 3.09 | 0.001582 | 2 | 0.4512 | 2 |
| 3.10 | 0.003584 | 3 | 0.1451 | 2 |
| 3.11 | 0.001576 | 2 | 0.07795 | 2 |
| 3.12 | 0.002503 | 2 | 0.4202 | 2 |
| 3.13 | 0.001532 | 2 | 0.2497 | 2 |
| 3.14 | 0.01424 | 2 | 0.692 | 2 |
| 3.15 | 0.002546 | 2 | 0.9803 | 2 |
| 3.16 | 0.00199 | 2 | 0.08584 | 2 |
| 3.17 | 0.002259 | 3 | 0.09777 | 2 |
| 3.18 | 0.007625 | 2 | 0.3832 | 2 |
| 3.19 | 0.09758 | 2 | 2.824 | 2 |
| 3.20 | 0.002501 | 2 | 0.07108 | 2 |
| 3.21 | 0.006891 | 2 | 0.6864 | 2 |
| 3.22 | 0.008188 | 2 | 0.5559 | 2 |
| 3.23 | 0.002466 | 2 | 0.0891 | 1 |
| 3.24 | 0.2612 | 2 | 6.151 | 1 |
| 3.25 | 0.02061 | 2 | 4.236 | 1 |
| 4.01 | 0.02017 | 2 | | |
| 5.01 | 0.006536 | 2 | 0.02097 | 2 |

The following example was tested in the Cellular SRB assay Test (d)

| | | Number of | | | | |
|---|---|---|---|---|---|---|
| | | individual | IC50 | | | |
| Cell | Treatment | tests | ug/ml | S.D. | PF50 | S.D. |
| HT29 | Carboplatin | 2 | 3.97 | 1.34 | | |
| HT29 | Carboplatin + | 2 | 0.39 | 0.12 | 10.08 | 0.22 |
| | 0.3 uM | | | | | |
| | Example 3.01 | | | | | |
| HT29 | Carboplatin + | 2 | 1.96 | 0.50 | 2.02 | 0.16 |
| | 0.1 uM | | | | | |
| | Example 3.01 | | | | | |

Note:
averages are geometric means.

Compounds may be further selected on the basis of further biological or physical properties which may be measured by techniques known in the art and which may be used in the assessment or selection of compounds for therapeutic or prophylactic application.

The compounds of the present invention are advantageous in that they possess pharmacological activity. In particular, the compounds of the present invention modulate ATR kinase. The inhibitory properties of compounds of formula (I) may be demonstrated using the test procedures set out herein and in the experimental section. Accordingly, the compounds of formula (I) may be used in the treatment (therapeutic or prophylactic) of conditions/diseases in human and non-human animals which are mediated by ATR kinase.

The invention also provides a pharmaceutical composition comprising a compound of formula (I), or a pharmaceutically acceptable salt thereof, as defined herein in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 1 mg to 1 g of active agent (more suitably from 1 to 250 mg, for example from 1 to 100 mg) compounded with an appropriate and convenient amount of excipients which may vary from about 5 to about 98 percent by weight of the total composition.

The size of the dose for therapeutic or prophylactic purposes of a compound of formula I will naturally vary according to the nature and severity of the disease state, the age and sex of the animal or patient and the route of administration, according to well known principles of medicine.

In using a compound of formula (I) for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Similarly, for administration by inhalation, a dose in the range, for example, 1 mg/kg to 25 mg/kg body weight will be used. Typically, unit dosage forms will contain about 10 mg to 0.5 g of a compound of this invention.

As stated herein, it is known that ATR kinase have roles in tumourigenesis as well as numerous other diseases. We have found that the compounds of formula (I) possess potent anti-tumour activity which it is believed is obtained by way of inhibition of ATR kinase.

Accordingly, the compounds of the present invention are of value as anti-tumour agents. Particularly, the compounds of the present invention are of value as anti-proliferative, apoptotic and/or anti-invasive agents in the containment and/or treatment of solid and/or liquid tumour disease. Particularly, the compounds of the present invention are expected to be useful in the prevention or treatment of those tumours which are sensitive to inhibition of ATR. Further, the compounds of the present invention are expected to be useful in the prevention or treatment of those tumours which are mediated alone or in part by ATR. The compounds may thus be used to produce an ATR enzyme inhibitory effect in a warm-blooded animal in need of such treatment.

As stated herein, inhibitors of ATR kinase should be of therapeutic value for the treatment of proliferative disease such as cancer and in particular solid tumours such as carcinoma and sarcomas and the leukaemias and lymphoid malignancies and in particular for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias [including acute lymphoctic leukaemia (ALL) and chronic myelogenous leukaemia (CML)], multiple myeloma and lymphomas.

Anti-cancer effects which are accordingly useful in the treatment of cancer in a patient include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. Anti-cancer effects include prophylactic treatment as well as treatment of existing disease.

A ATR kinase inhibitor, or a pharmaceutically acceptable salt thereof, may also be useful for the treatment patients with cancers, including, but not limited to, haematologic malignancies such as leukaemia, multiple myeloma, lymphomas such as Hodgkin's disease, non-Hodgkin's lymphomas (including mantle cell lymphoma), and myelodysplastic syndromes, and also solid tumours and their metastases such as breast cancer, lung cancer (non-small cell lung cancer (NSCL), small cell lung cancer (SCLC), squamous cell carcinoma), endometrial cancer, tumours of the central nervous system such as gliomas, dysembryoplastic neuroepithelial tumour, glioblastoma multiforme, mixed gliomas, medulloblastoma, retinoblastoma, neuroblastoma, germinoma and teratoma, cancers of the gastrointestinal tract such as gastric cancer, oesophagal cancer, hepatocellular (liver) carcinoma, cholangiocarcinomas, colon and rectal carcinomas, cancers of the small intestine, pancreatic cancers, cancers of the skin such as melanomas (in particular metastatic melanoma), thyroid cancers, cancers of the head and neck and cancers of the salivary glands, prostate, testis, ovary, cervix, uterus, vulva, bladder, kidney (including renal cell carcinoma, clear cell and renal oncocytoma), squamous cell carcinomas, sarcomas such as osteosarcoma, chondrosarcoma, leiomyosarcoma, soft tissue sarcoma, Ewing's sarcoma, gastrointestinal stromal tumour (GIST), Kaposi's sarcoma, and paediatric cancers such as rhabdomyosarcomas and neuroblastomas.

The compounds of the present invention and the methods of treatment comprising the administering or use of a ATR kinase inhibitor, or a pharmaceutically acceptable salt thereof, are expected to be particularly useful for the treatment of patients with lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment of patients with acute myeloid leukaemia.

According to a further aspect of the invention there is provided a compound of formula (I), or a pharmaceutically acceptable salt thereof, as defined herein for use as a medicament in a warm-blooded animal such as man.

According to a further aspect of the invention, there is provided a compound of formula (I), or a pharmaceutically acceptable salt thereof, as defined herein for use in the production of an anti-proliferative effect in a warm-blooded animal such as man.

According to a further aspect of the invention, there is provided a compound of formula (I), or a pharmaceutically acceptable salt thereof, as defined herein for use in the production of an apoptotic effect in a warm-blooded animal such as man.

According to a further feature of the invention there is provided a compound of formula (I), or a pharmaceutically acceptable salt thereof, as defined herein for use in a warm-blooded animal such as man as an anti-invasive agent in the containment and/or treatment of proliferative disease such as cancer.

According to a further aspect of the invention, there is provided the use of a compound of formula (I), or a pharmaceutically acceptable salt thereof, as defined herein for the production of an anti-proliferative effect in a warm-blooded animal such as man.

According to a further aspect of the invention, there is provided the use of a compound of formula (I), or a pharmaceutically acceptable salt thereof, as defined herein for use in treating cancer.

According to a further feature of this aspect of the invention there is provided the use of a compound of formula (I), or a pharmaceutically acceptable salt thereof, as defined herein in the manufacture of a medicament for use in the production of an anti-proliferative effect in a warm-blooded animal such as man.

According to a further aspect of the invention, there is provided the use of a compound of formula (I), or a pharmaceutically acceptable salt thereof, as defined herein for the production of an apoptotic effect in a warm-blooded animal such as man.

According to a further feature of this aspect of the invention there is provided the use of a compound of formula (I), or a pharmaceutically acceptable salt thereof, as defined herein in the manufacture of a medicament for use in the production of an apoptotic effect in a warm-blooded animal such as man.

According to a further feature of the invention there is provided the use of a compound of formula (I), or a pharmaceutically acceptable salt thereof, as defined herein in the manufacture of a medicament for use in a warm-blooded animal such as man as an anti-invasive agent in the containment and/or treatment of proliferative disease such as cancer.

According to a further feature of this aspect of the invention there is provided a method for producing an anti-proliferative effect in a warm-blooded animal, such as man, in need of such treatment which comprises administering to said animal an effective amount of a compound of formula (I), or a pharmaceutically acceptable salt thereof, as defined herein.

According to a further feature of this aspect of the invention there is provided a method for producing an anti-invasive effect by the containment and/or treatment of solid tumour disease in a warm-blooded animal, such as man, in need of such treatment which comprises administering to said animal an effective amount of a compound of formula (I), or a pharmaceutically acceptable salt thereof, as defined herein.

According to a further aspect of the invention there is provided the use of a compound of formula (I), or a pharmaceutically acceptable salt thereof, as defined herein in the manufacture of a medicament for use in the prevention or treatment of proliferative disease such as cancer in a warm-blooded animal such as man.

According to a further feature of this aspect of the invention there is provided a method for the prevention or treatment of proliferative disease such as cancer in a warm-blooded animal, such as man, in need of such treatment which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in the prevention or treatment of those tumours which are sensitive to inhibition of ATR kinase.

According to a further feature of this aspect of the invention there is provided the use of a compound of formula (I), or a pharmaceutically acceptable salt thereof, as defined herein in the manufacture of a medicament for use in the prevention or treatment of those tumours which are sensitive to inhibition of ATR kinase.

According to a further feature of this aspect of the invention there is provided a method for the prevention or treatment of those tumours which are sensitive to inhibition of ATR kinase which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in providing a ATR kinase inhibitory effect.

According to a further feature of this aspect of the invention there is provided the use of a compound of formula (I), or a pharmaceutically acceptable salt thereof, as defined herein in the manufacture of a medicament for use in providing a ATR kinase inhibitory effect.

According to a further aspect of the invention there is also provided a method for providing a ATR kinase inhibitory effect which comprises administering an effective amount of a compound of formula I, or a pharmaceutically acceptable salt thereof, as defined herein.

According to a further feature of the invention there is provided a compound of formula I, or a pharmaceutically acceptable salt thereof, as defined herein for use in the treatment of cancer, inflammatory diseases, obstructive airways diseases, immune diseases or cardiovascular diseases.

According to a further feature of the invention there is provided a compound of formula I, or a pharmaceutically acceptable salt thereof, as defined herein for use in the treatment of solid tumours such as carcinoma and sarcomas and the leukaemias and lymphoid malignancies.

According to a further feature of the invention there is provided a compound of formula I, or a pharmaceutically acceptable salt thereof, as defined herein for use in the treatment of cancer of the breast, colorectum, lung (including small cell lung cancer, non-small cell lung cancer and bronchioalveolar cancer) and prostate.

According to a further feature of the invention there is provided a compound of formula (I), or a pharmaceutically acceptable salt thereof, as defined herein for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided a compound of formula (I), or a pharmaceutically acceptable salt thereof, as defined herein for use in the treatment of cancer of the bile duct, bone, bladder, head and neck, kidney, liver, gastrointestinal tissue, oesophagus, ovary, endometrium, pancreas, skin, testes, thyroid, uterus, cervix and vulva, and of leukaemias (including ALL and CML), multiple myeloma and lymphomas.

According to a further feature of the invention there is provided a compound of formula (I), or a pharmaceutically acceptable salt thereof, as defined herein for use in the treatment of lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment acute myeloid leukaemia.

According to a further feature of the invention there is provided the use of a compound of formula (I), or a pharmaceutically acceptable salt thereof, as defined herein in the manufacture of a medicament for use in the treatment of cancer, inflammatory diseases, obstructive airways diseases, immune diseases or cardiovascular diseases.

According to a further feature of the invention there is provided the use of a compound of formula (I), or a pharmaceutically acceptable salt thereof, as defined herein in the manufacture of a medicament for use in the treatment of solid tumours such as carcinoma and sarcomas and the leukaemias and lymphoid malignancies.

According to a further feature of the invention there is provided the use of a compound of formula (I), or a pharmaceutically acceptable salt thereof, as defined herein in the manufacture of a medicament for use in the treatment of cancer of the breast, colorectum, lung (including small cell lung cancer, non-small cell lung cancer and bronchioalveolar cancer) and prostate.

According to a further feature of the invention there is provided the use of a compound of formula (I), or a pharmaceutically acceptable salt thereof, as defined herein in the manufacture of a medicament for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided the use of a compound of formula (I), or a pharmaceutically acceptable salt thereof, as defined herein in the manufacture of a medicament for use in the treatment of lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment acute myeloid leukaemia.

According to a further feature of the invention there is provided a method for treating cancer, inflammatory diseases, obstructive airways diseases, immune diseases or cardiovascular diseases in a warm blooded animal such as man that is in need of such treatment which comprises administering an effective amount of a compound of formula (I), or a pharmaceutically acceptable salt thereof, as defined herein.

According to a further feature of the invention there is provided a method for treating solid tumours such as carcinoma and sarcomas and the leukaemias and lymphoid malignancies in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the breast, colorectum, lung (including small cell lung cancer, non-small cell lung cancer and bronchioalveolar cancer) and prostate in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas in a warm blooded animal such as man that is in need of such treatment which comprises administering an effective amount of a compound of formula (I), or a pharmaceutically acceptable salt thereof, as defined herein.

According to a further feature of the invention there is provided a method for treating lung cancer, prostate cancer, melanoma, ovarian cancer, breast cancer, endometrial cancer, kidney cancer, gastric cancer, sarcomas, head and neck cancers, tumours of the central nervous system and their metastases, and acute myeloid leukaemia in a warm blooded animal such as man that is in need of such treatment which comprises administering an effective amount of a compound of formula (I), or a pharmaceutically acceptable salt thereof, as defined herein.

As stated herein, the in vivo effects of a compound of formula (I) may be exerted in part by one or more metabolites that are formed within the human or animal body after administration of a compound of formula (I).

The invention further relates to combination therapies wherein a compound of formula (I), or a pharmaceutically acceptable salt thereof, or a pharmaceutical composition or formulation comprising a compound of formula (I) is administered concurrently or sequentially or as a combined preparation with another treatment of use in the control of oncology disease.

In particular, the treatment defined herein may be applied as a sole therapy or may involve, in addition to the compounds of the invention, conventional surgery or radiotherapy or chemotherapy. Accordingly, the compounds of the invention can also be used in combination with existing therapeutic agents for the treatment of cancer.

Suitable agents to be used in combination include: —

(i) antiproliferative/antineoplastic drugs and combinations thereof, as used in medical oncology such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)) and inhibitors of cell signalling through MEK and/or Akt kinases;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angiostatin)];
(vi) vascular damaging agents such as combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense agent;
(viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapeutic approaches, including ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

According to a further aspect of the invention there is provided the use of a compound of formula (I), or a pharmaceutically acceptable salt thereof, in the preparation of a medicament for use as an adjunct in cancer therapy or for potentiating tumour cells for treatment with ionising radiation or chemotherapeutic agents.

According to another aspect of the invention there is provided a compound of formula (I), or a pharmaceutically acceptable salt thereof, in combination with ionising radiation or chemotherapeutic agents for use in the treatment of cancer.

The invention will now be further explained by reference to the following illustrative examples.

Unless stated otherwise, starting materials were commercially available. All solvents and commercial reagents were of laboratory grade and were used as received.

### General Experimental

The invention will now be illustrated in the following Examples in which, generally:

(i) operations were carried out at room temperature (RT), i.e. in the range 17 to 25° C. and under an atmosphere of an inert gas such as N₂ or Ar unless otherwise stated;
(ii) in general, the course of reactions was followed by thin layer chromatography (TLC) and/or analytical high performance liquid chromatography (HPLC) which was usually coupled to a mass spectrometer (LCMS). The reaction times that are given are not necessarily the minimum attainable;
(iii) when necessary, organic solutions were dried over anhydrous MgSO₄ or Na₂SO₄, work-up procedures were carried out using traditional phase separating techniques or by using SCX as described in (xiii), evaporations were carried out either by rotary evaporation in vacuo or in a Genevac HT-4/EZ-2 or Biotage V10;
(iv) yields, where present, are not necessarily the maximum attainable, and when necessary, reactions were repeated if a larger amount of the reaction product was required;
(v) in general, the structures of the end-products of the formula (I) were confirmed by nuclear magnetic resonance (NMR) and/or mass spectral techniques; electrospray mass spectral data were obtained using a Waters ZMD or Waters ZQ LC/mass spectrometer acquiring both positive and negative ion data, and generally, only ions relating to the parent structure are reported; proton NMR chemical shift values were measured on the delta scale using either a Bruker DPX300 spectrometer operating at a field strength of 300 MHz, a Bruker DRX400 operating at 400 MHz, a Bruker DRX500 operating at 500 MHz or a Bruker AV700 operating at 700 MHz. Unless otherwise stated, NMR spectra were obtained at 400 MHz in d⁶-dimethylsulfoxide. The following abbreviations have been used: s, singlet; d, doublet; t, triplet; q, quartet; m, multiplet; br, broad; qn, quintet;
(vi) Unless stated otherwise compounds containing an asymmetric carbon and/or sulphur atom were not resolved;
(vii) Intermediates were not necessarily fully purified but their structures and purity were assessed by TLC, analytical HPLC, and/or NMR analysis and/or mass spectrometry;
(viii) unless otherwise stated, flash column chromatography (FCC) was performed on Merck Kieselgel silica (Art. 9385) or on Silicycle cartridges (40-63 μm silica, 4 to 330 g weight) or on Grace resolv cartridges (4-120 g) either manually or automated using an Isco Combi Flash Companion system;
(ix) Preparative reverse phase HPLC (RP HPLC) was performed on C18 reversed-phase silica, for example on a Waters ‘Xterra’ or ‘XBridge’ preparative reversed-phase column (5 μm silica, 19 mm diameter, 100 mm length) or on a Phenomenex “Gemini” or ‘AXIA’ preparative reversed-phase column (5 μm silica, 110A, 21.1 mm diameter, 100 mm length) using decreasingly polar mixtures as eluent, for example [containing 1-5% formic acid or 1-5% aqueous ammonium hydroxide (d=0.88)] as solvent A and acetonitrile as solvent B or MeOH:MeCN 3:1; a typical procedure would be as follows: a solvent gradient over 9.5 minutes, at 25 mL per minute, from a 85:15 (or alternative ratio as appropriate) mixture of solvents A and B respectively to a 5:95 mixture of solvents A and B;
(x) the following analytical HPLC methods were used; in general, reverse-phase silica was used with a flow rate of about 1 mL/minute and detection was by Electrospray Mass Spectrometry and by UV absorbance at a wavelength of 254 nm. Analytical HPLC was performed on C18 reverse-phase silica, on a Phenomenex “Gemini” preparative reversed-phase column (5 μm silica, 110 A, 2 mm diameter, 50 mm length) using decreasingly polar mixtures as eluent, for example decreasingly polar mixtures of water (containing 0.1% formic acid or 0.1% ammonia) as solvent A and acetonitrile as solvent B or MeOH: MeCN 3:1. A typical analytical HPLC method would be as follows: a solvent gradient over 4 minutes, at approximately 1 mL per minute, from a 95:5 mixture of solvents A and B respectively to a 5:95 mixture of solvents A and B;
(xi) Where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;
(xii) Where reactions refer to the use of a microwave, one of the following microwave reactors were used: Biotage Initiator, Personal Chemistry Emrys Optimizer, Personal Chemistry Smithcreator or CEM Explorer;
(xiii) Compounds were purified by strong cation exchange (SCX) chromatography using Isolute SPE flash SCX-2 column (International Sorbent Technology Limited, Mid Glamorgan, UK);
(xiv) In addition to the ones mentioned above, the following abbreviations have been used:

| | | | |
|---|---|---|---|
| DMF | N,N- | DMA | N,N-dimethylacetamide |
| | dimethylformamide | | |
| DCM | dichloromethane | THF | tetrahydrofuran |
| conc. | concentrated | m/z | mass spectrometry peak(s) |
| TBAF | tetra n-butylammonium | NMP | 1-methylpyrrolidin-2-one |
| | fluoride | | |
| EtOAc | ethyl acetate | DIPEA | N,N-diisopropylethylamine |
| DME | 1,2-dimethoxyethane | MeOH | methanol |
| MeCN | acetonitrile | TBAB | tetra n-butylammonium |
| | | | bromide |
| h | hour(s) | EtOH | ethanol |

### EXAMPLE 1.01

### 4-{4-[1-(Methylsulfonyl)cyclopropyl]-6-morpholin-4-ylpyrimidin-2-yl}-1H-indole

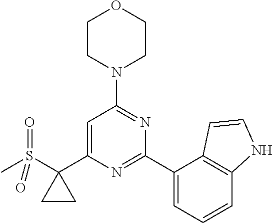

Bis(triphenylphosphine)palladium(II) dichloride (82 mg, 0.12 mmol), 4-(2-chloro-6-(1-(methylsulfonyl)cyclopropyl)pyrimidin-4-yl)morpholine (371 mg, 1.17 mmol), 2M aqueous sodium carbonate (3.50 mL, 7.00 mmol) and 1H-indol-4-ylboronic acid (225 mg, 1.40 mmol) were suspended in 18% DMF in 7:3:2 DME:water:EtOH (8 mL) and sealed into a microwave tube. The mixture was heated to 110° C. for 1 hour in a microwave reactor and then allowed to cool to RT. The mixture was purified by preparative HPLC using decreasingly polar mixtures of water (containing 1% NH3) and MeCN as eluents. Fractions containing the desired compound were combined and then evaporated. The residue was purified by preparative HPLC using decreasingly polar mixtures of water (containing 0.1% formic acid) and MeCN as eluents. Fractions containing the desired compound were combined and then evaporated to afford the title compound (264 mg, 57%); ¹H NMR (400 MHz, DMSO-d₆) 1.60-1.63 (2H, m), 1.71-1.74 (2H, m), 3.10 (3H, s), 3.74-3.78 (8H, m), 6.89 (1H, s), 7.21 (1H, t), 7.31 (1H, d), 7.46 (1H, t), 7.55 (1H, d), 8.04-8.06 (1H, m), 11.25 (1H, s); m/z: (ES+) MH⁺, 399.13.

Alternatively, the title compound can be prepared as follows:

Tetrakis (triphenylphosphine)Pd(0) (56.1 mg, 0.05 mmol) was added to 4-(6-(1-(methylsulfonyl)cyclopropyl)-2-(methylthio)pyrimidin-4-yl)morpholine (200 mg, 0.61 mmol), 1H-indol-4-ylboronic acid (195 mg, 1.21 mmol) and (thiophene-2-carbonyloxy)copper (301 mg, 1.58 mmol) in a mixture of dioxane (10 mL) and DMA (2 mL) under an atmosphere of nitrogen. The resulting mixture was stirred at 90° C. for 18 hours. The mixture was purified by ion exchange chromatography, using an SCX column. The product was eluted from the column using 7M NH3 in MeOH and fractions containing product were combined and evaporated. The residue was purified by preparative HPLC using decreasingly polar mixtures of water (containing 1% NH3) and MeCN as eluents. Fractions containing the desired compound were combined and evaporated to afford the title compound (58 mg, 24%); ¹H NMR (400 MHz, DMSO-d₆) 1.59-1.62 (2H, m), 1.71-1.74 (2H, m), 3.25 (3H, s), 3.76-3.78 (8H, m), 6.89 (1H, s), 7.20 (1H, t), 7.30 (1H, t), 7.45 (1H, t), 7.55 (1H, d), 8.03-8.05 (1H, m), 11.24 (1H, s); m/z: (ES+) MH⁺, 399.15.

The 4-(2-chloro-6-(1-(methylsulfonyl)cyclopropyl)pyrimidin-4-yl)morpholine, used as starting material, can be prepared as follows:

a) 6-(Chloromethyl)-1H-pyrimidine-2,4-dione (175 g, 1.09 mol) was dissolved in DMF (2 L) and then sodium methanesulfinate (133.5 g, 1.31 mol) was added. The mixture was heated to 125° C. for 2 h. After cooling to RT, the mixture was filtered and the filtrate was concentrated in vacuo. The crude material was washed with water, filtered, then triturated with toluene. The solid was filtered then triturated with isohexane to provide 6-(methylsulfonylmethyl)-1H-pyrimidine-2,4-dione (250 g), which was used without further purification.

b) 6-(Methylsulfonylmethyl)-1H-pyrimidine-2,4-dione (132 g, 0.65 mol) was added to POCl₃ (1.2 L) and the mixture was heated to reflux for 16 h. The excess POCl₃ was removed in vacuo, the residue azeotroped with toluene (2×500 mL) and residue was dissolved in DCM. This solution was then poured slowly onto ice (4 L) and the mixture was stirred for 20 minutes. The mixture was then extracted with DCM (3×1 L) (insoluble black material was filtered off and discarded) and EtOAc (2×1 L). The organic extracts were combined, dried and concentrated in vacuo to provide 2,4-dichloro-6-(methylsulfonylmethyl)pyrimidine (51 g), which was used without further purification; ¹H NMR (400 MHz, DMSO-d⁶) 3.13 (s, 3H), 4.79 (s, 2H), 7.87 (s, 1H); m/z: (ESI+) MH⁺, 239.

c) Triethylamine (6.78 mL) was added to a cooled (−5° C.) suspension of 2,4-dichloro-6-(methylsulfonylmethyl)pyrimidine (10.56 g) in DCM (230 mL). A solution of morpholine (3.85 mL) in DCM (30 mL) was then added dropwise while keeping the temperature below −5° C. The mixture was then stirred at RT for 1 h. The solution was then washed with water (300 mL), dried (MgSO₄) and concentrated in vacuo. Purification by FCC eluting with 1:1 EtOAc-DCM provided 2-chloro-4-(methylsulfonylmethyl)-6-morpholin-4-yl-pyrimidine (6.81 g) as a solid; ¹H NMR (400 MHz, DMSO-d⁶) 3.12 (3H, s), 3.63 (4H, s), 3.68-3.70 (4H, m), 4.45 (2H, s), 6.96 (1H, s); m/z: (ESI+) MH⁺, 292.

d) NaOH solution (9.60 mL, 95.97 mmol) was added to a mixture of 2-chloro-4-(methylsulfonylmethyl)-6-morpholin-4-yl-pyrimidine (2.80 g, 9.60 mmol), 1,2-dibromoethane (1.654 mL, 19.19 mmol) and TBAB (0.619 g, 1.92 mmol) in toluene (120 mL). The resulting solution was then heated to 60° C. for 3 h. The mixture was then concentrated in vacuo to provide a residue which was dissolved in EtOAc (200 mL). The solution was washed with water (200 mL) and then saturated brine (100 mL). The solution was then dried (MgSO₄) and concentrated in vacuo. The residue was purified by chromatography on silica, eluting with a gradient of 0-2.5% MeOH in DCM provided 4-(2-chloro-6-(1-(methylsulfonyl)cyclopropyl)pyrimidin-4-yl)morpholine (2.88 g); ¹H NMR (400 MHz, DMSO-d⁶) 1.49-0.51 (2H, m), 1.62-1.65 (2H, m), 3.19 (3H, s), 3.67 (8H, d), 6.96 (1H, s); m/z: (ESI+) MH⁺, 318.

The 4-(6-(1-(methylsulfonyl)cyclopropyl)-2-(methylthio)pyrimidin-4-yl)morpholine, used as starting material, was prepared as follows:

a) Methyl 2-(methylsulfonyl)acetate (7.12 g, 46.80 mmol) was dissolved in DMF (60 mL), to this was added sodium hydride (4.08 g, 85.10 mmol) and the mixture was stirred for 5 minutes before the addition of 4,6-dichloro-2-(methylthio)pyrimidine (8.3 g, 42.55 mmol). The mixture was stirred for 36 hours before the addition of morpholine (18.53 mL, 212.74 mmol), and then the reaction was stirred for 1 hour at 50° C. The reaction was quenched with 2M HCl (100 mL), and then extracted with Et2O (3×100 mL). The organic layer was separated and then dried over MgSO4, filtered and then evaporated. The residue was purified by chromatography on silica eluting with a gradient of 50 to 100% EtOAc in isohexane. Fractions containing product were combined and evaporated. The residue was triturated with Et2O under sonication to afford methyl 2-(methylsulfonyl)-2-(2-(methylthio)-6-morpholinopyrimidin-4-yl)acetate (8.70 g, 57%); ¹H NMR (400 MHz, CDCl₃) 2.46 (3H, s), 3.19 (3H, s), 3.70-3.64 (4H, m), 3.78-3.75 (4H, m), 3.82 (2H, s), 4.84 (1H, s), 6.49 (1H, s); m/z: (ESI+) MH⁺, 362.39.

b) Methyl 2-(methylsulfonyl)-2-(2-(methylthio)-6-morpholinopyrimidin-4-yl)acetate (0.568 g, 1.57 mmol) was dissolved in MeOH (20 mL) and water (5 mL), to this was added NaOH (0.189 g, 4.71 mmol) and the mixture was stirred at 60° C. for 1 hour. The mixture was cooled and then filtered. The residue was washed with MeOH (25 mL) and allowed to dry in air to afford 4-(6-(methylsulfonylmethyl)-2-(methylthio)pyrimidin-4-yl)morpholine (0.460 g, 96%) which was used in the next step without further purification; ¹H NMR (400 MHz, CDCl₃) 2.49 (3H, s), 3.01 (3H, s), 3.67-3.62 (4H, m), 3.78-3.76 (4H, m), 4.12 (2H, s), 6.30 (1H, s); m/z: (ESI+) MH⁺, 304.09.

c) A 50% aqueous solution of NaOH (1.846 mL, 34.61 mmol) was added to 4-(6-(methylsulfonylmethyl)-2-(methylthio)pyrimidin-4-yl)morpholine (210 mg, 0.69 mmol), 1,2-dibromoethane (0.179 mL, 2.08 mmol) and TBAB (22.31 mg, 0.07 mmol) in toluene (25 mL) at RT. The resulting mixture was stirred at 60° C. for 18 hours. Water (30 mL) was added and the mixture was extracted with toluene (50 mL×2). The toluene layers were dried over MgSO4, filtered and then evaporated to afford 4-(6-(1-(methylsulfonyl)cyclopropyl)-2-(methylthio)pyrimidin-4-yl)morpholine (210 mg, 92%); m/z: (ESI+) MH⁺, 330.

### EXAMPLE 1.02

### 6-Methyl-4-{4-[1-(methylsulfonyl)cyclopropyl]-6-morpholin-4-ylpyrimidin-2-yl}-1H-indole

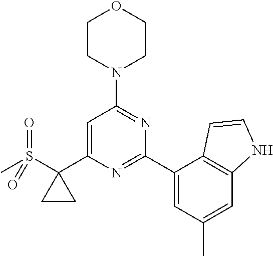

Tris(dibenzylideneacetone)dipalladium(0)-chloroform adduct (4.89 mg, 4.72 μmol) and tricyclohexylphosphine (7.06 mg, 0.03 mmol) were added to 4-bromo-6-methyl-1H-indole (66.1 mg, 0.31 mmol), potassium acetate (46.3 mg, 0.47 mmol) and bis(pinacolato)diboron (88 mg, 0.35 mmol) in dioxane (5 mL) under a nitrogen atmosphere. The resulting suspension was stirred at 90° C. for 18 hours and then 4-(2-chloro-6-(1-(methylsulfonyl)cyclopropyl)pyrimidin-4-yl)morpholine (100 mg, 0.31 mmol), tetrakis(triphenylphosphine)palladium(0) (18.18 mg, 0.02 mmol) and sodium carbonate (2M aqueous solution) (0.629 mL, 1.26 mmol) were added; the resulting suspension was stirred at 90° C. for 18 hours. The reaction mixture was filtered through Celite and the residue washed with DCM. The liquors were purified by ion exchange chromatography using an SCX column. The desired product was eluted from the column using 20% 7M NH3/MeOH in DCM and fractions containing product were combined and evaporated. The residue was purified by preparative HPLC using decreasingly polar mixtures of water (containing 1% NH3) and MeCN as eluents. Fractions containing the desired compound were combined and evaporated to afford the title compound (1.6 mg, 1%); ¹H NMR (400 MHz, DMSO-d⁶) 1.61 (q, 2H), 1.72 (q, 2H), 2.48 (s, 3H), 3.29 (s, 3H), 3.72-3.80 (m, 8H), 6.89 (s, 1H), 7.23 (s, 1H), 7.35 (d, 2H), 7.87 (s, 1H), 11.09 (s, 1H); m/z: (ESI+) MH⁺, 413.68.

### EXAMPLE 1.03

### 2-Methyl-4-{4-[1-(methylsulfonyl)cyclopropyl]-6-morpholin-4-ylpyrimidin-2-yl}-1H-indole

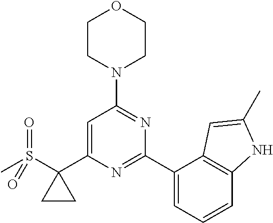

1,1′-Bis(diphenylphosphino)ferrocene-palladium dichloride (12.94 mg, 0.02 mmol) was added to a degassed solution of 2-methyl-1H-indol-4-yl trifluoromethanesulfonate (88 mg, 0.31 mmol), bis(pinacolato)diboron (84 mg, 0.33 mmol), 1,1′-bis(diphenylphosphino)ferrocene (8.82 mg, 0.02 mmol) and potassium acetate (93 mg, 0.94 mmol) in dioxane (5 mL) under a nitrogen atmosphere and the resulting mixture was stirred at 90° C. for 24 hours. 4-(2-Chloro-6-(1-(methylsulfonyl)cyclopropyl)pyrimidin-4-yl)morpholine (100 mg, 0.31 mmol), tetrakis(triphenylphosphine)palladium(0) (18.18 mg, 0.02 mmol) and sodium carbonate (2M aqueous solution) (0.084 mL, 2 mmol) were added and the mixture heated at 90° C. for 24 hours. The reaction mixture was filtered through Celite and the residue washed with DCM. The filtrate was purified by ion exchange chromatography, using an SCX column and the desired product was eluted from the column using 20% 7M NH3/MeOH in DCM. Fractions containing product were combined and evaporated. The residue was purified by preparative HPLC using decreasingly polar mixtures of water (containing 1% NH3) and MeCN as eluents. Fractions containing product were combined and evaporated to afford the title compound (14 mg, 11%); ¹H NMR (700 MHz, DMSO-d⁶) 1.58 (q, 2H), 1.70 (q, 2H), 2.42 (s, 3H), 3.24 (s, 3H), 3.70-3.75 (m, 8H), 6.85 (s, 1H), 6.98 (t, 1H), 7.07 (t, 1H), 7.38 (d, 1H), 7.97 (d, 1H), 11.03 (s, 1H); m/z: (ESI+) MH⁺, 413.63.

The 2-methyl-1H-indol-4-yl trifluoromethanesulfonate, used as starting material, was prepared as follows:

2,6-Lutidine (0.036 mL, 0.31 mmol) and trifluoromethanesulfonic anhydride (0.063 mL, 0.37 mmol) were added to 2-methyl-1H-indol-4-ol (0.046 g, 0.31 mmol) in DCM (5 mL) cooled to 0° C. The resulting solution was stirred for 1 hour and the reaction mixture was then diluted with water (5 mL). The mixture was separated and the organic layer dried over MgSO4, filtered and then evaporated to afford 2-methyl-1H-indol-4-yl trifluoromethanesulfonate which was used directly in the next step without purification; m/z: (ESI−) M-H⁻, 279.

### EXAMPLE 1.04

### 6-Methoxy-4-{4-[1-(methylsulfonyl)cyclopropyl]-6-morpholin-4-ylpyrimidin-2-yl}-1H-indole

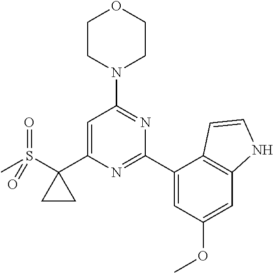

1,1′-Bis(diphenylphosphino)ferrocenedichloropalladium(II) (49 mg, 0.06 mmol) was added to 4-bromo-6-methoxy-1H-indole (0.124 g, 0.55 mmol), potassium acetate (0.137 g, 1.40 mmol) and bis(pinacolato)diboron (0.254 g, 1.00 mmol) in dioxane (5 mL) and the resulting mixture was stirred at 100° C. for 3 hours. 4-(2-Chloro-6-(1-(methylsulfonyl)cyclopropyl)pyrimidin-4-yl)morpholine (0.127 g, 0.4 mmol), tetrakis (triphenylphosphine)Pd(0) (0.046 g, 0.04 mmol) and sodium carbonate (2M solution) (0.800 mL, 1.60 mmol) were added and the resulting mixture was stirred at 100° C. for 16 hours. The reaction mixture was filtered through Celite and the residue washed with DCM; the liquors were purified by ion exchange chromatography, using an SCX column. The desired product was eluted from the column using 50% 7M NH3/MeOH in DCM and fractions containing product were combined and evaporated. The residue was purified by preparative HPLC using decreasingly polar mixtures of water (containing 1% NH3) and MeCN as eluents. Fractions containing the desired compound were combined and evaporated. The residue was purified by ion exchange chromatography, using an SCX column. The desired product was eluted from the column using 50% 7M NH3/MeOH in DCM and fractions containing product were combined and evaporated to afford the title compound (32 mg, 15%); ¹H NMR (400 MHz, DMSO-d⁶) 1.61 (dd, 2H), 1.72 (dd, 2H), 3.29 (s, 3H), 3.73-3.79 (m, 8H), 3.83 (s, 3H), 6.90 (s, 1H), 7.08 (d, 1H), 7.20 (t, 1H), 7.31 (t, 1H), 7.69 (d, 1H), 11.04 (s, 1H); m/z: (ESI+) MH⁺, 429.3.

The following compounds were prepared in a similar way to the method described for Example 1.04, using 4-(2-chloro-6-(1-(methylsulfonyl)cyclopropyl)pyrimidin-4-yl)morpholine and the appropriate substituted 4-bromoindole.

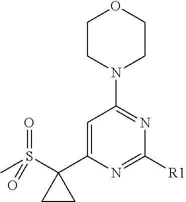

| | | | | m/z |
|---|---|---|---|---|
| Ex. | | | | (ESI+) |
| No. | R1 | Name | ¹H NMR | MH⁺ |
| 1.05 | 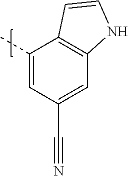 | 4-{4-[1- (methylsulfonyl)cyclopropyl]- 6-morpholin-4-ylpyrimidin-2- yl}-1H-indole-6-carbonitrile | 1.63 (dd, 2H), 1.74 (dd, 2H), 3.26 (s, 3H), 3.71- 3.83 (m, 8H), 6.98 (s, 1H), 7.45 (d, 1H), 7.79 (d, 1H), 8.04 (d, 1H), 8.28 (d, 1H), 11.86 (s, 1H) | 424.29 |
| 1.06 | 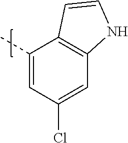 | 6-chloro-4-{4-[1- (methylsulfonyl)cyclopropyl]- 6-morpholin-4-ylpyrimidin-2- yl}-1H-indole | 1.61 (dd, 2H), 1.73 (dd, 2H), 3.27 (s, 3H), 3.71- 3.81 (m, 8H), 6.95 (s, 1H), 7.32 (s, 1H), 7.52 (t, 1H), 7.59 (dd, 1H), 7.99 (d, 1H), 11.40 (s, 1H) | 433.3 |
| 1.07 | 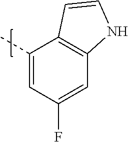 | 6-fluoro-4-{4-[1- (methylsulfonyl)cyclopropyl]- 6-morpholin-4-ylpyrimidin-2- yl}-1H-indole | 1.62 (dd, 2H), 1.73 (dd, 2H), 3.27 (s, 3H), 3.72- 3.81 (m, 8H), 6.94 (s, 1H), 7.30-7.36 (m, 2H), 7.47 (t, 1H), 7.82 (dd, 1H), 11.32 (s, 1H) | 417.28 |

### EXAMPLE 1.08

### 4-{4-[4-(Methylsulfonyl)piperidin-4-yl]-6-morpholin-4-ylpyrimidin-2-yl}-1H-indole

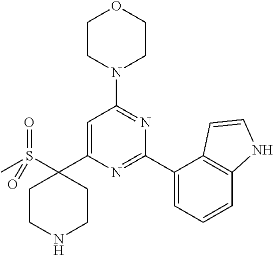

Dichlorobis(triphenylphosphine)palladium(11) (0.016 g, 0.02 mmol) was added in one portion to tert-butyl 4-(2-chloro-6-morpholinopyrimidin-4-yl)-4-(methylsulfonyl)piperidine-1-carboxylate (1.075 g, 2.33 mmol), 2M aqueous sodium carbonate solution (1.399 mL, 2.80 mmol) and 1H-indol-4-ylboronic acid (0.413 g, 2.57 mmol) in DME:water 4:1 (24 mL) at 22° C. The mixture was sealed into four separate microwave tubes which were then heated to 110° C. for 1 hour in a microwave reactor and then allowed to cool to RT. The separate reaction mixtures were combined and then filtered; the resultant solution was treated with 4M HCl in dioxane (4 mL) and stirred overnight at RT. The crude product was purified by ion exchange chromatography, using an SCX column. The desired product was eluted from the column using 7M NH3/MeOH and fractions containing product were combined and evaporated. The residue was purified by preparative HPLC using decreasingly polar mixtures of water (containing 1% NH3) and 3:1 MeOH:MeCN as eluents. Fractions containing product were combined and evaporated to afford the title compound (0.270 g, 26%); ¹H NMR (400 MHz, DMSO-d⁶) 2.03-2.11 (2H, m), 2.41 (2H, t), 2.82 (3H, s), 2.86 (2H, s), 2.97 (2H, d), 3.78 (8H, s), 6.93 (1H, s), 7.20 (1H, t), 7.29 (1H, d), 7.46-7.47 (1H, m), 7.55 (1H, d), 8.09-8.11 (1H, m), 11.29 (1H, s); m/z: (ESI+) MH⁺, 442.15.

The tert-butyl 4-(2-chloro-6-morpholinopyrimidin-4-yl)-4-(methylsulfonyl)piperidine-1-carboxylate, used as starting material, was prepared as follows:

a) A solution of 4-(2-chloro-6-(methylsulfonylmethyl)pyrimidin-4-yl)morpholine (3 g, 10.28 mmol) in NMP (23 mL) was treated with sodium hydride (1.357 g, 33.93 mmol). The mixture was stirred at RT for 10 minutes before being treated with tetrabutylammonium bromide (4.97 g, 15.42 mmol) and N-benzyl-2-chloro-N-(2-chloroethyl)ethanamine hydrochloride (3.04 g, 11.31 mmol). The reaction mixture was stirred for 5 minutes at RT and then heated to 50° C. for 1 hour and then to 80° C. for 1.5 hours. The mixture was allowed to cool to RT and then quenched by the addition of aqueous saturated ammonium chloride. The mixture was extracted with EtOAc and the organic solution was washed three times with water, dried over MgSO4, filtered, and concentrated under reduced pressure. The residue was purified by chromotography on silica using a gradient elution of 10% EtOAc/DCM to 70% EtOAc/DCM to afford 4-(6-(1-benzyl-4-(methylsulfonyl)piperidin-4-yl)-2-chloropyrimidin-4-yl)morpholine (2.000 g, 43%); m/z: (ESI+) MH⁺, 451.03.

b) 1-Chloroethyl carbonochloridate (0.957 mL, 8.87 mmol) was added dropwise to a solution of 4-(6-(benzyl-4-(methylsulfonyl)piperidin-4-yl)-2-chloropyrimidin-4-yl)morpholine in DCM (10 mL) at RT and the solution was then heated at reflux for 3 hours. The solution was allowed to cool and then diluted with MeOH (10 mL) and stirred overnight. The mixture was treated with di-tert-butyl dicarbonate (2.129 g, 9.76 mmol) and N-ethyl-N-isopropylpropan-2-amine (1.545 mL, 8.87 mmol) and the solution allowed to stir for 2 hours. The reaction mixture was diluted with DCM (20 mL) and then washed with water (50 mL) and saturated brine (50 mL). The organic layer was dried over MgSO4, filtered and evaporated. The residue was purified by chromatography on silica with an elution gradient of 10 to 50% EtOAc in DCM. Fractions containing product were combined and evaporated to afford tert-butyl 4-(2-chloro-6-morpholinopyrimidin-4-yl)-4-(methylsulfonyl)piperidine-1-carboxylate (1.075 g, 53%); m/z: (ESI+) M-H, 459.45.

The following compounds were prepared in a similar way to the method described for Example 1.08, using the appropriate 2-chloropyrimidine starting material and 1H-indol-4-ylboronic acid.

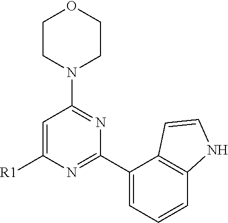

| | | | | m/z |
|---|---|---|---|---|
| Ex. | | | | (ESI+) |
| No. | R1 | Name | ¹H NMR | MH⁺ |
| 1.09^{a} | 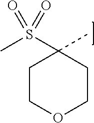 | 4-{4-[4- (Methylsulfonyl)tetrahydro-2H- pyran-4-yl]-6-morpholin-4- ylpyrimidin-2-yl}-1H-indole | 2.21-2.27 (2H, m), 2.85 (3H, s), 2.87-2.90 (2H, m), 3.24-3.28 (2H, m), 3.72- 3.80 (8H, m), 3.92 3.97 (2H, m), 6.98 (1H, s), 7.20 (1H, t), 7.26-7.27 (1H, m), 7.45- 7.46 (1H, m), 7.55 (1H, d), 8.09 (1H, d) | 443.14 |
| 1.10^{b} | 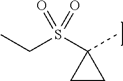 | 4-{4-[1- (ethylsulfonyl)cyclopropyl]-6- morpholin-4-ylpyrimidin-2-yl}- 1H-indole | 1.30 (3H, t), 1.60- 1.61 (2H, m), 1.68- 1.69 (2H, m), 3.45 (2H, q), 3.74- 3.76 (8H, m), 6.90 (1H, s), 7.21 (1H, t), 7.30-7.31 (1H, m), 7.45-7.46 (1H, m), 7.55 (1H, d), 8.04 (1H, d), 11.23 (1H, s) | 413.12 |
| 1.11^{c} | 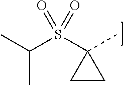 | 4-(4-{1-[(1- methylethyl)sulfonyl]cyclopropyl}- 6-morpholin-4-ylpyrimidin-2-yl)- 1H-indole | 1.32 (6H, d), 1.63- 1.67 (4H, m), 3.74- 3.76 (8H, m), 6.91 (1H, s), 7.21 (1H, t), 7.33-7.34 (1H, m), 7.46-7.47 (1H, m), 7.55 (1H, d), 8.06 (1H, d), 11.23 (1H, s) | 427.12 |
| 1.12^{d} | 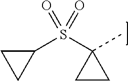 | 4-{4-[1- (cyclopropylsulfonyl)cyclopropyl]- 6-morpholin-4-ylpyrimidin-2-yl}- 1H-indole | 0.95-1.00 (4H, m), 1.63-1.63 (2H, m), 1.70- 1.72 (2H, m), 3.03- 3.08 (1H, m), 3.76 (8H, s), 6.96 (1H, s), 7.20 (1H, t), 7.35-7.37 (1H, m), 7.45-7.47 (1H, m), 7.54 (1H, d), 8.07-8.10 (1H, m), 11.23 (1H, s) | 425.15 |
| 1.13^{e} | 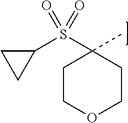 | 4-{4-[4- (cyclopropylsulfonyl)tetrahydro- 2H-pyran-4-yl]-6-morpholin-4- ylpyrimidin-2-yl}-1H-indole | 0.78-0.81 (2H, m), 0.82-0.88 (2H, m), 2.23- 2.31 (2H, m), 2.96- 3.00 (2H, m), 3.74-3.79 (8H, m), 3.92-3.97 (2H, m), 7.00 (1H, s), 7.20 (1H, t), 7.31 (1H, s), 7.44- 7.46 (1H, m), 7.55 (1H, d), 8.12 (1H, d), 11.24 (1H, s) | 469.16 |
| 1.14^{f} | 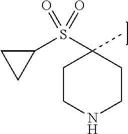 | 4-{4-[4- (cyclopropylsulfonyl)piperidin-4- yl]-6-morpholin-4-ylpyrimidin-2- yl}-1H-indole | 0.75-0.77 (2H, m), 0.93-0.95 (2H, m), 2.11- 2.19 (2H, m), 2.43- 2.51 (2H, m), 2.87-2.93 (4H, m), 3.22-3.25 (1H, m), 3.76- 3.78 (8H, m), 6.90 (1H, s), 7.17 (1H, t), 7.30-7.34 (1H, m), 7.43-7.45 (1H, m), 7.53 (1H, d), 8.11 (1H, d), 10.98 (1H, s) | 468.19 |

^{a}The 4-(2-chloro-6-(4-(methylsulfonyl)tetrahydro-2H-pyran-4-yl)pyrimidin-4-yl)morpholine, used as starting material, was prepared as follows:

A 50% aqueous NaOH solution (6.67 mL) was added to 4-(2-chloro-6-(methylsulfonylmethyl)pyrimidin-4-yl)morpholine (880 mg, 3.02 mmol), tetrabutylammonium bromide (97 mg, 0.30 mmol) and 1-bromo-2-(2-bromoethoxy)ethane (2099 mg, 9.05 mmol) in DCM (20 mL) at 22° C. The resulting mixture was stirred at 20° C. for 6 hours. The mixture was diluted with water (50 mL) and the phases separated. The organic phase was washed twice with water (25 mL) and then dried over MgSO4, filtered and evaporated. The residue was purified by chromatography on silica with an elution gradient of 0 to 40% EtOAc in DCM. Fractions containing product were combined and evaporated to afford 4-(2-chloro-6-(4-(methylsulfonyl)tetrahydro-2H-pyran-4-yl)pyrimidin-4-yl)morpholine (698 mg, 64%); ¹H NMR (400 MHz, DMSO-d⁶) 2.00-2.14 (2H, m), 2.44-2.47 (2H, m), 2.64-2.67 (2H, m), 2.85 (3H, s), 3.15-3.18 (2H, m), 3.65-3.72 (8H, m), 3.89-3.92 (2H, m), 7.02 (1H, s); m/z: (ESI+) MH⁺, 362.04.

^{b}The 4-(2-chloro-6-(1-(ethylsulfonyl)cyclopropyl)pyrimidin-4-yl)morpholine, used as starting material, was prepared as follows:

a) Sodium ethanesulfinate (1.026 g, 8.83 mmol) was added in one portion to 4-(2-chloro-6-(iodomethyl)pyrimidin-4-yl)morpholine (3 g, 8.83 mmol) in DMF (58.9 mL). The resulting mixture was stirred at 25° C. for 18 hours. The reaction mixture was diluted with DCM and washed with water (2×300 mL), aqueous sodium thiosulphate (400 mL) and then brine (400 mL). The organic phase was dried over MgSO4 and then concentrated in vacuo. The residue was purified by chromatography on silica with an elution gradient of 0 to 50% EtOAc in DCM. Fractions containing product were combined and evaporated to afford 4-(2-chloro-6-(ethylsulfonylmethyl)pyrimidin-4-yl)morpholine (1.310 g, 49%); ¹H NMR (400 MHz, DMSO-d⁶) 1.28 (3H, t), 3.25 (2H, q), 3.63-3.71 (8H, m), 4.43 (2H, s), 6.96 (1H, s); m/z: (ESI+) MH⁺, 305.98.

b) A 50% aqueous solution of NaOH (7 mL) was added to 4-(2-chloro-6-(ethylsulfonylmethyl)pyrimidin-4-yl)morpholine (0.5 g, 1.81 mmol), 1,2-dibromoethane (0.156 mL, 1.81 mmol) and tetrabutylammonium bromide (0.058 g, 0.18 mmol) in DCM (21 mL). The resulting slurry was stirred at 30° C. for 18 hours and then evaporated. The residue was dissolved in EtOAc (30 mL) and washed with water (2×20 mL) and brine (20 mL), dried over MgSO₄ and then concentrated in vacuo. The residue was purified by chromatography on silica with an elution gradient of 20-60% EtOAc:DCM. Fractions containing product were combined and evaporated to afford 4-(2-chloro-6-(1-(ethylsulfonyl)cyclopropyl)pyrimidin-4-yl)morpholine (0.309 g. 57%); ¹H NMR (400 MHz, DMSO-d⁶) 1.26 (3H, t), 1.50-1.52 (2H, m), 1.60-1.62 (2H, m), 3.35 (2H, q), 3.60-3.68 (8H, m), 6.98 (1H, s); m/z: (ESI+) MH⁺, 332.02.

^{c}The 4-(2-chloro-6-(1-(isopropylsulfonyl)cyclopropyl)pyrimidin-4-yl)morpholine, used as starting material, was prepared as follows:

a) A 50% aqueous solution of NaOH (3.33 mL) was added to 4-(2-chloro-6-(isopropylsulfonylmethyl)pyrimidin-4-yl)morpholine (500 mg, 1.56 mmol), 1,2-dibromoethane (0.135 mL, 1.56 mmol) and tetrabutylammonium bromide (50.4 mg, 0.16 mmol) in DCM (10 mL). The resulting slurry was stirred at 30° C. for 24 hours and then evaporated. The residue was dissolved in EtOAc (30 mL) and the solution washed with water (2×20 mL) and then brine (20 mL) and then dried over MgSO₄ and concentrated in vacuo. The residue was purified by chromatography on silica with an elution gradient of 0 to 2% MeOH in DCM. Fractions containing product were combined and evaporated to afford 4-(2-chloro-6-(1-(isopropylsulfonyl)cyclopropyl)pyrimidin-4-yl)morpholine (486 mg, 90%); ¹H NMR (400 MHz, DMSO-d⁶) 1.28 (6H, d), 1.51-1.60 (4H, m), 3.57-3.69 (9H, m), 6.98 (1H, s); m/z: (ESI+) MH⁺, 346.01.

4-(2-Chloro-6-(isopropylsulfonylmethyl)pyrimidin-4-yl)morpholine used as starting material can be prepared as described in the literature (Finlay, Maurice Raymond Verschoyle; Morris, Jeffrey; Pike, Kurt Gordon. Morpholinopyrimidine derivatives, processes for preparing them, pharmaceutical compositions containing them, and their use for treating proliferative disorders. PCT Int. Appl. WO 2008023159).

^{d}The 4-(2-chloro-6-(1-(cyclopropylsulfonyl)cyclopropyl)pyrimidin-4-yl)morpholine, used as starting material, can be prepared as described in the literature (Morris, Jeffrey James; Pike, Kurt Gordon. Pyrimidine derivatives that are useful in the treatment of diseases mediated by mTOR and/or PI3K enzyme and their preparation. PCT Int. Appl. WO2009007748).
^{e}The 4-(2-chloro-6-(4-(cyclopropylsulfonyl)tetrahydro-2H-pyran-4-yl)pyrimidin-4-yl)morpholine, used as starting material, was prepared as follows:

a) A 50% aqueous solution of NaOH (6.67 mL) was added to 4-(2-chloro-6-(cyclopropylsulfonylmethyl)pyrimidin-4-yl)morpholine (700 mg, 2.20 mmol), tetrabutylammonium bromide (71.0 mg, 0.22 mmol) and 1-bromo-2-(2-bromoethoxy)ethane (1533 mg, 6.61 mmol) in DCM (20 mL) at 22° C. The resulting mixture was stirred at RT for 6 hours and then diluted with water (50 mL) and the phases separated. The organic phase was washed twice with water (25 mL) and the organic layer dried over MgSO4, filtered and evaporated. The residue was purified by chromatography on silica with an elution gradient of 0 to 20% EtOAc in DCM. Fractions containing product were combined and evaporated to afford 4-(2-chloro-6-(4-(cyclopropylsulfonyl)tetrahydro-2H-pyran-4-yl)pyrimidin-4-yl)morpholine (557 mg, 65%); ¹H NMR (400 MHz, DMSO-d⁶) 0.72-0.75 (2H, m), 0.87-0.92 (2H, m), 2.12-2.17 (2H, m), 2.65-2.69 (2H, m), 3.12-3.18 (2H, m), 3.63-3.64 (8H, m), 3.83-3.87 (2H, m), 7.04 (1H, s); m/z: (ESI+) MH⁺, 388.08.

The 4-(2-chloro-6-(cyclopropylsulfonylmethyl)pyrimidin-4-yl)morpholine, used as starting material, can be prepared as described in the literature (Morris, Jeffrey James; Pike, Kurt Gordon. Pyrimidine derivatives that are useful in the treatment of diseases mediated by mTOR and/or PI3K enzyme and their preparation. PCT Int. Appl. WO2009007748).

^{f}The tert-butyl 4-(2-chloro-6-morpholinopyrimidin-4-yl)-4-(cyclopropylsulfonyl)piperidine-1-carboxylate, used as starting material, was prepared as follows:

a) A solution of 4-(2-chloro-6-(cyclopropylsulfonylmethyl)pyrimidin-4-yl)morpholine (1 g, 3.15 mmol) in NMP (9 mL) was treated with sodium hydride (0.415 g, 10.38 mmol). The mixture was stirred at RT for 10 minutes before being treated with tetrabutylammonium bromide (1.522 g, 4.72 mmol) and N-benzyl-2-chloro-N-(2-chloroethyl)ethanamine hydrochloride (0.930 g, 3.46 mmol). The reaction mixture was stirred for 5 minutes at RT and then heated to 50° C. for 1 hour and then heated to 80° C. for 1.5 hours. The mixture was allowed to cool to RT and the mixture quenched by the addition of a saturated aqueous solution of ammonium chloride. The mixture was extracted with EtOAc and the organic solution washed three times with water and then dried over magnesium sulfate. The solution was concentrated under reduced pressure and the residue purified by chromatography on silica using a gradient elution of 10% EtOAc/DCM to 70% EtOAc/DCM. Fractions containing product were combined and evaporated to afford 4-(6-(1-benzyl-4-(cyclopropylsulfonyl)piperidin-4-yl)-2-chloropyrimidin-4-yl)morpholine (0.574 g, 38%); ¹H NMR (400 MHz, DMSO-d⁶) 0.75-0.77 (2H, m), 0.93-0.95 (2H, m), 1.81-1.92 (2H, m), 2.11-2.19 (2H, m), 2.53-2.56 (1H, m), 2.71 (2H, s), 2.79-2.82 (4H, m), 3.66-3.68 (8H, m), 7.00 (1H, s), 7.24-7.34 (5H, m); m/z: (ESI+) MH⁺, 477.13.

b) 1-Chloroethyl carbonochloridate (0.260 mL, 2.41 mmol) was added dropwise to a solution of 4-(6-(1-benzyl-4-(cyclopropylsulfonyl)piperidin-4-yl)-2-chloropyrimidin-4-yl)morpholine (574 mg, 1.20 mmol) in DCM (10 mL) at RT. The solution was heated at reflux for 3 hours and then allowed to cool and diluted with MeOH (10 mL). The mixture was allowed to stand for three days and then treated with di-tert-butyl dicarbonate (578 mg, 2.65 mmol) and N-ethyl-N-isopropylpropan-2-amine (0.419 mL, 2.41 mmol) and the solution was stirred for 2 hours. The mixture was diluted with DCM (20 mL), and the mixture washed with water (50 mL) and then with a saturated aqueous solution of brine (50 mL). The organic layer was dried over MgSO4, filtered and then evaporated. The residue was purified by chromatography on silica with an elution gradient of 10 to 50% EtOAc in DCM. Fractions containing product were combined and evaporated to afford tert-butyl 4-(2-chloro-6-morpholinopyrimidin-4-yl)-4-(cyclopropylsulfonyl)piperidine-1-carboxylate (267 mg, 46%); ¹H NMR (400 MHz, DMSO-d⁶) 0.77-0.78 (2H, m), 0.94-0.97 (2H, m), 1.40 (9H, s), 1.97-2.00 (2H, m), 2.54-2.57 (3H, m), 2.81-2.84 (2H, d), 3.69 (8H, s), 3.93-3.97 (2H, m), 7.01 (1H, s); m/z: (ESI+) MH⁺, 487.09.

### EXAMPLE 2.01

### 4-{4-[4-(Cyclopropylsulfonyl)piperidin-4-yl]-6-[(3S)-3-methylmorpholin-4-yl]pyrimidin-2-yl}-1H-indole

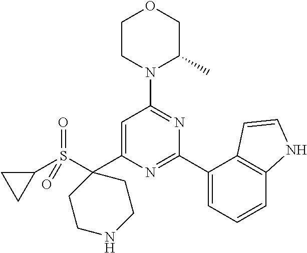

A mixture of bis(triphenylphosphine)palladium(II) dichloride (42.1 mg, 0.06 mmol), tert-butyl 4-[2-chloro-6-[(3S)-3-methylmorpholin-4-yl]pyrimidin-4-yl]-4-cyclopropylsulfonylpiperidine-1-carboxylate (301 mg, 0.6 mmol), Na₂CO₃ solution (1.8 mL, 2M, 3.60 mmol) and 1H-indol-4-ylboronic acid (116 mg, 0.72 mmol) in 18% DMF in 7:3:2 DME-water-EtOH (10 mL) was heated to 110° C. for 1 h in a microwave reactor. The mixture was part purified using an SCX column, eluting with 7M NH₃ in MeOH. Appropriate fractions were combined and concentrated in vacuo. The residue was dissolved in 4M HCl in dioxane, and the solution was stirred at RT for 1 h. The mixture was concentrated in vacuo and the residue was purified by preparative HPLC to provide the title compound; (27.0 mg, 9%); ¹H NMR: 0.78-0.90 (4H, m), 1.27 (3H, d), 1.88-1.95 (2H, m), 2.19 (2H, t), 2.40-2.48 (1H, m), 2.62-2.82 (1H, m), 3.18 (2H, t), 3.33-3.42 (2H, m), 3.52-3.58 (1H, m), 3.69-3.73 (1H, m), 3.83 (1H, d), 4.02-4.06 (1H, m), 4.30 (1H, d), 4.65 (1H, d), 6.97 (1H, s), 7.21 (1H, t), 7.29 (1H, t), 7.47 (1H, t), 7.56 (1H, d), 8.12-8.15 (1H, m), 11.29 (1H, s); m/z: (ESI+) MH⁺, 482.82.

The tert-butyl 4-[2-chloro-6-[(3S)-3-methylmorpholin-4-yl]pyrimidin-4-yl]-4-cyclopropylsulfonylpiperidine-1-carboxylate, used as starting material, was prepared as follows:

a) Sodium cyclopropanesulfinate (381 mg, 2.97 mmol) was added in one portion to 2-chloro-4-(iodomethyl)-6-[(3S)-3-methylmorpholin-4-yl]pyrimidine (700 mg, 1.98 mmol) in acetonitrile (20 mL). The resulting suspension was heated to 90° C. for 3 h. The mixture was then concentrated in vacuo and the residue was dissolved in DCM (50 mL). Water (50 mL) was added and the phases were separated. The organic portion was dried (MgSO₄) and concentrated in vacuo. Purification by FCC, using a gradient of 0-40% EtOAc in DCM provided 2-chloro-4-(cyclopropylsulfonylmethyl)-6-[(3S)-3-methylmorpholin-4-yl]pyrimidine (458 mg); ¹H NMR: 0.95-0.98 (2H, m), 1.02-1.06 (2H, m), 1.18-1.23 (3H, m), 2.77-2.83 (1H, m), 3.19-3.25 (1H, m), 3.42-3.49 (1H, m), 3.58-3.62 (1H, m), 3.73 (1H, d), 3.92-3.96 (2H, m), 4.30 (1H, s), 4.48 (2H, s), 6.92 (1H, s); m/z: (ESI+) MH⁺, 332.

b) NaH (0.796 g, 19.89 mmol) was added to a solution of 2-chloro-4-(cyclopropylsulfonylmethyl)-6-[(3S)-3-methylmorpholin-4-yl]pyrimidine (2 g, 6.03 mmol) in NMP (18 mL) and the mixture was stirred for 10 minutes. TBAB (2.91 g, 9.04 mmol) and N-benzyl-2-chloro-N-(2-chloroethyl)ethanamine hydrochloride (1.781 g, 6.63 mmol) were then added and the mixture was stirred for 5 minutes. The mixture was then heated to 50° C. for 1 h then to 80° C. for 1.5 h. After cooling to RT the reaction was quenched by the addition of saturated NH₄Cl solution. The mixture was then extracted with EtOAc. The organic solution was washed three times with water and was then dried (MgSO₄), and concentrated in vacuo. Purification by FCC using a gradient of 10-70% EtOAc in DCM provided 4-(1-benzyl-4-cyclopropylsulfonyl-piperidin-4-yl)-2-chloro-6-[(3S)-3-methylmorpholin-4-yl]pyrimidine (2.23 g); ¹H NMR: (CDCl₃) 0.92-0.96 (2H, m), 0.97-1.02 (2H, m), 1.32 (3H, d), 1.92-2.00 (2H, m), 2.24-2.31 (1H, m), 2.40-2.49 (2H, m), 2.68-2.74 (2H, m), 2.88-2.92 (2H, m), 3.29 (1H, dt), 3.40 (2H, s), 3.55 (1H, dt), 3.70 (1H, dd), 3.79 (1H, d), 3.98-4.09 (2H, m), 4.28 (1H, bs), 6.63 (1H, s), 7.21-7.33 (5H, m); m/z: (ESI+) MH⁺, 491 and 493.

c) 1-Chloroethyl chloroformate (0.971 mL, 9.00 mmol) was added to a solution of 4-(1-benzyl-4-cyclopropylsulfonylpiperidin-4-yl)-2-chloro-6-[(3S)-3-methylmorpholin-4-yl]pyrimidine (2.21 g, 4.50 mmol) in DCM (15 mL). The solution was heated to reflux for 1.5 h. The mixture was then diluted with MeOH (15 mL) and heating was continued for 2 h. Di-tert-butyl dicarbonate (2.16 g, 9.90 mmol) and DIPEA (1.6 mL, 9.0 mmol) were then added and the mixture was stirred at RT for 1 h. The mixture was then partitioned between DCM and water and the phases were separated. The organic portion was concentrated in vacuo. Purification by FCC using a gradient of 10-30% EtOAc in DCM provided tert-butyl 4-[2-chloro-6-[(3S)-3-methylmorpholin-4-yl]pyrimidin-4-yl]-4-cyclopropylsulfonylpiperidine-1-carboxylate (1.9 g); ¹H NMR: (CDCl₃) 0.93-1.00 (4H, m), 1.32 (3H, d), 1.44 (9H, s), 2.19-2.30 (3H, m), 2.62-2.80 (4H, m), 3.29 (1H, dt), 3.55 (1H, dt), 3.69 (1H, dd), 3.79 (1H, d), 3.95-4.37 (5H, m), 6.65 (1H, s); m/z: (ESI+) MH⁺, 501 and 503.

### EXAMPLE 2.02

### 4-{4-[4-(Cyclopropylsulfonyl)tetrahydro-2H-pyran-4-yl]-6-[(3S)-3-methylmorpholin-4-yl]pyrimidin-2-yl}-1H-indole

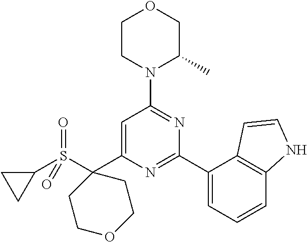

Prepared in a similar way to that described for example 2.01 starting from (S)-4-(2-chloro-6-(4-(cyclopropylsulfonyl)tetrahydro-2H-pyran-4-yl)pyrimidin-4-yl)-3-methylmorpholine (300 mg, 0.75 mmol) to afford the title compound (16 mg, 4%); ¹H NMR: 0.77-0.82 (2H, m), 0.88-0.89 (2H, m), 1.27 (3H, d), 2.25-2.32 (2H, td), 2.95 (1H, dd), 3.27-3.32 (4H, m), 3.35 (1H, d), 3.53 (1H, td), 3.69-3.73 (1H, m), 3.81 (1H, d), 3.95 (2H, t), 4.01 (1H, dd), 4.29 (1H, d), 4.63 (1H, d), 6.95 (1H, s), 7.20 (1H, t), 7.32 (1H, d), 7.46 (1H, t), 7.55 (1H, d), 8.11-8.14 (1H, m), 11.24 (1H, s); m/z: (ESI+) MH⁺, 483.37.

The (S)-4-(2-chloro-6-(4-(cyclopropylsulfonyl)tetrahydro-2H-pyran-4-yl)pyrimidin-4-yl)-3-methylmorpholine, used as starting material, can be prepared as described in the literature (Morris, Jeffrey James; Pike, Kurt Gordon. Pyrimidine derivatives that are useful in the treatment of diseases mediated by mTOR and/or PI3K enzyme and their preparation. PCT Int. Appl. (2009), WO2009007748).

### EXAMPLE 2.03

### 4-{4-[(3S)-3-Methylmorpholin-4-yl]-6-[1-(methylsulfonyl)cyclopentyl]pyrimidin-2-yl}-1H-indole

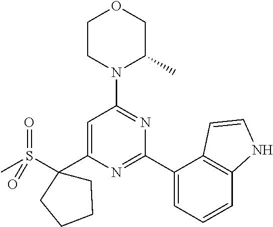

A mixture of bis(triphenylphosphine)palladium(II) dichloride (42.1 mg, 0.06 mmol), 2-chloro-4-[(3S)-3-methylmorpholin-4-yl]-6-(1-methylsulfonylcyclopentyl)-pyrimidine (216 mg, 0.6 mmol), and 1H-indol-4-ylboronic acid (116 mg, 0.72 mmol) in 18% DMF in 7:3:2 DME-water-EtOH (10 mL) was heated to 110° C. for 1 h in a microwave reactor. After cooling to RT, the mixture was part purified using an SCX column, eluting with 7M NH₃ in MeOH. Further purification by preparative HPLC provided the title compound (55 mg, 21%); ¹H NMR: 1.28 (3H, d), 1.60-1.65 (2H, m), 1.84-1.89 (2H, m), 2.48-2.55 (2H, m), 2.77-2.86 (2H, m), 2.90 (3H, s), 3.30-3.35 (1H, m), 3.51-3.58 (1H, m), 3.68-3.71 (1H, m), 3.81 (1H, d), 4.00-4.04 (1H, m), 4.26 (1H, d), 4.61 (1H, s), 6.87 (1H, s), 7.21 (1H, t), 7.35 (1H, t), 7.47 (1H, t), 7.55 (1H, d), 8.12-8.14 (1H, m), 11.26 (1H, s); m/z: (ESI+) MH⁺, 441.75.

The 2-chloro-4-[(3S)-3-methylmorpholin-4-yl]-6-(1-methylsulfonylcyclopentyl)pyrimidine, used as starting material, was prepared as follows:

a) Triethylamine (17.4 mL, 0.13 mol) was added to a cooled (−5° C.) DCM solution of 2,4-dichloro-6-(methylsulfonylmethyl)pyrimidine (30 g, 0.13 mol). A DCM solution of (3S)-3-methylmorpholine was then added dropwise, while keeping the temperature below −5° C. The cooling bath was then removed and the mixture stirred for 1 h. The mixture was then heated to reflux for 2 h. The mixture was then washed with water, dried and concentrated in vacuo. Purification by preparative HPLC provided 2-chloro-4-[(3S)-3-methylmorpholin-4-yl]-6-(methylsulfonylmethyl)-pyrimidine (19.3 g); ¹H NMR: 1.21-1.23 (m, 3H), 3.11 (s, 3H), 3.19-3.26 (m, 1H), 3.42-3.49 (m, 1H), 3.58-3.62 (1H, m), 3.73 (d, 1H), 3.92-3.96 (m, 2H), 4.27-4.31 (m, 1H), 4.45 (s, 2H), 6.92 (s, 1H); m/z: (ESI+) MH⁺, 306.

Alternatively 2-chloro-4-[(3S)-3-methylmorpholin-4-yl]-6-(methylsulfonylmethyl)-pyrimidine can be prepared by the following method:

Sodium methanesulfinate (11.75 g, 115.11 mmol) was added in one portion to 2-chloro-4-(iodomethyl)-6-[(3S)-3-methylmorpholin-4-yl]pyrimidine (37 g, 104.64 mmol), in MeCN (900 mL) and the resulting solution was stirred at 85° C. for 24 h and then cooled to RT. The reaction mixture was evaporated to dryness and redissolved in DCM (500 ml), washed with water (3×100 ml), and brine (100 ml), dried over MgSO4, filtered and concentrated in vacuo. Purification by FCC using a gradient of 0-30% EtOAc in DCM provided 2-chloro-4-[(3S)-3-methylmorpholin-4-yl]-6-(methylsulfonylmethyl)pyrimidine (22 g) as a solid.

b) TBAB (0.495 g, 1.54 mmol) was added to a mixture of 2-chloro-4-[(3S)-3-methylmorpholin-4-yl]-6-(methylsulfonylmethyl)pyrimidine (4.7 g, 15.37 mmol), 1,4-dibromobutane (1.84 mL, 15.37 mmol) and aqueous NaOH (30 mL, 368.9 mmol) in DCM (150 mL). The resulting mixture was heated to 40° C. for 6 h. The mixture was then diluted with DCM (200 mL), and washed with water (100 mL). The organic solution was dried (MgSO₄) and concentrated in vacuo. Purification by FCC using a gradient of 5-50% EtOAc in isohexane provided 2-chloro-4-[(3S)-3-methylmorpholin-4-yl]-6-(1-methylsulfonylcyclopentyl)pyrimidine (3.90 g); ¹H NMR: 1.20 (3H, d), 1.50-1.60 (2H, m), 1.72-1.82 (2H, m), 2.30-2.41 (2H, m,), 2.50-2.60 (2H, m), 2.88 (3H, s), 3.20 (1H, dd), 3.45 (1H, dd), 3.60 (1H, dd), 3.71 (1H, d), 3.94 (1H, dd), 4.0-4.10 (1H, m), 4.42 (1H, s), 6.89 (1H, s); m/z: (ESI+) MH⁺, 360.

The following compounds were prepared in a similar way to the method described for Example 2.03, using the appropriate 2-chloro pyrimidine derivative and 1H-indol-4-ylboronic acid.

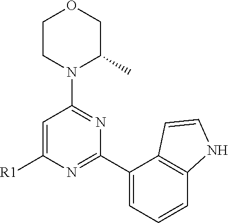

| | | | | m/z |
|---|---|---|---|---|
| Ex. | | | | (ESI+) |
| No. | R1 | Name | ¹H NMR | MH⁺ |
| 2.04^{a} | 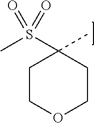 | 4-{4-[(3S)-3- Methylmorpholin- 4-yl]-6-[4- (methylsulfonyl) tetrahydro-2H- pyran-4- yl]pyrimidin-2- yl}-1H-indole | 1.28 (3H, d), 2.23-2.34 (2H, m), 2.68-2.71 (1H, m), 2.86-2.91 (4H, m), 3.20-3.25 (3H, m), 3.54-3.57 (1H, m), 3.69-3.73 (1H, m), 3.81 (1H, d), 3.94-4.05 (3H, m), 4.31 d), 4.64 (1H, d), 6.93 (1H, s), 7.20 (1H, t), 7.28 (1H, t), 7.46 (1H, t), 7.55 (1H, d), 8.10-8.12 (1H, m), 11.26 (1H, s) | 457.77 |
| 2.05^{b} | 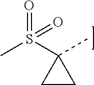 | 4-{4-[(3S)-3- Methylmorpholin- 4-yl]-6-[1- (methylsulfonyl)- cyclopropyl] pyrimidin-2-yl)-1H- indole^{#} | 1.28 (3H, d), 1.61-1.63 (2H, m), 1.71-1.73 (2H, m), 3.25-3.30 (4H, m), 3.50-3.54 (1H, m), 3.69 (1H, d), 3.79-3.82 (1H, m), 4.02-4.05 (1H, m), 4.18-4.22 (1H, m), 4.57- 4.59 (1H, m), 6.85 (1H, s), 7.21 (1H, t), 7.29-7.31 (1H, m), 7.44- 7.46 (1H, m), 7.55 (1H, d), 8.04 (1H, d), 11.25 (1H, s) | 413.12 |

# Chiral HPLC: (HP1100 System 4, 5 μm Chiralpak AS-H (250 mm×4.6 mm) column eluting with iso-Hexane/EtOH/TEA 60/40/0.1) Rf, 11.817 >99%.
^{a}The 2-chloro-4-[(3S)-3-methylmorpholin-4-yl]-6-(4-methylsulfonyloxan-4-yl)pyrimidine, used as starting material, was prepared as follows:

Sodium tert-butoxide (1.38 g, 14.39 mmol) was added portionwise over a period of 10 minutes to a cooled (0° C.) mixture of 2-chloro-4-[(3S)-3-methylmorpholin-4-yl]-6-(methylsulfonylmethyl)pyrimidine (2.00 g, 6.54 mmol) and bis(2-bromoethyl) Et2O (2.055 mL, 16.35 mmol) in DMF (75 mL). The resulting solution was allowed to warm to RT and was stirred for 7 h. Further sodium tert-butoxide (629 mg, 6.54 mmol) was added portionwise and the solution was then stirred at RT for a further 45 h. The mixture was then concentrated in vacuo and diluted with EtOAc (200 mL). The solution was washed with water (2×200 mL) and then saturated brine (100 mL). The solution was then dried (MgSO₄) and concentrated in vacuo. Purification by FCC using a gradient of 40-100% EtOAc in isohexane and crystallisation using EtOAc and isohexane provided 2-chloro-4-[(3S)-3-methylmorpholin-4-yl]-6-(4-methylsulfonyloxan-4-yl)pyrimidine (1.42 g); ¹H NMR: (CDCl₃) 1.34 (3H, d), 2.50 (2H, m), 2.55 (2H, m), 2.73 (3H, s), 3.33 (3H, m), 3.56 (1H, ddd), 3.71 (1H, dd), 3.80 (1H, d), 4.01 (4H, m), 4.31 (1H, bs), 6.62 (1H, s); m/z: (ESI+) MH⁺, 376 and 378.

^{b}The 2-chloro-4-[(3S)-3-methylmorpholin-4-yl]-6-(1-methylsulfonylcyclopropyl)-pyrimidine, used as starting material was prepared as follows:

i) 2-Chloro-4-[(3S)-3-methylmorpholin-4-yl]-6-(methylsulfonylmethyl)-pyrimidine (1.2 g, 3.9 mmol) was dissolved in DMF (20 mL). Sodium tert-butoxide (755 mg, 7.85 mmol) was added to the mixture, followed by dibromoethane (738 mg, 3.9 mmol). The mixture was stirred for 4 h and was then heated to 60° C. overnight. Further sodium tert-butoxide (378 mg, 3.9 mmol) was then added, followed by dibromoethane (369 mg, 1.9 mmol) and the mixture was maintained at 60° C. for a further 24 h. DCM (20 mL) was then added and the solution was washed with 2M aqueous HCl (20 mL). The organic solution was dried (MgSO₄) and concentrated in vacuo. Purification by FCC using a gradient of 0-50% EtOAc in DCM provided 2-chloro-4-[(3S)-3-methylmorpholin-4-yl]-6-(1-methylsulfonylcyclopropyl)-pyrimidine (400 mg, 31%); ¹H NMR: 1.22 (d, 3H), 1.51 (m, 2H), 1.64 (m, 2H), 3.18 (s, 3H), 3.22 (m, 1H), 3.43 (m, 1H), 3.58 (m, 1H), 3.72 (d, 1H), 3.93 (m, 1H), 4.05 (d, 1H), 4.41 (s, 1H), 6.93 (s, 1H); m/z: (ESI+) MH⁺332.

Alternatively, 2-chloro-4-[(3S)-3-methylmorpholin-4-yl]-6-(1-methylsulfonyl-cyclopropyl)pyrimidine, can be prepared as follows:

NaOH (50% w/w solution, 115 g, 2.878 mol) was added to 2-chloro-4-[(3S)-3-methylmorpholin-4-yl]-6-(methylsulfonylmethyl)pyrimidine (16 g, 52.33 mmol), 1,2-dibromoethane (13.53 mL, 156.98 mmol) and TBAB (1.687 g, 5.23 mmol) in toluene (128 mL). The resulting suspension was stirred for 4 h. Water was then added and the mixture was extracted twice with toluene. The toluene extracts were dried (MgSO₄) and concentrated in vacuo. Purification by FCC using a gradient of 0-20% EtOAc in DCM provided 2-chloro-4-[(3S)-3-methylmorpholin-4-yl]-6-(1-methylsulfonyl-cyclopropyl)pyrimidine (13 g) as a solid.

### EXAMPLE 3.01

### 4-{4-[(3R)-3-Methylmorpholin-4-yl]-6-[1-(methylsulfonyl)cyclopropyl]pyrimidin-2-yl}-1H-indole

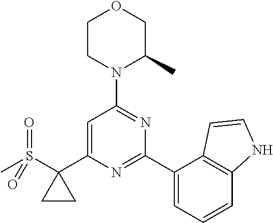

Bis(triphenylphosphine)palladium chloride (1.692 g, 2.41 mmol), (R)-4-(2-chloro-6-(1-(methylsulfonyl)cyclopropyl)pyrimidin-4-yl)-3-methylmorpholine (8.00 g, 24.11 mmol), 1H-indol-4-ylboronic acid (4.27 g, 26.52 mmol) and 2M aqueous sodium carbonate (36.2 mL, 72.33 mmol) were suspended in DME:water 4:1 (170 mL) and heated to 90° C. overnight. The DME was removed and the reaction mixture diluted with EtOAc (100 mL). The mixture was washed with water (2×100 mL), the organics separated, filtered through a pad of Celite and concentrated in vacuo on to silica. The residue was purified by chromatography on silica with an elution gradient of 0 to 10% EtOAc in DCM. Fractions containing product were combined and evaporated. The residue was purified by chromatography on silica eluting with a gradient of 0-25% EtOAc in DCM. Fractions containing product were combined and evaporated onto reverse phase C18 silica. The crude product was purified by reverse phase using a 415 g HP C18 column using decreasingly polar mixtures of water (containing 1% NH3) and MeCN as eluents. Fractions containing product were combined and evaporated. The residue was taken up in dry MeOH and dried over MgSO4. The mixture was filtered and the solvent evaporated, leaving a gum. The gum was dissolved in DCM (500 mL), filtered and the solvent removed under reduced pressure. The residue was dissolved in MeOH (50 mL) and allowed to stir at RT overnight. The resultant precipitate was collected by filtration to afford the title compound (5.10 g, 51%); ¹H NMR (400 MHz, DMSO-d⁶): 1.29 (3H, d), 1.57-1.64 (2H, m), 1.68-1.78 (2H, m), 3.24-3.31 (1H, td), 3.29 (3H, s), 3.51 (1H, td), 3.67 (1H, dd), 3.80 (1H, d), 3.93-4.06 (1H, dd), 4.21 (1H, d), 4.61 (1H, bs), 6.85 (1H, s), 7.21 (1H, t), 7.32 (1H, t), 7.46 (1H, t), 7.56 (1H, d), 8.06 (1H, dd), 11.25 (1H, s); m/z: (ESI+) MH⁺, 413.12. Chiral HPLC: (HP1100 System 4, 5 μm Chiralpak AS-H (250 mm×4.6 mm) column eluting with iso-Hexane/EtOH/TEA 60/40/0.1) Rf, 8.815 >99%.

The (R)-4-(2-chloro-6-(1-(methylsulfonyl)cyclopropyl)pyrimidin-4-yl)-3-methylmorpholine, used as starting material, was prepared as follows:

a) (R)-3-methylmorpholine (7.18 g, 71.01 mmol) and triethylamine (12.87 mL, 92.31 mmol) were added to methyl 2,4-dichloropyrimidine-6-carboxylate (14.70 g, 71.01 mmol) in DCM (100 mL). The resulting mixture was stirred at RT for 18 hours. Water (100 mL) was added, the layers separated and extracted with DCM (5 mL). The combined organics were dried over MgSO4, concentrated in vacuo and the residue triturated with Et2O to yield (R)-methyl 2-chloro-6-(3-methylmorpholino)pyrimidine-4-carboxylate (14.77 g, 77%); ¹H NMR (400 MHz, CDCl₃) 1.33-1.37 (3H, d), 3.31-3.38 (1H, m), 3.52-3.59 (1H, m), 3.68-3.72 (1H, m), 3.79-3.83 (1H, m), 3.98 (3H, s), 4.02-4.05 (1H, m), 4.12 (1H, br s), 4.37 (1H, br s), 7.16 (1H, s); m/z: (ESI+) MH⁺, 272.43.

The liquors were concentrated onto silica and purified by chromatography on silica eluting with a gradient of 20 to 40% EtOAc in isohexane. Fractions containing product were combined and evaporated to afford (R)-methyl 2-chloro-6-(3-methylmorpholino)pyrimidine-4-carboxylate (1.659 g, 9%); ¹H NMR (400 MHz, CDCl₃) 1.33-1.37 (3H, d), 3.31-3.38 (1H, m), 3.52-3.59 (1H, m), 3.68-3.72 (1H, m), 3.79-3.83 (1H, m), 3.98 (3H, s), 4.02-4.05 (1H, m), 4.12 (1H, br s), 4.37 (1H, br s), 7.16 (1H, s); m/z: (ESI+) MH⁺, 272.43.

b) Lithium borohydride, 2M in THF (18 mL, 36.00 mmol) was added dropwise to (R)-methyl 2-chloro-6-(3-methylmorpholino)pyrimidine-4-carboxylate (16.28 g, 59.92 mmol) in THF (200 mL) at 0° C. over a period of 20 minutes under nitrogen. The resulting solution was stirred at 0° C. for 30 minutes and then allowed to warm to RT and stirred for a further 18 hours. Water (200 mL) was added and the THF evaporated. The aqueous layer was extracted with EtOAc (2×100 mL) and the organic phases combined, dried over MgSO4 and then evaporated to afford (R)-(2-chloro-6-(3-methylmorpholino)pyrimidin-4-yl)MeOH (14.54 g, 100%) which was used in the next step without purification; ¹H NMR (400 MHz, CDCl₃) 1.32 (3H, d), 2.65 (1H, br s), 3.25-3.32 (1H, m), 3.51-3.57 (1H, m), 3.67-3.70 (1H, m), 3.78 (1H, d), 3.98-4.09 (2H, m), 4.32 (1H, br s), 4.59 (2H, s), 6.44 (1H, s); m/z: (ESI+) MH⁺, 244.40.

c) Methanesulfonyl chloride (4.62 mL, 59.67 mmol) was added dropwise to (R)-(2-chloro-6-(3-methylmorpholino)pyrimidin-4-yl)MeOH (14.54 g, 59.67 mmol) and triethylamine (8.32 mL, 59.67 mmol) in DCM (250 mL) at 25° C. over a period of 5 minutes. The resulting solution was stirred at 25° C. for 90 minutes. The reaction mixture was quenched with water (100 mL) and extracted with DCM (2×100 mL). The organic phases were combined, dried over MgSO4, filtered and evaporated to afford (R)-(2-chloro-6-(3-methylmorpholino)pyrimidin-4-yl)methyl methanesulfonate (20.14 g, 105%) which was used in the next step without further purification; ¹H NMR (400 MHz, CDCl₃) 1.33 (3H, d), 3.13 (3H, s), 3.27-3.34 (1H, m), 3.51-3.57 (1H, m), 3.66-3.70 (1H, m), 3.79 (1H, d), 3.99-4.03 (2H, m), 4.34 (1H, br s), 5.09 (2H, d), 6.52 (1H, s); m/z: (ESI+) MH⁺, 322.39.

d) Lithium iodide (17.57 g, 131.27 mmol) was added to (R)-(2-chloro-6-(3-methylmorpholino)pyrimidin-4-yl)methyl methanesulfonate (19.2 g, 59.67 mmol) in dioxane (300 mL) and heated to 100° C. for 2 hours under nitrogen. The reaction mixture was quenched with water (200 mL) and extracted with EtOAc (3×200 mL). The organic layers were combined and washed with 2M sodium bisulfite solution (400 mL), water (400 mL), brine (400 mL) dried over MgSO4 and then evaporated. The residue was triturated with Et2O to afford (R)-4-(2-chloro-6-(iodomethyl)pyrimidin-4-yl)-3-methylmorpholine (13.89 g, 66%); ¹H NMR (400 MHz, CDCl₃) 1.32 (3H, d), 3.24-3.32 (1H, m), 3.51-3.58 (1H, m), 3.67-3.71 (1H, m), 3.78 (1H, d), 3.98-4.02 (2H, m), 4.21 (2H, s), 4.29 (1H, s), 6.41 (1H, s); m/z: (ESI+) MH⁺354.31.

The mother liquors were concentrated down and triturated with Et2O to afford a further crop of (R)-4-(2-chloro-6-(iodomethyl)pyrimidin-4-yl)-3-methylmorpholine (2.46 g, 12%); ¹H NMR (400 MHz, CDCl₃) 1.32 (3H, d), 3.24-3.32 (1H, m), 3.51-3.58 (1H, m), 3.67-3.71 (1H, m), 3.78 (1H, d), 3.98-4.02 (2H, m), 4.21 (2H, s), 4.29 (1H, s), 6.41 (1H, s); m/z: (ESI+) MH⁺, 354.31.

e) Sodium methanesulfinate (4.64 g, 45.48 mmol) was added in one portion to (R)-4-(2-chloro-6-(iodomethyl)pyrimidin-4-yl)-3-methylmorpholine (13.4 g, 37.90 mmol) in DMF (100 mL). The resulting mixture was stirred at 25° C. for 2 hours. The reaction mixture was diluted with DCM and washed with water (2×100 mL), Aq sodium thiosulphate (50 mL), dried over MgSO4 and concentrated in vacuo. The residue was triturated with MeOH to give a solid which was dried under vacuum to afford (R)-4-(2-chloro-6-(methylsulfonylmethyl)pyrimidin-4-yl)-3-methylmorpholine (7.3 g, 63%); ¹H NMR (400 MHz, DMSO-d₆) 1.21 (3H, d), 3.12 (3H, s), 3.24 (1H, t), 3.42-3.49 (1H, td), 3.58-3.62 (1H, m), 3.74 (1H, d), 3.93-4.40 (3H, m), 4.47 (2H, s), 6.94 (1H, s); m/z: (ESI+) MH⁺306.05.

The mother liquers were purified by chromatography on silica with an elution gradient of 40 to 90% EtOAc in isohexane. Fractions containing product were combined and evaporated to afford a further crop of (R)-4-(2-chloro-6-(methylsulfonylmethyl)pyrimidin-4-yl)-3-methylmorpholine (2.0 g, 17%); m/z: (ESI+) MH⁺306.12.

f) A 50% aqueous solution of NaOH (42 mL) was added to (R)-4-(2-chloro-6-(methylsulfonylmethyl)pyrimidin-4-yl)-3-methylmorpholine (12.11 g, 39.60 mmol), 1,2-dibromoethane (3.41 mL, 39.60 mmol) and tetrabutylammonium bromide (1.277 g, 3.96 mmol) in toluene (120 mL). The resulting slurry was stirred at 60° C. for 3 hours and then an additional portion of 1,2-dibromoethane (1 mL) was added and the mixture stirred for a further 1 hour. EtOAc (200 mL) was added and the mixture washed with water (100 mL) and brine (100 mL). The reaction mixture was dried over MgSO₄ and concentrated in vacuo. The residue was triturated with MeOH to give a solid which was collected by filtration and dried under vacuum to afford (R)-4-(2-chloro-6-(1-(methylsulfonyl)cyclopropyl)pyrimidin-4-yl)-3-methylmorpholine (9.65 g, 73%); ¹H NMR (400 MHz, DMSO-d₆) 1.21 (3H, d), 1.48-1.54 (2H, m), 1.61-1.67 (2H, m), 3.16-3.25 (1H, td), 3.19 (3H, s), 3.44 (1H, td), 3.58 (1H, dd), 3.72 (1H, d), 3.93 (1H, dd), 3.98-4.10 (1H, m), 4.41 (1H, s), 6.93 (1H, s); m/z: (ESI+) MH⁺, 332.44.

The MeOH mother liquors were reduced in vacuo and the residue purified by chromatography on silica with an elution gradient of 10 to 50% EtOAc in DCM. Fractions containing product were combined and evaporated to afford (R)-4-(2-chloro-6-(1-(methylsulfonyl)cyclopropyl)pyrimidin-4-yl)-3-methylmorpholine (2.16 g, 16%); ¹H NMR (400 MHz, DMSO-d₆) 1.21 (3H, d), 1.49-1.54 (2H, m), 1.59-1.68 (2H, m), 3.19 (3H, s), 3.16-3.25 (1H, td), 3.44 (1H, td), 3.58 (1H, dd), 3.71 (1H, d), 3.93 (1H, dd), 4.04 (1H, bs), 4.41 (1H, bs), 6.93 (1H, s); m/z: (ESI+) MH⁺, 332.44.

### EXAMPLE 3.02

### 6-Methyl-4-{4-[(3R)-3-methylmorpholin-4-yl]-6-[1-(methylsulfonyl)cyclopropyl]pyrimidin-2-yl}-1H-indole

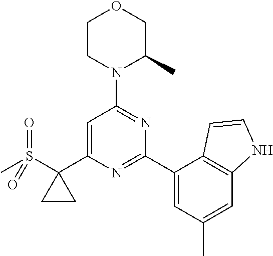

Tris(dibenzylideneacetone)dipalladium(0) (6.54 mg, 7.14 μmol) and tricyclohexylphosphine (10.68 mg, 0.04 mmol) were added to 4-bromo-6-methyl-1H-indole (100 mg, 0.48 mmol), potassium acetate (70.1 mg, 0.71 mmol) and bis(pinacolato)diboron (133 mg, 0.52 mmol) in dioxane (5 mL) under nitrogen. The resulting suspension was stirred at 90° C. for 3 hours. (R)-4-(2-Chloro-6-(1-(methylsulfonyl)cyclopropyl)pyrimidin-4-yl)-3-methylmorpholine (158 mg, 0.48 mmol), tetrakis(triphenylphosphine)palladium(0) (27.5 mg, 0.02 mmol) and sodium carbonate (2M aqueous solution) (0.952 mL, 1.90 mmol) were added and the resulting suspension was stirred at 90° C. for 18 hours. The reaction mixture was purified by ion exchange chromatography, using an SCX column. The product was eluted from the column using 20% 7M NH3/MeOH in DCM; fractions containing product were combined and evaporated. The residue was purified by preparative HPLC using decreasingly polar mixtures of water (containing 1% NH3) and MeCN as eluents. Fractions containing product were combined and evaporated to dryness to afford the title compound (50 mg, 25%); ¹H NMR (400 MHz, DMSO-d₆) 1.28 (3H, d), 1.59-1.64 (2H, m), 1.70-1.74 (2H, m), 2.47 (3H, s), 3.22-3.27 (1H, m), 3.28 (3H, s), 3.52 (1H, td), 3.67 (1H, dd), 3.80 (1H, d), 4.02 (1H, dd), 4.18-4.26 (1H, m), 4.56-4.65 (1H, m), 6.84 (1H, s), 7.21-7.24 (1H, m), 7.33-7.37 (2H, m), 7.87 (1H, s), 11.08 (1H, s); m/z: (ESI+) MH⁺427.19.

### EXAMPLE 3.03

### 2-Methyl-4-{4-[(3R)-3-methylmorpholin-4-yl]-6-[1-(methylsulfonyl)cyclopropyl]pyrimidin-2-yl}-1H-indole

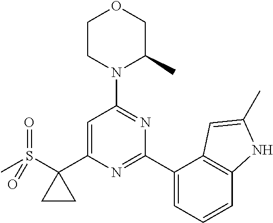

1,1′-Bis(diphenylphosphino)ferrocene-palladium dichloride (17.68 mg, 0.02 mmol) was added to a degassed solution of 2-methyl-1H-indol-4-yl trifluoromethanesulfonate (120 mg, 0.43 mmol), bis(pinacolato)diboron (115 mg, 0.45 mmol), 1,1′-bis(diphenylphosphino)ferrocene (12.04 mg, 0.02 mmol) and potassium acetate (127 mg, 1.29 mmol) in dioxane (5 mL) under nitrogen and the resulting mixture was stirred at 90° C. for 5 hours. (R)-4-(2-Chloro-6-(1-(methylsulfonyl)cyclopropyl)pyrimidin-4-yl)-3-methylmorpholine (143 mg, 0.43 mmol), tetrakis(triphenylphosphine)palladium(0) (24.83 mg, 0.02 mmol) and sodium carbonate (2M aqueous solution) (1.000 mL, 2 mmol) were added and heating continued for 24 hours. The reaction mixture was then purified by ion exchange chromatography, using an SCX column. The desired product was eluted from the column using 20% 7M NH3/MeOH in DCM and fractions containing product were combined and evaporated. The residue was purified by preparative HPLC using decreasingly polar mixtures of water (containing 1% NH3) and MeCN as eluents. Fractions containing product were combined and evaporated to afford the title compound (40 mg, 22%); ¹H NMR (400 MHz, DMSO-d₆) 1.29 (3H, d), 1.63-1.59 (2H, m), 1.74-1.70 (2H, m), 2.44 (3H, s), 3.27-3.23 (1H, m), 3.29 (3H, s), 3.52 (1H, td), 3.66 (1H, dd), 3.80 (1H, d), 4.02 (1H, dd), 4.24-4.16 (1H, m), 4.65-4.58 (1H, m), 6.82 (1H, s), 7.02-7.00 (1H, m), 7.10 (1H, t), 7.40 (1H, d), 8.00 (1H, dd), 11.07 (1H, s); m/z: (ESI+) MH⁺427.19.

### EXAMPLE 3.04

### 4-{4-[(3R)-3-Methylmorpholin-4-yl]-6-[1-(methylsulfonyl)cyclopropyl]pyrimidin-2-yl}-1H-indole-6-carbonitrile

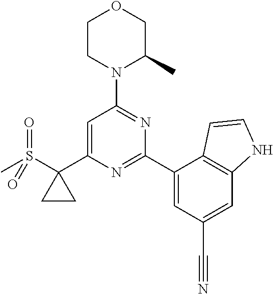

1,1′-Bis(diphenylphosphino)ferrocenedichloro palladium(II) dichloromethane complex (45.1 mg, 0.06 mmol) was added to 4-bromo-1H-indole-6-carbonitrile (100 mg, 0.45 mmol), potassium acetate (81 mg, 0.82 mmol) and bis(pinacolato)diboron (125 mg, 0.49 mmol) in dioxane (5 mL) under nitrogen. The resulting suspension was stirred at 90° C. for 3 hours. (R)-4-(2-Chloro-6-(1-(methylsulfonyl)cyclopropyl)pyrimidin-4-yl)-3-methylmorpholine (136 mg, 0.41 mmol), tetrakis(triphenylphosphine)palladium(0) (47.5 mg, 0.04 mmol) and sodium carbonate (2M aqueous solution) (0.823 mL, 1.65 mmol) were added and the resulting suspension was stirred at 90° C. for 18 hours. The reaction mixture was purified by ion exchange chromatography, using an SCX column. The desired product was eluted from the column using 20% 7M NH3/MeOH in DCM; fractions containing product were combined and evaporated. The residue was purified by chromatography on silica eluting with a gradient of 5 to 50% EtOAc in isohexane. Fractions containing product were combined and evaporated to afford the title compound (120 mg, 67%); ¹H NMR (400 MHz, DMSO-d₆) 1.29 (3H, d), 1.61-1.65 (2H, m), 1.71-1.76 (2H, m), 3.25 (3H, s), 3.27-3.29 (1H, m), 3.53 (1H, td), 3.67 (1H, dd), 3.81 (1H, d), 4.02 (1H, dd), 4.23 (1H, d), 4.57-4.65 (1H, m), 6.93 (1H, s), 7.44-7.47 (1H, m), 7.79 (1H, t), 8.03-8.05 (1H, m), 8.27 (1H, d), 11.86 (1H, s); m/z: (ESI+) MH⁺438.17.

The following compounds were prepared in a similar way to the method described for Example 3.04, using (R)-4-(2-chloro-6-(1-(methylsulfonyl)cyclopropyl)pyrimidin-4-yl)-3-methylmorpholine and the appropriate substituted 4-bromoindole.

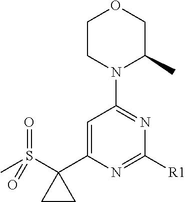

| | | | | m/z |
|---|---|---|---|---|
| Ex. | | | | (ESI+) |
| No. | R1 | Name | ¹H NMR | MH⁺ |
| 3.05 | 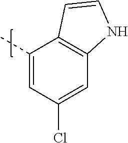 | 6-chloro-4-{4-[(3R)-3- methylmorpholin-4-yl}-6-[1- (methylsulfonyl)cyclopropyl]pyrimidin- 2-yl}-1H-indole | 1.29 (3H, d), 1.60-1.64 (2H, m), 1.71-1.75 (2H, m), 3.26 (3H, s), 3.27-3.30 (1H, m), 3.53 (1H, td), 3.67 (1H, dd), 3.81 (1H, d), 4.01 (1H, dd), 4.20 (1H, d), 4.55-4.64 (1H, m), 6.90 (1H, s), 7.32 (1H, t), 7.52 (1H, t), 7.59 (1H, dd), 8.00 (1H, d), 11.40 (1H, s) | 447.13 |
| 3.06 | 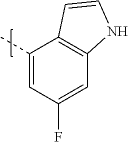 | 6-fluoro-4-{4-[(3R)-3- methylmorpholin-4-yl}-6-[1- (methylsulfonyflcyclopropyl]pyrimidin- 2-yl}-1H-indole | 1.28 (3H, d), 1.60-1.65 (2H, m), 1.71-1.76 (2H, m), 3.24- 3.29 (4H, m), 3.53 (1H, td), 3.67 (1H, dd), 3.81 (1H, d), 4.01 (1H, dd), 4.22 (1H, d), 4.55-4.65 (1H, m), 6.89 (1H, s), 7.31-7.34 (1H, m), 7.34-7.37 (1H, m), 7.47 (1H, t), 7.83 (1H, dd), 11.32 (1H, s) | 431.12 |
| 3.07 | 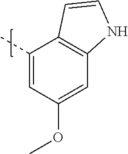 | 6-methoxy-4-{4-[(3R)-3- methylmorphohn-4-yl]-6-[1- (methylsulfonyl)cyclopropyl]pyrimidin- 2-yl}-1H-indole | 1.28 (3H, d), 1.60-1.63 (2H, m), 1.70-1.75 (2H, m), 3.23- 3.29 (4H, m), 3.52 (1H, td), 3.67 (1H, dd), 3.80 (1H, d), 3.83 (3H, s), 4.02 (1H, dd), 4.20 (1H, d), 4.57-4.64 (1H, m), 6.85 (1H, s), 7.08 (1H, d), 7.20 (1H, t), 7.31 (1H, t), 7.70 (1H, d), 11.04 (1H, s) | 443.14 |

### EXAMPLE 3.08

### 4-{4-[(3R)-3-Methylmorpholin-4-yl]-6-[4-(methylsulfonyl)tetrahydro-2H-pyran-4-yl]pyrimidin-2-yl}-1H-indole

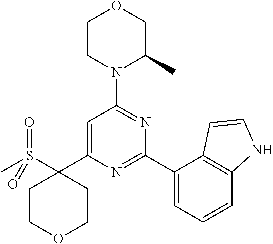

Dichlorobis(triphenylphosphine)palladium(11) (5.00 mg, 7.13 μmol) was added in one portion to (R)-4-(2-chloro-6-(4-(methylsulfonyl)tetrahydro-2H-pyran-4-yl)pyrimidin-4-yl)-3-methylmorpholine (268 mg, 0.71 mmol), 2M aqueous sodium carbonate solution (0.428 mL, 0.86 mmol) and 1H-indol-4-ylboronic acid (126 mg, 0.78 mmol) in DME:water 4:1 (10 mL) at 22° C. and sealed into a microwave tube. The mixture was heated to 110° C. for 1 hour in a microwave reactor and then cooled to RT. The crude product was purified by ion exchange chromatography, using an SCX column. The desired product was eluted from the column using 7M NH3/MeOH and fractions containing product were combined and evaporated. The residue was dissolved in DMF (2 mL) and then purified by preparative HPLC using decreasingly polar mixtures of water (containing 1% ammonia) and 3:1 MeOH:MeCN as eluents. Fractions containing the desired compound were combined and purified by ion exchange chromatography, using an SCX column. The desired product was eluted from the column using 7M NH3/MeOH. Fractions containing the desired compound were combined and evaporated to afford the title compound (111 mg, 34%); ¹H NMR (400 MHz, CDCl₃) 1.40-1.41 (3H, sm), 2.55-2.63 (2H, m), 2.73 (3H, s), 2.80-2.82 (2H, m), 3.38-3.53 (3H, m), 3.62-3.69 (1H, m), 3.78-3.89 (2H, m), 4.05-4.11 (3H, m), 4.23 (1H, d), 4.53-4.55 (1H, m), 6.70 (1H, s), 7.28-7.40 (3H, m), 7.53-7.56 (1H, m), 8.20-8.22 (1H, m), 8.56 (1H, s); m/z: (ESI+) MH⁺, 457.14.

The (R)-4-(2-chloro-6-(4-(methylsulfonyl)tetrahydro-2H-pyran-4-yl)pyrimidin-4-yl)-3-methylmorpholine, used as starting material, was prepared as follows:

A 50% aqueous NaOH solution (6.67 mL) was added to (R)-4-(2-chloro-6-(methylsulfonylmethyl)pyrimidin-4-yl)-3-methylmorpholine (380 mg, 1.24 mmol), tetrabutylammonium bromide (40.1 mg, 0.12 mmol) and 1-bromo-2-(2-bromoethoxy)ethane (721 mg, 3.11 mmol) in DCM (20 mL) at 22° C. The resulting mixture was stirred at 20° C. for 6 hours and then DCM (50 mL) was added. The solution was dried over sodium sulfate, filtered and the solvent removed under reduced pressure. The residue was purified by ion exchange chromatography, using an SCX column. The desired product was eluted from the column using 7M NH3/MeOH. Fractions containing product were combined and evaporated to afford (R)-4-(2-chloro-6-(4-(methylsulfonyl)tetrahydro-2H-pyran-4-yl)pyrimidin-4-yl)-3-methylmorpholine (268 mg, 57%); m/z: (ESI+) MH⁺, 376.10.

### EXAMPLE 3.09

### 6-Methyl-4-{4-[(3R)-3-methylmorpholin-4-yl]-6-[4-(methylsulfonyl)tetrahydro-2H-pyran-4-yl]pyrimidin-2-yl}-1H-indole

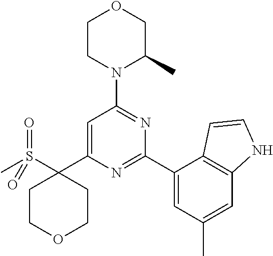

Tris(Dibenzylideneacetone)dipalladium(0) (7.32 mg, 8.00 μmol) and tricyclohexylphosphine (11.96 mg, 0.04 mmol) were added to 4-bromo-6-methyl-1H-indole (112 mg, 0.53 mmol), potassium acetate (78 mg, 0.80 mmol) and bis(pinacolato)diboron (149 mg, 0.59 mmol) in dioxane (8 mL) under nitrogen. The resulting suspension was stirred at 90° C. for 6 hours. (R)-4-(2-Chloro-6-(4-(methylsulfonyl)tetrahydro-2H-pyran-4-yl)pyrimidin-4-yl)-3-methylmorpholine (200 mg, 0.53 mmol), tetrakis(triphenylphosphine)palladium(0) (30.8 mg, 0.03 mmol) and sodium carbonate (2M aqueous solution) (1.066 mL, 2.13 mmol) were added and the resulting suspension was stirred at 90° C. for 18 hours. The reaction mixture was filtered through a whatman 0.45 um PTFE filter and a pl thiol mp spe cartridge. The crude product was purified by preparative HPLC using decreasingly polar mixtures of water (containing 1% NH3) and MeCN as eluents. Fractions containing the desired compound were combined and evaporated to afford the title compound (65 mg, 26%); ¹H NMR (400 MHz, DMSO-d₆) δ 1.27 (3H, d), 2.22-2.29 (2H, m), 2.50 (3H, s), 2.87 (3H, s), 2.90 (2H, s), 3.26-3.28 (3H, m), 3.30 (1H, s), 3.52-3.59 (1H, m), 3.69-3.72 (1H, m), 3.81 (1H, d), 3.93-3.99 (2H, m), 4.01-4.05 (1H, m), 4.31 (1H, d), 4.62-4.64 (1H, m), 6.92 (1H, s), 7.18 (1H, t), 7.34 (1H, s), 7.37 (1H, t), 7.91-7.91 (1H, m), 11.12 (1H, s); m/z: (ESI+) MH⁺, 471.20.

### EXAMPLE 3.10

### 2-Methyl-4-{4-[(3R)-3-methylmorpholin-4-yl]-6-[4-(methylsulfonyl)tetrahydro-2H-pyran-4-yl]pyrimidin-2-yl}-1H-indole

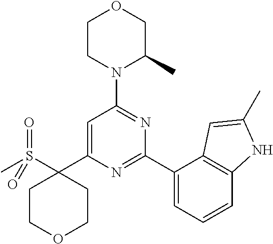

1,1′-Bis(diphenylphosphino)ferrocene-palladium dichloride (11.78 mg, 0.01 mmol) was added to a degassed solution of 2-methyl-1H-indol-4-yl trifluoromethanesulfonate (80 mg, 0.29 mmol), bis(pinacolato)diboron (76 mg, 0.30 mmol), 1,1′-bis(diphenylphosphino)ferrocene (8.03 mg, 0.01 mmol) and potassium acetate (84 mg, 0.86 mmol) in dioxane (5 mL) under nitrogen and the resulting mixture was stirred at 90° C. for 5 hours. (R)-4-(2-Chloro-6-(4-(methylsulfonyl)tetrahydro-2H-pyran-4-yl)pyrimidin-4-yl)-3-methylmorpholine (108 mg, 0.29 mmol), tetrakis(triphenylphosphine)palladium(0) (16.55 mg, 0.01 mmol) and sodium carbonate (2M aqueous solution) (1.000 mL, 2 mmol) were added and heating continued for 24 hours. The mixture was filtered and then evaporated. The residue was purified by preparative HPLC using decreasingly polar mixtures of water (containing 1% NH3) and MeCN as eluents. Fractions containing the desired compound were combined and evaporated to afford the title compound (40 mg, 30%); ¹H NMR (400 MHz, CDCl₃) 1.40 (3H, d), 2.52 (3H, s), 2.54-2.62 (2H, m), 2.71 (3H, s), 2.79 (2H, d), 3.38-3.52 (3H, m), 3.63-3.69 (1H, m), 3.78-3.88 (2H, m), 4.05-4.11 (3H, m), 4.20-4.26 (1H, m), 4.53-4.55 (1H, m), 6.68 (1H, s), 7.06 (1H, s), 7.19 (1H, t), 7.42 (1H, d), 8.04 (1H, s), 8.14-8.16 (1H, m); m/z: (ESI+) MH⁺, 471.

### EXAMPLE 3.11

### 6-Methoxy-4-{4-[(3R)-3-methylmorpholin-4-yl]-6-[4-(methylsulfonyl)tetrahydro-2H-pyran-4-yl]pyrimidin-2-yl}-1H-indole

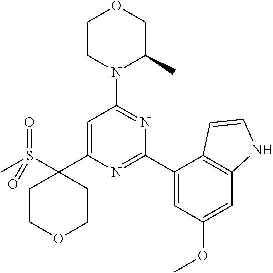

1,1′-Bis(diphenylphosphino)ferrocenedichloro palladium(II) dichloromethane complex (40.6 mg, 0.06 mmol) was added to a degassed solution of 4-bromo-6-methoxy-1H-indole (84 mg, 0.37 mmol), potassium acetate (54.5 mg, 0.56 mmol) and bis(pinacolato)diboron (103 mg, 0.41 mmol) in dioxane (5 mL) under nitrogen. The resulting suspension was stirred at 90° C. for 3 hours. (R)-4-(2-Chloro-6-(4-(methylsulfonyl)tetrahydro-2H-pyran-4-yl)pyrimidin-4-yl)-3-methylmorpholine (139 mg, 0.37 mmol), tetrakis(triphenylphosphine)palladium(0) (21.38 mg, 0.02 mmol) and sodium carbonate (2M aqueous solution) (0.740 mL, 1.48 mmol) were added and the resulting suspension was stirred at 90° C. for 18 hours. The reaction mixture was filtered through a whatman 0.45 um PTFE filter and a pl thiol mp spe cartridge. The crude product was purified by preparative HPLC using decreasingly polar mixtures of water (containing 1% NH3) and MeCN as eluents. Fractions containing the desired compound were combined and evaporated to afford the title compound (26 mg, 14%); ¹H NMR (400 MHz, CDCl₃) 1.40 (3H, d), 2.54-2.62 (2H, m), 2.72 (3H, s), 2.79 (2H, d), 3.37-3.52 (3H, m), 3.62-3.68 (1H, m), 3.78-3.88 (2H, m), 3.91 (3H, s), 4.04-4.11 (3H, m), 4.22 (1H, d), 4.51-4.55 (1H, m), 6.70 (1H, s), 7.05-7.06 (1H, m), 7.23-7.24 (1H, m), 7.28-7.30 (1H, m), 7.89 (1H, d), 8.23 (1H, s); m/z: (ESI+) MH⁺, 487.20.

The following compounds were prepared in a similar way to the method described for Example 3.11, using (R)-4-(2-chloro-6-(4-(methylsulfonyl)tetrahydro-2H-pyran-4-yl)pyrimidin-4-yl)-3-methylmorpholine and the appropriate substituted 4-bromoindole.

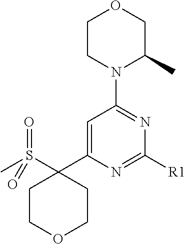

| | | | | m/z |
|---|---|---|---|---|
| Ex. | | | | (ESI+) |
| No. | R1 | Name | ¹H NMR | MH⁺ |
| 3.12 | 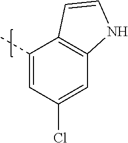 | 6-chloro-4-{4-[(3R)-3- methylmorpholin-4-yl]-6-[4-[4 (methylsulfonyl)tetrahydro-2H- pyran-4-yl]pyrimidin-2-yl}-1H- indole | 1.41 (3H, d), 2.54-2.60 (2H, m), 2.71 (3H, s), 2.77 (2H, d), 3.36-3.50 (3H, m), 3.61 (1H, t), 3.75-3.89 (2H, m), 3.99- 4.10 (3H, m), 4.22 (1H, d), 4.49-4.53 (1H, m), 6.71 (1H, s), 7.26 (1H, s), 7.34-7.36 (2H, m), 7.50 (1H, s), 8.13 (1H, s), 8.31 (1H, s) | 491.15 |
| 3.13 | 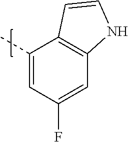 | 6-fluoro-4-{4-[(3R)-3- methylmorpholin-4-yl]-6-[4- (methylsulfonyl)tetrahydro-2H- pyran-4-yl]pyrimidin-2-yl}-1H- indole | 1.40 (3H, s), 2.55-2.64 (2H, m), 2.72 (3H, s), 2.78 (2H, d), 3.38-3.51 (3H, m), 3.62-3.69 (1H, m), 3.78-3.82 (1H, m), 3.88 (1H, d), 4.05-4.12 (3H, m), 4.21 (1H, d), 4.50 (1H, s), 6.71 (1H, s), 7.22-7.25 (1H, m), 7.32-7.37 (2H, m), 7.95- 7.99 (1H, m), 8.32 (1H, s) | 475.19 |
| 3.14 | 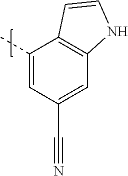 | 4-{4-[(3R)-3-methylmorphohn-4- yl]-6-[4- (methylsulfonyl)tetrahydro-2H- pyran-4-yl]pyrimidin-2-yl}-1H- indole-6-carbonitrile | 1.41 (3H, d), 2.57-2.64 (2H, m), 2.72 (3H, s), 2.74-2.79 (2H, m), 3.39-3.50 (3H, m), 3.63-3.71 (1H, m), 3.79-3.82 (1H, m), 3.89 (1H, d), 4.07- 4.14 (3H, m), 4.21 (1H, d), 4.50 (1H, s), 6.75 (1H, s), 7.45-7.50 (1H, m), 7.52-7.59 (1H, m), 7.84 (1H, s), 8.44 (1H, d), 8.78 (1H, s) | 482.19 |

### EXAMPLE 3.15

### 4-{4-[(3R)-3-Methylmorpholin-4-yl]-6-[4-(methylsulfonyl)piperidin-4-yl]pyrimidin-2-yl}-1H-indole

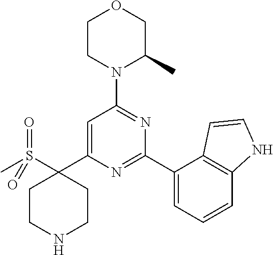

1H-Indol-4-ylboronic acid (55.9 mg, 0.35 mmol), (R)-tert-butyl 4-(2-chloro-6-(3-methylmorpholino)pyrimidin-4-yl)-4-(methylsulfonyl)piperidine-1-carboxylate (150 mg, 0.32 mmol), 2M aqueous sodium carbonate (125 μl, 0.25 mmol) and dichlorobis(triphenylphosphine)palladium(11) (2.217 mg, 3.16 μmol) were suspended in DME:water 4:1 (3 mL) and sealed into a microwave tube. The mixture was heated to 110° C. for 1 hour in a microwave reactor and then cooled to RT. The mixture was applied to a SCX2 column and eluted first with MeOH (2×25 mL) followed by 10% 7N ammonia in MeOH/90% MeOH (2×25 mL). Fractions containing product were combined and evaporated. The residue was dissolved in MeOH (5 mL) and 4N HCl in dioxane (1.5 mL) added. The mixture was stirred at RT overnight. The mixture was evaporated and the residue dissolved in a mixture of MeOH (3 mL) and DMF (0.8 mL) and the pH adjusted to ˜pH 8 with 7N NH3 in MeOH. The mixture was purified by preparative HPLC using decreasingly polar mixtures of water (containing 1% NH3) and MeCN as eluents. Fractions containing the desired compound were combined and evaporated to afford the title compound (34 mg, 23%); ¹H NMR (400 MHz, DMSO-d₆) 1.27 (3H, d), 2.04-2.12 (2H, m), 2.41 (1H, t), 2.82 (3H, s), 2.82-2.89 (2H, m), 2.94-3.04 (2H, m), 3.28 (1H, td), 3.55 (1H, td), 3.71 (1H, dd), 3.81 (1H, d), 4.03 (1H, dd), 4.28-4.31 (1H, m), 4.57-4.67 (1H, m), 6.87 (1H, s), 6.87 (1H, s), 7.20 (1H, t), 7.28-7.31 (1H, m), 7.47 (1H, t), 7.55 (1H, d), 8.11 (1H, d), 11.29 (1H, s); m/z: (ESI+) MH⁺, 456.61.

### EXAMPLE 3.16

### 4-{4-[(3R)-3-Methylmorpholin-4-yl]-6-[1-(methylsulfonyl)cyclobutyl]pyrimidin-2-yl}-1H-indole

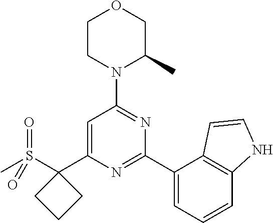

Dichlorobis(triphenylphosphine)palladium(II) (0.812 mg, 1.16 μmol) was added in one portion to (R)-4-(2-chloro-6-(1-(methylsulfonyl)cyclobutyl)pyrimidin-4-yl)-3-methylmorpholine (40 mg, 0.12 mmol), 2M aqueous sodium carbonate solution (0.069 mL, 0.14 mmol) and 1H-indol-4-ylboronic acid (20.48 mg, 0.13 mmol) in DME:water 4:1 (10 mL) at 22° C. and sealed into a microwave tube. The reaction was heated to 110° C. for 1 hour in a microwave reactor and then cooled to RT. The crude product was purified by preparative HPLC using decreasingly polar mixtures of water (containing 1% NH3) and MeCN as eluents. Fractions containing the desired compound were combined and evaporated to afford the title compound (25 mg, 51%); ¹H NMR (400 MHz, CDCl₃) 1.39 (3H, d), 2.02-2.05 (1H, m), 2.30-2.33 (1H, m), 2.76 (3H, s), 2.91-2.97 (2H, m), 3.12-3.19 (2H, m), 3.36-3.43 (1H, m), 3.60-3.67 (1H, m), 3.76-3.85 (2H, m), 4.05 (1H, d), 4.21 (1H, d), 4.56 (1H, d), 6.60 (1H, s), 7.29 (1H, t), 7.33 (1H, t), 7.49-7.51 (1H, m), 7.50-7.54 (1H, m), 8.25-8.27 (1H, m), 8.29 (1H, s); m/z: (ESI+) MH⁺, 427.19.

The (R)-4-(2-chloro-6-(1-(methylsulfonyl)cyclobutyl)pyrimidin-4-yl)-3-methylmorpholine, used as starting material, was prepared as follows:

1,3-Dibromopropane (0.267 mL, 2.62 mmol) was added dropwise over 10 minutes to (R)-4-(2-chloro-6-(methylsulfonylmethyl)pyrimidin-4-yl)-3-methylmorpholine (0.4 g, 1.31 mmol), tetrabutylammonium bromide (0.042 g, 0.13 mmol) and 50% aqueous NaOH (0.314 mL, 3.92 mmol) in toluene (43 mL). The reaction mixture was stirred at RT for 4 hours. The toluene was removed under reduced pressure and the residue dissolved in DCM and the solution washed with water. The organic layer was dried over MgSO4, filtered and then evaporated. The residue was purified by preparative HPLC using decreasingly polar mixtures of water (containing 0.1% NH3) and MeCN as eluents. Fractions containing the desired compound were combined and evaporated to afford (R)-4-(2-chloro-6-(1-(methylsulfonyl)cyclobutyl)pyrimidin-4-yl)-3-methylmorpholine (0.066 g, 15%); m/z: (ESI+) MH⁺, 346.08.

### EXAMPLE 3.17

### 4-{4-[1-(Cyclopropylsulfonyl)cyclopropyl]-6-[(3R)-3-methylmorpholin-4-yl]pyrimidin-2-yl}-1H-indole

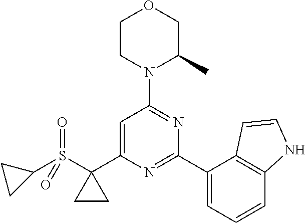

Dichlorobis(triphenylphosphine)palladium(II) (4.96 mg, 7.07 μmol) was added in one portion to (R)-4-(2-chloro-6-(1-(cyclopropylsulfonyl)cyclopropyl)pyrimidin-4-yl)-3-methylmorpholine (253 mg, 0.71 mmol), 2M aqueous sodium carbonate solution (0.424 mL, 0.85 mmol) and 1H-indol-4-ylboronic acid (137 mg, 0.85 mmol) in DME:water 4:1 (5 mL) at RT. The mixture was heated to 90° C. for 1 hour and then allowed to cool to RT. The mixture was diluted with EtOAc (50 mL) and then washed sequentially with water (50 mL) and saturated brine (50 mL). The organic layer was separated and then evaporated onto silica. The residue was purified by chromatography on silica with an elution gradient of 0 to 100% EtOAc in isohexane. Pure fractions were combined and evaporated. The residue was purified by preparative HPLC using decreasingly polar mixtures of water (containing 1% NH3) and MeCN as eluents. Fractions containing the desired compound were combined and evaporated to afford the title compound (118 mg, 38%); ¹H NMR (400 MHz, DMSO-d₆) 0.93-1.03 (4H, m), 1.28 (3H, d), 1.61-1.66 (2H, m), 1.68-1.72 (2H, m), 2.98-3.04 (1H, m), 3.23-3.32 (1H, m) partially obscured by water signal, 3.49-3.56 (1H, m), 3.66-3.69 (1H, m), 3.80 (1H, d), 3.99-4.03 (1H, m), 4.19 (1H, d), 4.58 (1H, d), 6.92 (1H, s), 7.19 (1H, t), 7.37 (1H, t), 7.45 (1H, t), 7.54 (1H, d), 8.07-8.10 (1H, m), 11.22 (1H, s); m/z: (ESI+) MH⁺, 439.57.

The (R)-4-(2-chloro-6-(1-(cyclopropylsulfonyl)cyclopropyl)pyrimidin-4-yl)-3-methylmorpholine, used as starting material, was prepared as follows:

a) Sodium cyclopropanesulfinate (1.389 g, 10.84 mmol) was added in one portion to (R)-4-(2-chloro-6-(iodomethyl)pyrimidin-4-yl)-3-methylmorpholine (3 g, 8.48 mmol) in MeCN (85 mL) at RT. The resulting mixture was stirred under nitrogen at 80° C. for 18 hours. The reaction mixture was evaporated and the residue dissolved in DCM (125 mL). The solution was washed sequentially with water (125 mL), 2M sodium bisulfite solution (125 mL) and saturated brine (125 mL). The organic layer was dried over MgSO4, filtered and then evaporated. The residue was triturated with diethyl Et2O and the mixture filtered. The solid was washed with Et2O (5 mL) and then air dried to afford (R)-4-(2-chloro-6-(cyclopropylsulfonylmethyl)pyrimidin-4-yl)-3-methylmorpholine (2.350 g, 83%) which was used in the next step without further purification; ¹H NMR (400 MHz, DMSO-d₆) 0.95-0.99 (2H, m), 1.02-1.08 (2H, m), 1.23 (3H, d), 2.78-2.84 (1H, m), 3.19-3.26 (1H, m), 3.43-3.49 (1H, m), 3.59-3.63 (1H, m), 3.74 (1H, d), 3.93-3.97 (2H, m), 4.31 (1H, br s), 4.49 (2H, s), 6.93 (1H, s); m/z: (ESI+) MH⁺, 332.42.

b) A 50% aqueous solution of NaOH (2 mL) was added to (R)-4-(2-chloro-6-(cyclopropylsulfonylmethyl)pyrimidin-4-yl)-3-methylmorpholine (650 mg, 1.96 mmol), 1,2-dibromoethane (0.169 mL, 1.96 mmol) and tetrabutylammonium bromide (63.1 mg, 0.20 mmol) in toluene (6 mL). The resulting slurry was stirred at 60° C. for 1 hour. EtOAc (100 mL) was added and the mixture washed with water (100 mL) and brine (100 mL). The mixture was dried over MgSO₄ and evaporated onto silica. The residue was purified by chromatography on silica with an elution gradient of 30 to 60% EtOAc in DCM. Pure fractions were combined and evaporated to afford (R)-4-(2-chloro-6-(1-(cyclopropylsulfonyl)cyclopropyl)pyrimidin-4-yl)-3-methylmorpholine (545 mg, 78%); ¹H NMR (400 MHz, DMSO-d₆) δ 0.89-0.92 (2H, m), 1.00-1.05 (2H, m), 1.21 (3H, d), 1.50-1.53 (2H, m), 1.61-1.64 (2H, m), 2.90-2.96 (1H, m), 3.20-3.24 (1H, m), 3.40-3.47 (1H, m), 3.56-3.60 (1H, m), 3.72 (1H, d), 3.92-3.95 (1H, m), 4.03 (1H, br s), 4.39 (1H, br s), 6.99 (1H, s); m/z: (ESI+) MH⁺, 358.43.

### EXAMPLE 3.18

### 4-{4-[4-(Cyclopropylsulfonyl)piperidin-4-yl]-6-[(3R)-3-methylmorpholin-4-yl]pyrimidin-2-yl}-1H-indole

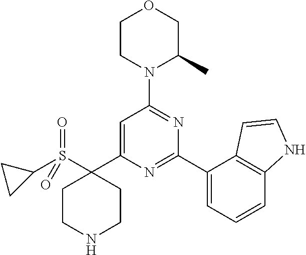

Dichlorobis(triphenylphosphine)palladium(II) (3.64 mg, 5.19 μmol) was added in one portion to (R)-tert-butyl 4-(2-chloro-6-(3-methylmorpholino)pyrimidin-4-yl)-4-(cyclopropylsulfonyl)piperidine-1-carboxylate (260 mg, 0.52 mmol), 2M aqueous sodium carbonate solution (0.311 mL, 0.62 mmol) and 1H-indol-4-ylboronic acid (100 mg, 0.62 mmol) in DME:water 4:1 (5 mL) at RT. The mixture was heated to 90° C. for 1 hour and then loaded onto an SCX column. The column was eluted first with MeOH and then with 7N NH3/MeOH. Fractions containing the desired product were combined and evaporated. The residue was dissolved in DCM (50 mL), washed with water (50 mL) and the organic layer concentrated under reduced pressure. The residue was treated with 10% TFA in DCM (5 mL) and stirred at RT for 1 hour. The crude product was purified by ion exchange chromatography, using an SCX column. The desired product was eluted from the column using 7M NH3/MeOH and then evaporated onto silica. The residue was purified by chromatography on silica with an elution gradient of 0 to 5% 7M NH3/MeOH in DCM. Pure fractions were combined and evaporated to afford the title compound (157 mg, 63%); ¹H NMR (500 MHz, DMSO-d₆) 0.74-0.77 (2H, m), 0.85 (2H, d), 1.27 (3H, d), 2.09-2.14 (2H, m), 2.42-2.52 (2H, m), 2.95-2.99 (4H, m), 3.24-3.27 (1H, m), 3.54-3.58 (1H, m), 3.69-3.72 (1H, m), 3.81 (1H, d), 4.01-4.04 (1H, m), 4.27 (1H, d), 4.61 (1H, s), 6.89 (1H, s), 7.20 (1H, t), 7.34 (1H, s), 7.46 (1H, t), 7.54 (1H, d), 8.13 (1H, d), 11.24 (1H, s); m/z: (ESI+) MH⁺, 482.58.

The (R)-tert-butyl 4-(2-chloro-6-(3-methylmorpholino)pyrimidin-4-yl)-4-(cyclopropylsulfonyl)piperidine-1-carboxylate, used as starting material, was prepared as follows:

a) A solution of (R)-4-(2-chloro-6-(cyclopropylsulfonylmethyl)pyrimidin-4-yl)-3-methylmorpholine (865 mg, 2.61 mmol) in NMP (10 mL) was treated with sodium hydride, 60% dispersion in mineral oil (344 mg, 8.60 mmol). The mixture was stirred at RT for 10 minutes before being treated with tetrabutylammonium bromide (1261 mg, 3.91 mmol) and N-benzyl-2-chloro-N-(2-chloroethyl)ethanamine hydrochloride (770 mg, 2.87 mmol). The reaction mixture was stirred for 5 minutes at RT and then heated to 50° C. for 1 hour and then at 80° C. for 1 hour; the mixture was then allowed to stand at RT overnight. The mixture was quenched by the addition of saturated ammonium chloride solution and then extracted with EtOAc (3×50 mL). The organic solution was washed three times with water (100 mL), saturated brine (100 mL), dried over magnesium sulfate, filtered, and then concentrated onto silica. The residue was purified by chromatography on silica eluting with a gradient of 10 to 70% EtOAc in DCM. Pure fractions were combined and evaporated to afford (R)-4-(6-(1-benzyl-4-(cyclopropylsulfonyl)piperidin-4-yl)-2-chloropyrimidin-4-yl)-3-methylmorpholine (976 mg, 76%); ¹H NMR (400 MHz, DMSO-d₆) 0.70-0.77 (2H, m), 0.90-0.96 (2H, m), 1.18-1.21 (3H, m), 1.78-1.88 (2H, m), 2.09-2.16 (2H, m), 2.55 (1H, m, partially obscured by DMSO peak), 2.78-2.84 (4H, m), 3.17-3.23 (1H, m), 3.36 (2H, s), 3.43-3.50 (1H, m), 3.60-3.63 (1H, m), 3.73 (1H, d), 3.92-3.96 (1H, m), 4.07-4.13 (1H, m), 4.43 (1H, br s), 6.96 (1H, s), 7.23-7.34 (5H, m); m/z: (ESI+) MH⁺, 491.51.

b) 1-Chloroethyl chloroformate (0.426 mL, 3.95 mmol) was added to a solution of (R)-4-(6-(1-benzyl-4-(cyclopropylsulfonyl)piperidin-4-yl)-2-chloropyrimidin-4-yl)-3-methylmorpholine (970 mg, 1.98 mmol) in DCM (10 mL). The solution was heated at reflux for 3 hours and then allowed to cool to RT. The mixture was diluted with MeOH (12 mL) and allowed to stand overnight. The mixture was treated with di-tert-butyl dicarbonate (948 mg, 4.35 mmol) and N-ethyldiisopropylamine (0.684 mL, 3.95 mmol) and the solution stirred at RT for 3 hours. The solution was partitioned between DCM and water, the organic phase separated and then dried over MgSO4 and concentrated under reduced pressure onto silica. The residue was purified by chromatography on silica eluting with a gradient of 10 to 30% EtOAc in DCM. Pure fractions were combined and evaporated to afford (R)-tert-butyl 4-(2-chloro-6-(3-methylmorpholino)pyrimidin-4-yl)-4-(cyclopropylsulfonyl)piperidine-1-carboxylate (629 mg, 64%); ¹H NMR (400 MHz, DMSO-d₆) 0.72-0.79 (2H, m), 0.91-0.96 (2H, m), 1.21 (3H, d), 1.40 (9H, s), 1.92-2.00 (2H, m), 2.53-2.61 (1H, m), 2.84 (2H, m), 3.17-3.23 (1H, m), 3.42-3.49 (1H, m), 3.59-3.63 (1H, m), 3.73 (1H, d), 3.93-3.97 (3H, m), 4.11 (1H, br d), 4.48 (1H, br s), 6.97 (1H, s); m/z: (ESI+) MH⁺, 501.50.

### EXAMPLE 3.19

### 4-{4-[4-(Cyclopropylsulfonyl)tetrahydro-2H-pyran-4-yl]-6-[(3R)-3-methylmorpholin-4-yl]pyrimidin-2-yl}-1H-indole

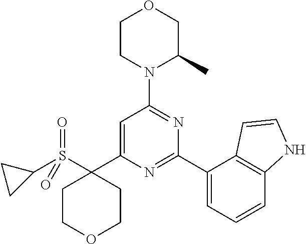

Dichlorobis(triphenylphosphine)palladium(II) (4.03 mg, 5.75 μmol) was added in one portion to (R)-4-(2-chloro-6-(4-(cyclopropylsulfonyl)tetrahydro-2H-pyran-4-yl)pyrimidin-4-yl)-3-methylmorpholine (0.231 g, 0.57 mmol), 2M aqueous sodium carbonate solution (0.345 mL, 0.69 mmol) and 1H-indol-4-ylboronic acid (0.111 g, 0.69 mmol) in DME:water 4:1 (5 mL) at RT. The mixture was heated to 90° C. for 30 minutes and then allowed to cool to RT. The mixture was diluted with EtOAc (50 mL) and washed sequentially with water (50 mL) and a saturated brine solution (50 mL). The organic layer was evaporated onto silica and the residue was purified by chromatography on silica eluting with a gradient of 0 to 100% EtOAc in isohexane. Pure fractions were combined and evaporated. The residue was purified by preparative HPLC using decreasingly polar mixtures of water (containing 1% NH3) and MeCN as eluents. Fractions containing the desired compound were combined and evaporated to afford the title compound (0.108 g, 39%); ¹H NMR (400 MHz, DMSO-d₆) 0.77-0.83 (2H, m), 0.84-0.88 (2H, m), 1.27 (3H, d), 2.25-2.33 (2H, m), 2.94-2.99 (2H, m), 3.24-3.27 (2H, m) partially obscured by water, 3.52-3.59 (1H, m), 3.69-3.72 (1H, m), 3.81 (1H, d), 3.94 (2H, t), 4.00-4.04 (1H, m), 4.28 (1H, d), 4.63 (1H, d), 6.94 (1H, s), 7.20 (1H, t), 7.31 (1H, t), 7.45 (1H, t), 7.54 (1H, d), 8.11-8.13 (1H, m), 11.23 (1H, s); m/z: (ESI+) MH⁺, 483.57.

The (R)-4-(2-chloro-6-(4-(cyclopropylsulfonyl)tetrahydro-2H-pyran-4-yl)pyrimidin-4-yl)-3-methylmorpholine, used as starting material, was prepared as follows:

A 50% aqueous NaOH solution (2 mL) was added to (R)-4-(2-chloro-6-(cyclopropylsulfonylmethyl)pyrimidin-4-yl)-3-methylmorpholine, tetrabutylammonium bromide (63.1 mg, 0.20 mmol) and 1-bromo-2-(2-bromoethoxy)ethane (0.244 mL, 1.96 mmol) in toluene (6 mL) at RT. The resulting mixture was stirred under nitrogen at 60° C. for 3 hours. The mixture was diluted with EtOAc (100 mL) and washed sequentially with water (75 mL) and saturated brine (75 mL). The organic layer was dried over MgSO4, filtered and evaporated. The residue was triturated with MeOH and the formed solid collected by filtration, washed with MeOH (10 mL) and then air dried to afford (R)-4-(2-chloro-6-(4-(cyclopropylsulfonyl)tetrahydro-2H-pyran-4-yl)pyrimidin-4-yl)-3-methylmorpholine (296 mg, 37.6%) which was used in the next step without further purification; ¹H NMR (400 MHz, DMSO-d₆) 0.71-0.80 (2H, m), 0.93-0.98 (2H, m), 1.20 (3H, d), 2.12-2.20 (2H, m), 2.50-2.58 (1H, m) partially obscured by DMSO peak, 2.71-2.76 (3H, m), 3.15-3.23 (2H, m), 3.43-3.50 (1H, m), 3.59-3.63 (1H, m), 3.73 (1H, d), 3.86-3.91 (2H, m), 3.92-3.96 (1H, m), 4.12 (1H, br s), 4.46 (1H, br s), 7.00 (1H, s); m/z: (ESI+) MH⁺, 402.45.

The mother liquors were evaporated onto silica and the residue purified by chromatography on silica eluting with a gradient of 20 to 60% EtOAc in DCM. Pure fractions were combined and evaporated to afford a second sample of (R)-4-(2-chloro-6-(4-(cyclopropylsulfonyl)tetrahydro-2H-pyran-4-yl)pyrimidin-4-yl)-3-methylmorpholine (275 mg, 35%); ¹H NMR (400 MHz, DMSO-d₆) δ 0.71-0.79 (2H, m), 0.90-0.99 (2H, m), 1.20 (3H, d), 2.12-2.20 (2H, m), 2.50-2.57 (1H, m, partially obscured by DMSO), 2.69-2.76 (2H, m), 3.15-3.23 (3H, m), 3.43-3.50 (1H, m), 3.59-3.63 (1H, m), 3.73 (1H, d), 3.86-3.91 (2H, m), 3.92-3.96 (1H, m), 4.11 (1H, q), 4.46 (1H, s), 7.00 (1H, s); m/z: (ESI+) MH⁺, 402.42.

### EXAMPLE 3.20

### 4-{4-[1-(Ethylsulfonyl)cyclopropyl]-6-[(3R)-3-methylmorpholin-4-yl]pyrimidin-2-yl}-1H-indole

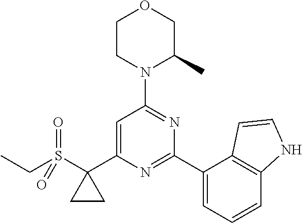

Dichlorobis(triphenylphosphine)palladium(II) (5.07 mg, 7.23 μmol) was added in one portion to (R)-4-(2-chloro-6-(1-(ethylsulfonyl)cyclopropyl)pyrimidin-4-yl)-3-methylmorpholine (250 mg, 0.72 mmol), 2M aqueous sodium carbonate solution (0.434 mL, 0.87 mmol) and 1H-indol-4-ylboronic acid (128 mg, 0.80 mmol) in DME:water 4:1 (10 mL) at 22° C. and sealed into a microwave tube. The mixture was heated to 110° C. for 1.5 hours in a microwave reactor and then allowed to cool to RT. Dichlorobis(triphenylphosphine)palladium(II) (5.07 mg, 7.23 μmol) was added and the mixture heated to 110° C. in a microwave reactor for a further 30 minutes. The mixture was purified by ion exchange chromatography, using an SCX column. The desired product was eluted from the column using 7M NH3/MeOH. Fractions containing product were combined and evaporated onto silica. The residue was purified by chromatography on silica eluting with a gradient of 0 to 50% EtOAc in isohexane. Pure fractions were combined and evaporated to afford the title compound (170 mg, 55%); ¹H NMR (400 MHz, DMSO-d₆) 1.27-1.32 (6H, m), 1.60-1.64 (2H, m), 1.67-1.71 (2H, m), 3.23-3.33 (1H, m, partially obscured by water), 3.44 (2H, q), 3.49-3.56 (1H, m), 3.65-3.69 (1H, m), 3.81 (1H, d), 3.99-4.03 (1H, m), 4.21 (1H, d), 4.60 (1H, d), 6.85 (1H, s), 7.21 (1H, t), 7.31 (1H, d), 7.46 (1H, t), 7.55 (1H, d), 8.04-8.06 (1H, m), 11.25 (1H, s); m/z: (ESI+) MH⁺, 427.52.

The (R)-4-(2-chloro-6-(1-(ethylsulfonyl)cyclopropyl)pyrimidin-4-yl)-3-methylmorpholine, used as starting material, was prepared as follows:

a) Sodium ethanesulfinate (0.854 g, 7.35 mmol) was added in one portion to (R)-4-(2-chloro-6-(iodomethyl)pyrimidin-4-yl)-3-methylmorpholine (2 g, 5.66 mmol) in MeCN (56.6 mL) at RT. The resulting mixture was stirred under nitrogen at 80° C. for 18 hours. The mixture was evaporated and the residue dissolved in DCM (250 mL) and washed sequentially with water (250 mL), 2M sodium bisulfite solution (250 mL), and saturated brine (250 mL). The organic layer was dried over MgSO4, filtered and then evaporated. The residue was triturated with Et2O to afford a precipitate. The precipitate was collected by filtration, washed with Et₂O (5 mL) and then air dried to afford (R)-4-(2-chloro-6-(ethylsulfonylmethyl)pyrimidin-4-yl)-3-methylmorpholine (1.520 g, 84%) which was used in the next step without further purification; ¹H NMR (400 MHz, CDCl₃) 1.34 (3H, d), 1.44 (3H, t), 3.14 (2H, q), 3.27-3.34 (1H, m), 3.51-3.57 (1H, m), 3.67-3.70 (1H, m), 3.79 (1H, d), 3.95-4.11 (1H, m), 3.99-4.03 (1H, m), 4.15 (2H, s), 4.31 (1H, br s), 6.53 (1H, s); m/z: (ESI+) MH⁺, 320.41.

b) A 50% aqueous solution of NaOH (2 mL) was added to (R)-4-(2-chloro-6-(ethylsulfonylmethyl)pyrimidin-4-yl)-3-methylmorpholine (645 mg, 2.02 mmol), 1,2-dibromoethane (0.174 mL, 2.02 mmol) and tetrabutylammonium bromide (65.0 mg, 0.20 mmol) in toluene (6 mL). The resulting slurry was stirred at 60° C. for 3 hours. EtOAc (200 mL) as added and the mixture washed with water (100 mL) and brine (100 mL). The organic solution was dried over MgSO₄ and then concentrated in vacuo. The residue was triturated with MeOH and then dried under vacuum to afford (R)-4-(2-chloro-6-(1-(ethylsulfonyl)cyclopropyl)pyrimidin-4-yl)-3-methylmorpholine (395 mg. 57%) which was used in the next step without further purification; ¹H NMR (400 MHz, CDCl₃) 1.33 (3H, d), 1.38 (3H, t), 1.48 (2H, m), 1.78-1.81 (2H, m), 3.16 (2H, q), 3.26-3.33 (1H, m), 3.50-3.57 (1H, m), 3.66-3.70 (1H, m), 3.78 (1H, d), 3.98-4.02 (2H, m), 4.33 (1H, s), 6.86 (1H, s); m/z: (ESI+) MH⁺, 346.44.

The mother liquors were evaporated and the residue triturated with MeOH to afford a second crop of solid which was dried under vacuum to afford (R)-4-(2-chloro-6-(1-(ethylsulfonyl)cyclopropyl)pyrimidin-4-yl)-3-methylmorpholine (140 mg, 20%) which was used in the next step without further purification; ¹H NMR (400 MHz, CDCl₃) 1.33 (3H, d), 1.38 (3H, t), 1.48 (2H, m), 1.78-1.81 (2H, m), 3.16 (2H, q), 3.26-3.33 (1H, m), 3.50-3.57 (1H, m), 3.66-3.70 (1H, m), 3.78 (1H, d), 3.98-4.02 (2H, m), 4.33 (1H, s), 6.86 (1H, s); m/z: (ESI+) MH⁺, 346.44.

### EXAMPLE 3.21

### 4-{4-[4-(Ethylsulfonyl)tetrahydro-2H-pyran-4-yl]-6-[(3R)-3-methylmorpholin-4-yl]pyrimidin-2-yl}-1H-indole

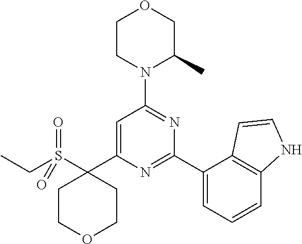

Dichlorobis(triphenylphosphine)palladium(II) (4.86 mg, 6.92 μmol) was added in one portion to (R)-4-(2-chloro-6-(4-(ethylsulfonyl)tetrahydro-2H-pyran-4-yl)pyrimidin-4-yl)-3-methylmorpholine (270 mg, 0.69 mmol), 2M aqueous sodium carbonate solution (0.415 mL, 0.83 mmol) and 1H-indol-4-ylboronic acid (134 mg, 0.83 mmol) in DME:water 4:1 (10 mL) at RT. The mixture was heated to 110° C. for 1 hour. The crude product was purified by ion exchange chromatography, using an SCX column. The desired product was eluted from the column using 7M NH3/MeOH and fractions containing product were combined and evaporated onto silica. The residue was purified by chromatography on silica eluting with a gradient of 0 to 100% EtOAc in isohexane. Pure fractions were combined and evaporated to afford the title compound (190 mg, 58%); ¹H NMR (400 MHz, DMSO-d₆) 1.08 (3H, t), 1.28 (3H, d), 2.25-2.32 (2H, m), 2.88-2.93 (2H, m), 3.00 (2H, q), 3.24-3.32 (3H, m, partially obscured by water peak), 3.53-3.59 (1H, m), 3.69-3.73 (1H, m), 3.81 (1H, d), 3.93-3.99 (2H, m), 4.01-4.05 (1H, m), 4.30 (1H, d), 4.63 (1H, d), 6.93 (1H, s), 7.21 (1H, t), 7.27 (1H, d), 7.47 (1H, t), 7.56 (1H, d), 8.10-8.13 (1H, m), 11.27 (1H, s); m/z: (ESI+) MH⁺, 471.55.

The (R)-4-(2-chloro-6-(4-(ethylsulfonyl)tetrahydro-2H-pyran-4-yl)pyrimidin-4-yl)-3-methylmorpholine, used as starting material, was prepared as follows:

A 50% aqueous solution of NaOH (2 mL) was added to (R)-4-(2-chloro-6-(ethylsulfonylmethyl)pyrimidin-4-yl)-3-methylmorpholine (650 mg, 2.03 mmol), tetrabutylammonium bromide (65.5 mg, 0.20 mmol) and 1-bromo-2-(2-bromoethoxy)ethane (471 mg, 2.03 mmol) in toluene (6 mL) at RT. The resulting mixture was stirred under nitrogen at 60° C. for 2 hours. The mixture was diluted with EtOAc (100 mL), and washed sequentially with water (75 mL), 1M sodium bisulfite solution (75 mL), and saturated brine (75 mL). The organic layer was dried over MgSO4, filtered and then evaporated. The residue was triturated with MeOH and the formed solid was collected by filtration, washed with MeOH (10 mL) and then air dried to afford (R)-4-(2-chloro-6-(4-(ethylsulfonyl)tetrahydro-2H-pyran-4-yl)pyrimidin-4-yl)-3-methylmorpholine (516 mg, 65%) which was used without further purification; ¹H NMR (400 MHz, CDCl₃) 1.29 (3H, t), 1.32 (2H, s), 1.34 (2H, s), 2.46-2.58 (4H, m), 2.86 (2H, q), 3.27-3.38 (3H, m), 3.53-3.59 (1H, m), 3.69-3.72 (1H, m), 3.79 (1H, d), 3.98-4.04 (4H, m), 4.31 (2H, s), 6.64 (1H, s); m/z: (ESI+) MH⁺, 390.45.

### EXAMPLE 3.22

### 4-{4-[4-(Ethylsulfonyl)piperidin-4-yl]-6-[(3R)-3-methylmorpholin-4-yl]pyrimidin-2-yl}-1H-indole

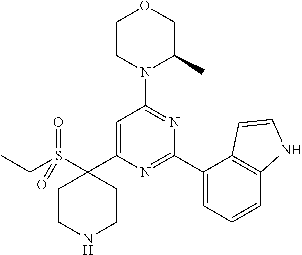

Dichlorobis(triphenylphosphine)palladium(II) (3.73 mg, 5.32 μmol) was added in one portion to (R)-tert-butyl 4-(2-chloro-6-(3-methylmorpholino)pyrimidin-4-yl)-4-(ethylsulfonyl)piperidine-1-carboxylate (260 mg, 0.53 mmol), 2M aqueous sodium carbonate solution (0.319 mL, 0.64 mmol) and 1H-indol-4-ylboronic acid (103 mg, 0.64 mmol) in DME:water 4:1 (5 mL) at RT. The mixture was heated to 90° C. for 1 hour. The reaction mixture was loaded onto an SCX column, and eluted first with MeOH and then with 7N NH3/MeOH. Fractions containing product were combined and evaporated. The residue was dissolved in DCM (50 mL), washed with water (50 mL) and the organic layer concentrated under reduced pressure. The residue was treated with 10% TFA in DCM (5 mL) and stirred at RT for 1 hour. The mixture was purified by ion exchange chromatography, using an SCX column. The desired product was eluted from the column using 7M NH3/MeOH and fractions containing product were combined and evaporated onto silica. The residue was purified by chromatography on silica eluting with a gradient of 0 to 5% 7M NH3/MeOH in DCM. Pure fractions were combined and evaporated to afford the title compound (154 mg, 62%); ¹H NMR (500 MHz, DMSO-d₆) 1.06 (3H, t), 1.27 (3H, d), 2.09-2.13 (2H, m), 2.40-2.46 (2H, m), 2.86 (2H, d), 2.94-3.02 (4H, m), 3.54-3.58 (1H, m), 3.71 (1H, d), 3.81 (1H, s), 4.03 (1H, d), 4.28 (1H, d), 4.60 (1H, s), 6.88 (1H, s), 7.21 (1H, t), 7.30 (1H, s), 7.46 (1H, s), 7.55 (1H, d), 8.11 (1H, d), 11.26 (1H, s); m/z: (ESI+) MH⁺, 470.58.

The (R)-tert-butyl 4-(2-chloro-6-(3-methylmorpholino)pyrimidin-4-yl)-4-(ethylsulfonyl)piperidine-1-carboxylate, used as starting material, was prepared as follows:

a) A solution of (R)-4-(2-chloro-6-(ethylsulfonylmethyl)pyrimidin-4-yl)-3-methylmorpholine (1 g, 3.13 mmol) in NMP (9.5 mL) was treated with sodium hydride, 60% dispersion in mineral oil (0.413 g, 10.32 mmol). The mixture was stirred at RT for 10 minutes before being treated with tetrabutylammonium bromide (1.512 g, 4.69 mmol) and N-benzyl-2-chloro-N-(2-chloroethyl)ethanamine hydrochloride (0.924 g, 3.44 mmol). The mixture was stirred for 5 minutes and then heated to 50° C. for 1 hour and then at 80° C. for 1.5 hours. The mixture was allowed to cool to RT and then quenched by the addition of saturated ammonium chloride solution. The mixture was extracted with EtOAc and the organic solution washed with water (×3) and saturated brine, dried over MgSO4, filtered, and then concentrated under reduced pressure. The residue was purified by chromatography on silica eluting with a gradient of 10 to 70% EtOAc in DCM. Pure fractions were combined and evaporated to afford (R)-4-(6-(1-benzyl-4-(ethylsulfonyl)piperidin-4-yl)-2-chloropyrimidin-4-yl)-3-methylmorpholine (1.280 g, 85%); ¹H NMR (400 MHz, DMSO-d₆) 1.12 (3H, t), 1.21 (3H, d), 1.83 (2H, q), 2.10-2.17 (2H, m), 2.72-2.84 (4H, m), 2.98 (2H, q), 3.19-3.24 (1H, m), 3.37 (2H, s), 3.43-3.50 (1H, m), 3.60-3.64 (1H, m), 3.73 (1H, d), 3.92-3.96 (1H, m), 4.10 (1H, br d), 4.44 (1H, br s), 6.94 (1H, s), 7.22-7.33 (5H, m); m/z: (ESI+) MH⁺, 479.52.

b) 1-Chloroethyl chloroformate (0.60 mL, 5.56 mmol) was added to a solution of (R)-4-(6-(1-benzyl-4-(ethylsulfonyl)piperidin-4-yl)-2-chloropyrimidin-4-yl)-3-methylmorpholine (1.27 g, 2.65 mmol) in DCM (10 mL). The solution was heated at reflux for 3 hours and then allowed to cool to RT. The mixture diluted with MeOH (12 mL) and allowed to stand overnight. The mixture was treated with di-tert-butyl dicarbonate (1.273 g, 5.83 mmol) and N-ethyldiisopropylamine (0.917 mL, 5.30 mmol) and then stirred at RT for 2 hours. Further di-tert-butyl dicarbonate (0.13 g, 0.58 mmol) and N-ethyldiisopropylamine (0.10 mL, 0.50 mmol) were added and the solution stirred at RT for a further 2 hours. The solution was partitioned between DCM and water. The organic phase was separated, dried over MgSO4 and then concentrated under reduced pressure onto silica. The residue was purified by chromatography on silica eluting with a gradient of 10 to 30% EtOAc in DCM. Pure fractions were combined and evaporated to afford (R)-tert-butyl 4-(2-chloro-6-(3-methylmorpholino)pyrimidin-4-yl)-4-(ethylsulfonyl)piperidine-1-carboxylate (1.140 g, 88%); ¹H NMR (400 MHz, DMSO-d₆) 1.13 (3H, t), 1.22 (3H, d), 1.40 (9H, s), 1.92-1.99 (2H, m), 2.62 (2H, br s), 2.75 (2H, d), 3.00 (2H, q), 3.19-3.25 (1H, m), 3.43-3.50 (1H, m), 3.59-3.63 (1H, m), 3.73 (1H, d), 3.93-3.97 (3H, m), 4.12 (1H, d), 4.46 (1H, s), 6.95 (1H, s); m/z: (ESI+) MH⁺, 489.51.

### EXAMPLE 3.23

### 4-(4-{1-[(1-Methylethyl)sulfonyl]cyclopropyl}-6-[(3R)-3-methylmorpholin-4-yl]pyrimidin-2-yl)-1H-indole

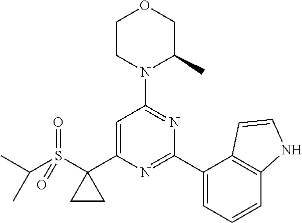

Dichlorobis(triphenylphosphine)palladium(II) (2.477 mg, 3.53 μmol) was added in one portion to (R)-4-(2-chloro-6-(1-(isopropylsulfonyl)cyclopropyl)pyrimidin-4-yl)-3-methylmorpholine (127 mg, 0.35 mmol), 2M aqueous sodium carbonate solution (0.212 mL, 0.42 mmol) and 1H-indol-4-ylboronic acid (62.5 mg, 0.39 mmol) in DME:water 4:1 (10 mL) at 22° C. and sealed into a microwave tube. The reaction was heated to 110° C. for 1 hour in a microwave reactor and then allowed to cool to RT. The mixture was purified by ion exchange chromatography, using an SCX column. The desired product was eluted from the column using 7M NH3/MeOH and pure fractions were combined and evaporated. The residue was purified by preparative HPLC using decreasingly polar mixtures of water (containing 1% NH3) and MeCN as eluents. Fractions containing the desired compound were combined and evaporated to afford the title compound (38.0 mg, 24%); ¹H NMR (400 MHz, DMSO-d₆) 1.27-1.32 (9H, m), 1.60-1.67 (4H, m), 3.23-3.27 (1H, m), 3.49-3.56 (1H, m), 3.62-3.69 (2H, m), 3.80 (1H, d), 3.99-4.03 (1H, m), 4.19 (1H, d), 4.57 (1H, d), 6.87 (1H, s), 7.20 (1H, t), 7.33 (1H, t), 7.46 (1H, t), 7.54 (1H, d), 8.05-8.08 (1H, m), 11.24 (1H, s); m/z: (ESI+) MH⁺, 441.18.

The (R)-4-(2-chloro-6-(1-(isopropylsulfonyl)cyclopropyl)pyrimidin-4-yl)-3-methylmorpholine, used as starting material, was prepared as follows:

a) Sodium propane-2-sulfinate (1.270 g, 9.76 mmol) was added in one portion to (R)-4-(2-chloro-6-(iodomethyl)pyrimidin-4-yl)-3-methylmorpholine (3.45 g, 9.76 mmol) in DMF (70 mL). The resulting mixture was stirred at 25° C. for 18 hours and then diluted with DCM. The mixture was washed with water (2×300 mL), aqueous sodium thiosulphate (200 mL), brine (200 mL), dried over MgSO4 and then concentrated in vacuo. The residue was triturated with MeOH to give a solid which was collected by filtration and dried under vacuum to afford (R)-4-(2-chloro-6-(isopropylsulfonylmethyl)pyrimidin-4-yl)-3-methylmorpholine (2.400 g, 74%); m/z: (ESI+) MH⁺, 334.11.

b) A 50% aqueous solution of NaOH (14 mL) was added to (R)-4-(2-chloro-6-(isopropylsulfonylmethyl)pyrimidin-4-yl)-3-methylmorpholine (550 mg, 1.65 mmol), 1,2-dibromoethane (0.142 mL, 1.65 mmol) and tetrabutylammonium bromide (53.1 mg, 0.16 mmol) in toluene (40 mL). The resulting slurry was stirred at 60° C. for 3 hours. EtOAc (150 mL) was added and the mixture was washed with water (100 mL), brine (100 mL), dried over MgSO₄ and then concentrated in vacuo. The residue was purified by chromatography on silica eluting with a gradient of 10 to 90% EtOAc in isohexane. Pure fractions were combined and evaporated to to afford (R)-4-(2-chloro-6-(1-(isopropylsulfonyl)cyclopropyl)pyrimidin-4-yl)-3-methylmorpholine (425 mg, 72%); m/z: (ESI+) MH⁺, 360.09.

### EXAMPLE 3.24

### 4-(4-{4-[(1-Methylethyl)sulfonyl]tetrahydro-2H-pyran-4-yl}-6-[(3R)-3-methylmorpholin-4-yl]pyrimidin-2-yl)-1H-indole

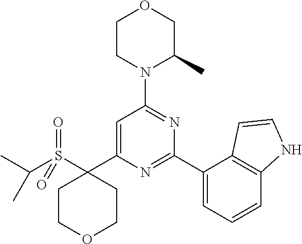

Dichlorobis(triphenylphosphine)palladium(II) (3.70 mg, 5.27 μmol) was added in one portion to (R)-4-(2-chloro-6-(4-(isopropylsulfonyl)tetrahydro-2H-pyran-4-yl)pyrimidin-4-yl)-3-methylmorpholine (213 mg, 0.53 mmol), 2M aqueous sodium carbonate solution (0.316 mL, 0.63 mmol) and 1H-indol-4-ylboronic acid (93 mg, 0.58 mmol) in DME:water 4:1 (10 mL) at 22° C. and sealed into a microwave tube. The reaction was heated to 110° C. for 1 hour in a microwave reactor and then allowed to cool to RT. The mixture was purified by preparative HPLC using decreasingly polar mixtures of water (containing 1% NH3) and MeCN as eluents. Fractions containing the desired compound were combined and evaporated to afford the title compound (131 mg, 51%); ¹H NMR (400 MHz, DMSO-d₆) 1.03-1.07 (6H, m), 1.26 (3H, d), 2.26-2.34 (2H, m), 2.91 (2H, t), 3.23-3.41 (4H, m), 3.53-3.59 (1H, m), 3.69-3.73 (1H, m), 3.81 (1H, d), 3.91-3.97 (2H, m), 4.01-4.05 (1H, m), 4.29 (1H, d), 4.60 (1H, d), 6.95 (1H, s), 7.21 (1H, t), 7.28 (1H, t), 7.47 (1H, t), 7.56 (1H, d), 8.11-8.14 (1H, m), 11.27 (1H, s); m/z: (ESI+) MH⁺, 485.20.

The (R)-4-(2-chloro-6-(4-(isopropylsulfonyl)tetrahydro-2H-pyran-4-yl)pyrimidin-4-yl)-3-methylmorpholine, used as starting material, was prepared as follows: A 50% aqueous solution of NaOH (2 mL) was added to (R)-4-(2-chloro-6-(isopropylsulfonylmethyl)pyrimidin-4-yl)-3-methylmorpholine (550 mg, 1.65 mmol), tetrabutylammonium bromide (53.1 mg, 0.16 mmol) and 1-bromo-2-(2-bromoethoxy)ethane (955 mg, 4.12 mmol) in toluene (6 mL) at 22° C. The resulting mixture was stirred at 20° C. overnight. DCM (50 mL) was added and the mixture dried over sodium sulfate, filtered and then evaporated. The residue was purified by chromatography on silica eluting with a gradient of 0 to 40% EtOAc in isohexane. Pure fractions were combined and evaporated to afford (R)-4-(2-chloro-6-(4-(isopropylsulfonyl)tetrahydro-2H-pyran-4-yl)pyrimidin-4-yl)-3-methylmorpholine (450 mg, 68%); m/z: (ESI+) MH⁺, 404.16.

### EXAMPLE 3.25

### 4-(4-{4-[(1-Methylethyl)sulfonyl]piperidin-4-yl}-6-[(3R)-3-methylmorpholin-4-yl]pyrimidin-2-yl)-1H-indole

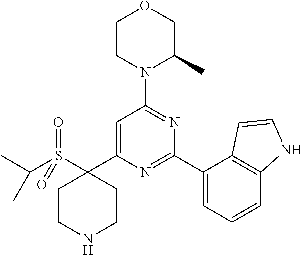

Dichlorobis(triphenylphosphine)palladium(II) (3.54 mg, 5.05 μmol) was added in one portion to (R)-tert-butyl 4-(2-chloro-6-(3-methylmorpholino)pyrimidin-4-yl)-4-(isopropylsulfonyl)piperidine-1-carboxylate (254 mg, 0.50 mmol), 2M aqueous sodium carbonate solution (0.303 mL, 0.61 mmol) and 1H-indol-4-ylboronic acid (98 mg, 0.61 mmol) in DME:water 4:1 (5 mL) at RT. The mixture was heated to 90° C. for 1 hour and then evaporated. The residue was treated with 10% TFA in DCM (5 mL) and stirred at RT for 1 hour. The mixture was purified by ion exchange chromatography, using an SCX column. The desired product was eluted from the column using 7M NH3/MeOH and fractions containing product were combined and evaporated. The residue was purified by preparative HPLC using decreasingly polar mixtures of water (containing 1% NH3) and MeCN as eluents. Fractions containing the desired compound were combined and evaporated to afford the title compound (42.0 mg, 17%); ¹H NMR (400 MHz, DMSO-d₆) 1.03 (6H, t), 1.26 (3H, d), 2.08-2.16 (2H, m), 2.46-2.50 (2H, m), 2.86 (2H, t), 2.96 (2H, t), 3.20-3.22 (1H, m), 3.39-3.41 (1H, m), 3.53-3.56 (1H, m), 3.69-3.73 (1H, m), 3.81 (1H, d), 4.01-4.04 (1H, m), 4.27 (1H, d), 4.58 (1H, d), 6.89 (1H, s), 7.21 (1H, t), 7.30 (1H, t), 7.47 (1H, t), 7.55 (1H, d), 8.12-8.14 (1H, m), 11.26 (1H, s); m/z: (ESI+) MH⁺, 484.22.

The (R)-tert-butyl 4-(2-chloro-6-(3-methylmorpholino)pyrimidin-4-yl)-4-(isopropylsulfonyl)piperidine-1-carboxylate, used as starting material, was prepared as follows:

a) A solution of (R)-4-(2-chloro-6-(isopropylsulfonylmethyl)pyrimidin-4-yl)-3-methylmorpholine (0.800 g, 2.40 mmol) in NMP (9.5 mL) was treated with sodium hydride, 60% dispersion in mineral oil (0.316 g, 7.91 mmol). The mixture was stirred at RT for 10 minutes before being treated with tetrabutylammonium bromide (1.159 g, 3.59 mmol) and N-benzyl-2-chloro-N-(2-chloroethyl)ethanamine hydrochloride (0.708 g, 2.64 mmol). The mixture was stirred for 5 minutes at RT and then heated to 50° C. for 1 hour and then at 80° C. for 1.5 hours. The mixture was allowed to cool to RT and then quenched by the addition of saturated ammonium chloride solution. The mixture was extracted with EtOAc (100 mL) and the organic solution washed with water (3×60 mL), saturated brine, dried over MgSO4, filtered, and then concentrated under reduced pressure. The residue was purified by chromatography on silica eluting with a gradient of 10 to 70% EtOAc in DCM. Pure fractions were combined and evaporated to afford (R)-4-(6-(1-benzyl-4-(isopropylsulfonyl)piperidin-4-yl)-2-chloropyrimidin-4-yl)-3-methylmorpholine (0.600 g, 51%); m/z: (ESI+) MH⁺, 493.

b) 1-Chloroethyl chloroformate (0.272 mL, 2.52 mmol) was added to a solution of (R)-4-(6-(1-benzyl-4-(isopropylsulfonyl)piperidin-4-yl)-2-chloropyrimidin-4-yl)-3-methylmorpholine (600 mg, 1.22 mmol) in DCM (10 mL). The solution was heated at reflux for 3 hours and then allowed to cool to RT. The mixture was diluted with MeOH (10 mL) and then allowed to stand overnight. The mixture was treated with di-tert-butyl dicarbonate (0.615 mL, 2.68 mmol) and N-ethyldiisopropylamine (0.421 mL, 2.43 mmol) and this solution was stirred at RT for 1.5 hours. The solution was partitioned between DCM and water and the organic phase separated and then evaporated. The residue was purified by chromatography on silica using a gradient elution of 100% DCM to 30% EtOAc/DCM. Fractions containing the desired product were combined and evaporated to afford (R)-tert-butyl 4-(2-chloro-6-(3-methylmorpholino)pyrimidin-4-yl)-4-(isopropylsulfonyl)piperidine-1-carboxylate (608 mg, 99%).

### EXAMPLE 4.01

### 4-{4-[(3S,5R)-3,5-Dimethylmorpholin-4-yl]-6-[1-(methylsulfonyl)cyclopropyl]pyrimidin-2-yl}-1H-indole (and can also be named as: 4-[4-[(3R,5S)-3,5-dimethylmorpholin-4-yl]-6-(1-methylsulfonylcyclopropyl)pyrimidin-2-yl]-1H-indole)

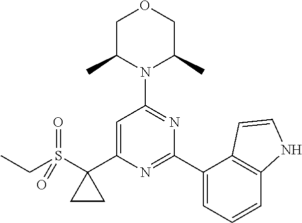

(3S,5R)-3,5-dimethyl-4-(6-(1-(methylsulfonyl)cyclopropyl)-2-(methylthio)pyrimidin-4-yl)morpholine (176 mg, 0.49 mmol) was dissolved in a mixture of dioxane (5 mL) and DMA (1 mL) and the mixture was degassed by bubbling nitrogen through it for 5 minutes. 1H-indol-4-ylboronic acid (174 mg, 1.08 mmol), (thiophene-2-carbonyloxy)copper (244 mg, 1.28 mmol) and tetrakis (triphenylphosphine)Pd(0) (45.5 mg, 0.04 mmol) were added and the resulting mixture was stirred under nitrogen at 80° C. for 16 hours. The mixture was allowed to cool to RT and purified by ion exchange chromatography, using an SCX column. The desired product was eluted from the column using 7M NH₃/MeOH; product fractions were combined and evaporated. The residue was purified by preparative HPLC using decreasingly polar mixtures of water (containing 1% NH3) and MeCN as eluents. Fractions containing the desired compound were combined and evaporated to afford the title compound (23 mg, 11%); ¹H NMR (400 MHz, DMSO) 1.26 (6H, d), 1.53 (2H, dd), 1.63 (2H, dd), 3.17 (3H, s), 3.57 (2H, dd), 3.76 (2H, d), 4.37 (2H, s), 6.70 (1H, s), 7.11 (1H, t), 7.23 (1H, d), 7.32-7.39 (1H, m), 7.45 (1H, d), 7.98 (1H, dd), 11.14 (1H, s); m/z: (ESI+) MH⁺, 427.60.

The (3S,5R)-3,5-dimethyl-4-(6-(1-(methylsulfonyl)cyclopropyl)-2-(methylthio)pyrimidin-4-yl)morpholine, used as starting material, was prepared as follows:

a) DBU (3.57 mL, 25.97 mmol) and 1,1,1-trifluoro-N-phenyl-N-(trifluoromethylsulfonyl)methanesulfonamide (9.28 g, 25.97 mmol) were added to 6-(methylsulfonylmethyl)-2-(methylthio)pyrimidin-4-ol (5.07 g, 21.64 mmol) in DCM (80 mL). The resulting mixture was stirred at RT for 72 hours. The mixture was washed with water (100 mL) and the organic phase separated and then purified by chromatography on silica eluting with DCM. Pure fractions were combined and evaporated to afford 6-(methylsulfonylmethyl)-2-(methylthio)pyrimidin-4-yl trifluoromethanesulfonate (1.98 g, 25%); ¹H NMR (400 MHz, DMSO-d₆) 2.58 (3H, s), 3.16 (3H, s), 4.80 (2H, s), 7.47 (1H, s); m/z: (ESI+) MH⁺, 366.93.

b) 6-(Methylsulfonylmethyl)-2-(methylthio)pyrimidin-4-yl trifluoromethanesulfonate (1.9 g, 5.19 mmol), (3S,5R)-3,5-dimethylmorpholine hydrochloride (1.180 g, 7.78 mmol) and N,N-diisopropylethylamine (3.61 mL, 20.74 mmol) were suspended in dioxane (20 mL) and sealed into a microwave tube. The mixture was heated to 100° C. for 2 hours and then concentrated under reduced pressure. The residue was dissolved in DCM and purified by chromatography on silica eluting with a gradient of 20 to 40% EtOAc in isohexane. Fractions containing product wre combined and evaporated to afford (3S,5R)-3,5-dimethyl-4-(6-(methylsulfonylmethyl)-2-(methylthio)pyrimidin-4-yl)morpholine (0.579 g, 34%); ¹H NMR (400 MHz, DMSO-d⁶) 1.26 (6H, d), 2.45 (3H, d), 3.11 (3H, s), 3.60 (2H, dd), 3.78 (2H, m), 4.22 (2H, s), 4.33-4.43 (2H, m), 6.58 (1H, s); m/z: (ESI+) MH⁺, 332.14.

c) A 50% aqueous solution of NaOH (1.3 mL) was added to (3S,5R)-3,5-dimethyl-4-(6-(methylsulfonylmethyl)-2-(methylthio)pyrimidin-4-yl)morpholine (541 mg, 1.63 mmol), 1,2-dibromoethane (0.141 mL, 1.63 mmol) and tetrabutylammonium bromide (52.6 mg, 0.16 mmol) in toluene (4 mL). The resulting slurry was stirred at 40° C. for 2 hours and then heated to 60° C. for 1 hour. A further portion of 1,2-dibromoethane (0.070 mL) was added and the reaction mixture was heated at 60° C. for 2 hours. A further portion of 1,2-dibromoethane (0.070 mL) was added and the reaction mixture was heated at 60° C. for 2 hours. EtOAc (20 mL) was added and the mixture washed with water (20 mL) and brine (20 mL). The organic phase was dried over MgSO₄ and then concentrated in vacuo. The residue was purified by chromatography on silica eluting with a gradient of 10 to 50% EtOAc in isohexane. Pure fractions were combined and evaporated to afford (3S,5R)-3,5-dimethyl-4-(6-(1-(methylsulfonyl)cyclopropyl)-2-(methylthio)pyrimidin-4-yl)morpholine (350 mg, 60%); m/z: (ESI+) MH⁺, 358.13.

The 6-(methylsulfonylmethyl)-2-(methylthio)pyrimidin-4-ol, used as starting material, can be prepared as described in the literature (Pike, Kurt Gordon; Finlay, Maurice Raymond Verschoyle; Fillery, Shaun Michael; Dishington, Allan Paul. Preparation of morpholinopyrimidine derivatives for treatment of proliferative disease. PCT Int. Appl. (2007), WO2007080382).

The (3S,5R)-3,5-dimethylmorpholine, used as starting material, can be prepared as described in the literature (Morris, Jeffrey James; Pike, Kurt Gordon. Pyrimidine derivatives that are useful in the treatment of diseases mediated by mTOR and/or PI3K enzyme and their preparation. PCT Int. Appl. (2009), WO2009007748)

### EXAMPLE 5.01

### 4-{4-[1-(Methylsulfonyl)cyclopropyl]-6-(8-oxa-3-azabicyclo[3.2.1]oct-3-yl)pyrimidin-2-yl}-1H-indole

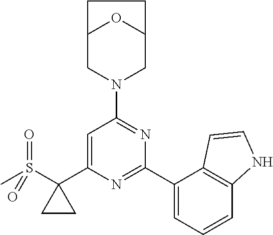

1H-Indol-4-ylboronic acid (149 mg, 0.93 mmol), 3-(6-(1-(methylsulfonyl)cyclopropyl)-2-(methylthio)pyrimidin-4-yl)-8-oxa-3-azabicyclo[3.2.1]octane (150 mg, 0.42 mmol), tetrakis(triphenylphosphine)palladium(0) (39.0 mg, 0.03 mmol) and (thiophene-2-carbonyloxy)copper (209 mg, 1.10 mmol) were suspended in dioxane (5 mL) and the mixture degassed with nitrogen. The mixture was heated to 80° C. for 18 hours (with vigourous stiffing). The mixture was cooled and purified by ion exchange chromatography, using an SCX column. The desired product was eluted from the column using 0.35M NH3/MeOH; pure fractions were combined and evaporated. The residue was purified by preparative HPLC using decreasingly polar mixtures of water (containing 1% NH3) and MeOH/MeCN (3/1) as eluents. Fractions containing product were combined and evaporated to afford the title compound (65 mg, 36%); ¹H NMR (400 MHz, DMSO-d₆) 1.60-1.63 (dd, 2H), 1.70-1.78 (m, 4H), 1.85-1.91 (m, 2H), 3.17-3.20 (m, 2H), 3.31 (s, 3H), 4.05-4.25 (m, 2H), 4.49-4.54 (m, 2H), 6.81 (s, 1H), 7.20 (t, 1H), 7.30 (t, 1H), 7.46 (t, 1H), 7.55 (d, 1H), 8.04 (d, 1H), 11.25 (s, 1H); m/z: (ESI+) MH⁺, 425.11.

The 3-(6-(1-(methylsulfonyl)cyclopropyl)-2-(methylthio)pyrimidin-4-yl)-8-oxa-3-azabicyclo[3.2.1]octane, used as starting material, was prepared as follows:

a) 8-oxa-3-azabicyclo[3.2.1]octane (627 mg, 5.54 mmol) was added to 4-chloro-6-(methylsulfonylmethyl)-2-(methylthio)pyrimidine (700 mg, 2.77 mmol) and DIPEA (1.447 mL, 8.31 mmol) in DCM (10 mL). The resulting mixture was stirred at rt for 3 days. The reaction mixture was washed sequentially with 1M HCl (2×10 mL), water (10 mL) and saturated brine (10 mL). The organic layer was dried over MgSO4, filtered and then evaporated. The residue was purified by chromatography on silica eluting with a gradient of 0 to 10% MeOH in DCM. Pure fractions were combined and evaporated to afford 346-(methylsulfonylmethyl)-2-(methylthio)pyrimidin-4-yl)-8-oxa-3-azabicyclo[3.2.1]octane (400 mg, 44%); m/z: (ESI+) MH⁺, 330.06.

b) Sodium hydroxide (50% aqueous solution) (1.781 mL, 66.78 mmol) was added to 346-(methylsulfonylmethyl)-2-(methylthio)pyrimidin-4-yl)-8-oxa-3-azabicyclo[3.2.1]octane (400 mg, 1.21 mmol), 1,2-dibromoethane (0.314 mL, 3.64 mmol) and tetrabutylammonium bromide (39.1 mg, 0.12 mmol) in toluene (20 mL). The resulting mixture was stirred at 60° C. for 2 hours. Water (50 mL) was added and the mixture was extracted with toluene (20 mL×2). The toluene layers were dried over MgSO4, filtered and evaporated. The residue was purified by chromatography on silica eluting with a gradient of 0 to 10% MeOH in DCM. Pure fractions were combined and evaporated to afford 3-(6-(1-(methylsulfonyl)cyclopropyl)-2-(methylthio)pyrimidin-4-yl)-8-oxa-3-azabicyclo[3.2.1]octane (351 mg, 81%); m/z: (ESI+) MH⁺, 356.09.

The 4-chloro-6-(methylsulfonylmethyl)-2-(methylthio)pyrimidine, used as starting material, can be prepared as described in the literature (Finlay, Maurice Raymond Verschoyle. Morpholinopyrimidine derivatives, processes for preparing them, pharmaceutical compositions containing them, and their use for treating proliferative disorders. PCT Int. Appl. (2008), WO2008023180).

The 8-oxa-3-azabicyclo[3.2.1]octane, used as starting material, may be prepared as described in the literature (Feurer, Achim; Luithle, Joachim; Wirtz, Stephan-nicholas; Koenig, Gerhard; Stasch, Johannes-peter; Stahl, Elke; Schreiber, Rudy; Wunder, Frank; Lang, Dieter. Preparation of Pyrazolopyridinylpyrimidines as Inhibitors of Cgmp Degradation for the treatment of central nervous system diseases. PCT Int. Appl. (2004), WO2004009589).

1. A compound of formula (I): 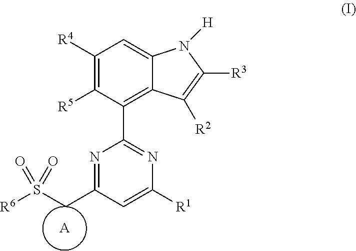
wherein:
Ring A is a C₃₋₆cycloalkyl or a saturated 4-6 membered heterocyclic ring containing one heteroatom selected from O, N and S;
R¹ is selected from morpholin-4-yl, 3-methylmorpholin-4-yl, 3,5-dimethylmorpholin-4-yl and a 8-oxa-3-azabicyclo[3.2.1]octan-3-yl group;
R² and R⁵ are hydrogen;
R³ is hydrogen or methy;
R⁴ is selected from hydrogen, methyl, fluoro, chloro, cyano and methoxy; and
R⁶ is a group selected from methyl, ethyl, i-propyl and cyclopropyl;
or a pharmaceutically acceptable salt thereof.

2. A compound according to claim 1 wherein Ring A is a unsubstituted C₃₋₆cycloalkyl or a saturated 4-6 heterocyclic ring containing one heteroatom selected from O and N.

3. A compound according to claim 1 wherein Ring A is a cyclopropyl, tetrahydropyranyl or piperidinyl ring.

4. A compound according to claim 1 wherein
either
R³ is hydrogen; and R⁴ is selected from hydrogen, methyl, fluoro, chloro, cyano and methoxy;
or
R⁴ is hydrogen, and R³ is hydrogen or methyl.

5. A compound according to claim 1 wherein R³ is hydrogen and R⁴ is hydrogen.

6. A compound according to claim 1 wherein R¹ is 3-methylmorpholin-4-yl.

7. A compound according to claim 1 wherein R⁶ is a group selected from methyl, ethyl, i-propyl and cyclopropyl.

8. A compound according to claim 1 wherein R⁶ is methyl.

9. A compound according to claim 1 where the compound of formula (I) is a compound of formula (Ia), 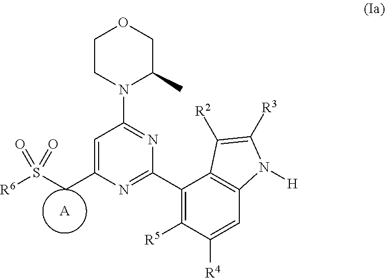
or a pharmaceutically acceptable salt thereof.

10. A compound according to claim 9, or a pharmaceutically acceptable salt, thereof wherein:
Ring A is a cyclopropyl, tetrahydropyranyl or piperidinyl ring;
R² is hydrogen;
R³ is hydrogen;
R⁴ is hydrogen;
R⁵ is hydrogen; and
R⁶ is a methyl group.

11. A compound according to claim 1 wherein the compound of formula (I) is selected from any one of
4-{4-[1-(Methylsulfonyl)cyclopropyl]-6-morpholin-4-ylpyrimidin-2-yl}-1H-indole;
6-Methyl-4-{4-[1-(methylsulfonyl)cyclopropyl]-6-morpholin-4-ylpyrimidin-2-yl}-1H-indole;
2-Methyl-4-{4-[1-(methylsulfonyl)cyclopropyl]-6-morpholin-4-ylpyrimidin-2-yl}-1H-indole;
6-Methoxy-4-{4-[1-(methylsulfonyl)cyclopropyl]-6-morpholin-4-ylpyrimidin-2-yl}-1H-indole;
4-{4-[1-(methylsulfonyl)cyclopropyl]-6-morpholin-4-ylpyrimidin-2-yl}-1H-indole-6-carbonitrile;
6-chloro-4-{4-[1-(methylsulfonyl)cyclopropyl]-6-morpholin-4-ylpyrimidin-2-yl}-1H-indole;
6-fluoro-4-{4-[1-(methylsulfonyl)cyclopropyl]-6-morpholin-4-ylpyrimidin-2-yl}-1H-indole;
4-{4-[4-(Methylsulfonyl)piperidin-4-yl]-6-morpholin-4-ylpyrimidin-2-yl}-1H-indole;
4-{4-[4-(Methylsulfonyl)tetrahydro-2H-pyran-4-yl]-6-morpholin-4-ylpyrimidin-2-yl}-1H-indole;
4-{4-[1-(ethylsulfonyl)cyclopropyl]-6-morpholin-4-ylpyrimidin-2-yl}-1H-indole;
4-(4-{1-[(1-methylethyl)sulfonyl]cyclopropyl}-6-morpholin-4-ylpyrimidin-2-yl)-1H-indole;
4-{4-[1-(cyclopropylsulfonyl)cyclopropyl]-6-morpholin-4-ylpyrimidin-2-yl}-1H-indole;
4-{4-[4-(cyclopropylsulfonyl)tetrahydro-2H-pyran-4-yl]-6-morpholin-4-ylpyrimidin-2-yl}-1H-indole;
4-{4-[4-(cyclopropylsulfonyl)piperidin-4-yl]-6-morpholin-4-ylpyrimidin-2-yl}-1H-indole;
4-{4-[4-(Cyclopropylsulfonyl)piperidin-4-yl]-6-[(3S)-3-methylmorpholin-4-yl]pyrimidin-2-yl}-1H-indole;
4-{4-[4-(Cyclopropylsulfonyl)tetrahydro-2H-pyran-4-yl]-6-[(3S)-3-methylmorpholin-4-yl]pyrimidin-2-yl}-1H-indole;
4-{4-[(3S)-3-Methylmorpholin-4-yl]-6-[1-(methylsulfonyl)cyclopentyl]pyrimidin-2-yl}-1H-indole;
4-{4-[(3S)-3-Methylmorpholin-4-yl]-6-[4-(methylsulfonyl)tetrahydro-2H-pyran-4-yl]pyrimidin-2-yl}-1H-indole;
4-{4-[(3S)-3-Methylmorpholin-4-yl]-6-[1-(methylsulfonyl)-cyclopropyl]pyrimidin-2-yl)-1H-indole;
4-{4-[(3R)-3-Methylmorpholin-4-yl]-6-[1-(methylsulfonyl)cyclopropyl]pyrimidin-2-yl}-1H-indole;
6-Methyl-4-{4-[(3R)-3-methylmorpholin-4-yl]-6-[1-(methylsulfonyl)cyclopropyl]pyrimidin-2-yl}-1H-indole;
2-Methyl-4-{4-[(3R)-3-methylmorpholin-4-yl]-6-[1-(methylsulfonyl)cyclopropyl]pyrimidin-2-yl}-1H-indole;
4-{4-[(3R)-3-Methylmorpholin-4-yl]-6-[1-(methylsulfonyl)cyclopropyl]pyrimidin-2-yl}-1H-indole-6-carbonitrile;
6-chloro-4-{4-[(3R)-3-methylmorpholin-4-yl]-6-[1-(methylsulfonyl)cyclopropyl]pyrimidin-2-yl}-1H-indole;
6-fluoro-4-{4-[(3R)-3-methylmorpholin-4-yl]-6-[1-(methylsulfonyl)cyclopropyl]pyrimidin-2-yl}-1H-indole;
6-methoxy-4-{4-[(3R)-3-methylmorpholin-4-yl]-6-[1-(methylsulfonyl)cyclopropyl]pyrimidin-2-yl}-1H-indole;
4-{4-[(3R)-3-Methylmorpholin-4-yl]-6-[4-(methylsulfonyl)tetrahydro-2H-pyran-4-yl]pyrimidin-2-yl}-1H-indole;
6-Methyl-4-{4-[(3R)-3-methylmorpholin-4-yl]-6-[4-(methylsulfonyl)tetrahydro-2H-pyran-4-yl]pyrimidin-2-yl}-1H-indole;
2-Methyl-4-{4-[(3R)-3-methylmorpholin-4-yl]-6-[4-(methylsulfonyl)tetrahydro-2H-pyran-4-yl]pyrimidin-2-yl}-1H-indole;
6-Methoxy-4-{4-[(3R)-3-methylmorpholin-4-yl]-6-[4-(methylsulfonyl)tetrahydro-2H-pyran-4-yl]pyrimidin-2-yl}-1H-indole;
6-chloro-4-{4-[(3R)-3-methylmorpholin-4-yl]-6-[4-(methylsulfonyl)tetrahydro-2H-pyran-4-yl]pyrimidin-2-yl}-1H-indole;
6-fluoro-4-{4-[(3R)-3-methylmorpholin-4-yl]-6-[4-(methylsulfonyl)tetrahydro-2H-pyran-4-yl]pyrimidin-2-yl}-1H-indole;
4-{4-[(3R)-3-methylmorpholin-4-yl]-6-[4-(methylsulfonyl)tetrahydro-2H-pyran-4-yl]pyrimidin-2-yl}-1H-indole-6-carbonitrile;
4-{4-[(3R)-3-Methylmorpholin-4-yl]-6-[4-(methylsulfonyl)piperidin-4-yl]pyrimidin-2-yl}-1H-indole;
4-{4-[(3R)-3-Methylmorpholin-4-yl]-6-[1-(methylsulfonyl)cyclobutyl]pyrimidin-2-yl}-1H-indole;
4-{4-[1-(Cyclopropylsulfonyl)cyclopropyl]-6-[(3R)-3-methylmorpholin-4-yl]pyrimidin-2-yl}-1H-indole;
4-{4-[4-(Cyclopropylsulfonyl)piperidin-4-yl]-6-[(3R)-3-methylmorpholin-4-yl]pyrimidin-2-yl}-1H-indole;
4-{4-[4-(Cyclopropylsulfonyl)tetrahydro-2H-pyran-4-yl]-6-[(3R)-3-methylmorpholin-4-yl]pyrimidin-2-yl}-1H-indole;
4-{4-[1-(Ethylsulfonyl)cyclopropyl]-6-[(3R)-3-methylmorpholin-4-yl]pyrimidin-2-yl}-1H-indole;
4-{4-[4-(Ethylsulfonyl)tetrahydro-2H-pyran-4-yl]-6-[(3R)-3-methylmorpholin-4-yl]pyrimidin-2-yl}-1H-indole;
4-{4-[4-(Ethylsulfonyl)piperidin-4-yl]-6-[(3R)-3-methylmorpholin-4-yl]pyrimidin-2-yl}-1H-indole;
4-(4-{1-[(1-Methylethyl)sulfonyl]cyclopropyl}-6-[(3R)-3-methylmorpholin-4-yl]pyrimidin-2-yl)-1H-indole;
4-(4-{4-[(1-Methylethyl)sulfonyl]tetrahydro-2H-pyran-4-yl}-6-[(3R)-3-methylmorpholin-4-yl]pyrimidin-2-yl)-1H-indole;
4-(4-{4-[(1-Methylethyl)sulfonyl]piperidin-4-yl}-6-[(3R)-3-methylmorpholin-4-yl]pyrimidin-2-yl)-1H-indole;
4-{4-[(3S,5R)-3,5-Dimethylmorpholin-4-yl]-6-[1-(methylsulfonyl)cyclopropyl]pyrimidin-2-yl}-1H-indole;
4-[4-[(3R,5S)-3,5-dimethylmorpholin-4-yl]-6-(1-methylsulfonylcyclopropyl)pyrimidin-2-yl]-1H-indole); and
4-{4-[1-(Methylsulfonyl)cyclopropyl]-6-(8-oxa-3-azabicyclo[3.2.1]oct-3-yl)pyrimidin-2-yl}-1H-indole, or a pharmaceutically acceptable salt thereof.

12. A pharmaceutical composition comprising a compound of formula (I), or a pharmaceutically acceptable salt thereof, according to claim 1 in association with a pharmaceutically acceptable adjuvant, diluent or carrier.

13. A method for treating cancer which comprises administering to a patient in need thereof a therapeutically effective amount of a compound of formula (I), or a pharmaceutically acceptable salt thereof, according to claim 1.

14. A method for treating cancer which comprises administering to a patient in need thereof a therapeutically effective amount of a compound of formula (Ia), or a pharmaceutically acceptable salt thereof, according to claim 9.

15. A method for treating cancer which comprises administering to a patient in need thereof a therapeutically effective amount of a compound of formula (I), or a pharmaceutically acceptable salt thereof, according to claim 11.
